(19) United States
(12) Patent Application Publication
(10) Pub. No.: US 2023/0278027 A1
(43) Pub. Date: Sep. 7, 2023

(54) METHODS AND APPARATUSES FOR DETECTING BIOMOLECULES

(71) Applicant: Randall Davis, Pleasanton, CA (US)

(72) Inventors: Randall DAVIS, Pleasanton, CA (US); Alison ORSI-DAVIS, Pleasanton, CA (US)

(21) Appl. No.: 18/112,665

(22) Filed: Feb. 22, 2023

Related U.S. Application Data
(63) Continuation of application No. PCT/US21/46869, filed on Aug. 20, 2021.
(60) Provisional application No. 63/069,530, filed on Aug. 24, 2020.

(51) Int. Cl. B01L 3/00; C12Q 1/6844; G01N 33/543

(52) U.S. Cl. CPC B01L 3/502707; B01L 3/502715; B01L 3/527; C12Q 1/6844; G01N 33/5438; B01L 2200/16; B01L 2300/0645; B01L 2300/126

(57) ABSTRACT

The present disclosure provides methods and systems for detecting a presence or absence of analytes. The systems may comprise membrane-based substrates.

### CROSS-REFERENCE

This application is a continuation of International Application No. PCT/US2021/046869, filed Aug. 20, 2021, which claims the benefit of U.S. Provisional Patent Application No. 63/069,530, filed Aug. 24, 2020, both of which are incorporated herein by reference in their entirety.

### BACKGROUND

Global health crises, such as the recent COVID-19 pandemic, demonstrate the importance of rapid, reliable, and accessible diagnostic tests. Testing that is inexpensive and able to be performed by an untrained user can permit daily screening or winnowing to help catch potentially infected individuals before they are able to go out into the general population and possibly spread a disease.

### SUMMARY

Recognized herein is a need for low cost, personally administrable, rapid testing for a variety of diseases or conditions. Additionally recognized herein is a need for a disposable system for cell lysis and concentration, sample amplification, and sample detection that can be performed in a home setting.

In an aspect, the present disclosure provides a device for assaying a presence or an absence of an analyte, comprising: a substrate comprising at least two composite electrodes configured to capture the analyte and detect a signal indicative of the presence or absence of the analyte, upon or subsequent to contact of the analyte with the substrate.

In another aspect, the present disclosure provides a device, comprising: a three-dimensional membrane-based substrate comprising at least a first location and a second location, wherein the first location comprises a first substance specific for a first analyte and configured to facilitate generation of a first signal indicative of a presence of the first analyte, upon or subsequent to contact of the first substance with the first analyte, and wherein the second location comprises a second substance specific for a second analyte different from the first analyte and configured to facilitate generation of a second signal indicative of a presence of the second analyte, upon or subsequent to contact of the second substance with the second analyte.

In some embodiments, the device further comprises a detection unit in sensory communication with the first location or the second location. In some embodiments, the detection unit is configured to detect a presence or absence of the first signal or the second signal from the first location or the second location, thereby determining a presence or absence of the first analyte or the second analyte. In some embodiments, the detection unit is a personal device of a subject. In some embodiments, the subject is a person suspected of having a condition or disease, and wherein the presence or absence of the first analyte or the second analyte is indicative of the subject having the condition or disease. In some embodiments, the subject is a health care provider. In some embodiments, the detection unit is a smartphone. In some embodiments, the substrate comprises a plurality of locations each comprising a substance specific for a different analyte. In some embodiments, the substrate comprises a structure. In some embodiments, the structure is configured to receive and retain a sample suspected of having the first analyte or the second analyte. In some embodiments, the structure comprises the first location and the second location. In some embodiments, the substrate comprises a plurality of structures. In some embodiments, the plurality of the structures comprises at least a first structure corresponding to the first location and a second structure corresponding to the second location. In some embodiments, the first location and the second location are spatially separated. In some embodiments, the first location and the second location overlap. In some embodiments, the structure is a microstructure. In some embodiments, the structure is a well. In some embodiments, the structure is a fluidic channel. In some embodiments, the fluidic channel is configured to facilitate detection of a different analyte. In some embodiments, the structure comprises a plurality of locations each comprising a substance specific for a different analyte. In some embodiments, individual locations of the plurality of locations are individually addressable, and optionally wherein the plurality of locations are asynchronously addressable. In some embodiments, individual locations of the plurality of locations are individually addressable and individually controllable. In some embodiments, individual locations of the plurality of locations are addressed and controlled asynchronously. In some embodiments, the plurality of locations comprises at least about 10 locations. In some embodiments, the first analyte or the second analyte comprises nucleic acid molecules. In some embodiments, the first substance or the second substance comprises a primer. In some embodiments, the membrane-based substrate comprises one or more additional locations. In some embodiments, the one or more additional locations are configured to act as a positive or negative control. In some embodiments, the device further comprises one or more additional membrane-based substrates. In some embodiments, the device is a portable device. In some embodiments, the device further comprises one or more bottles configured to contain one or more reagents. In some embodiments, the one or more bottles comprise a feedback module. In some embodiments, the feedback module is configured to provide information regarding the application of the one or more reagents from the one or more bottles. In some embodiments, the feedback module comprises an electronic feedback module. In some embodiments, the electronic feedback module comprises a conductive module, a capacitive module, a resistive module, or any combination thereof.

In another aspect, the present disclosure provides a method, comprising: (a) directing a sample suspected of having a first analyte or a second analyte different from the first analyte to a device, the device comprising: a three-dimensional membrane-based substrate, wherein the membrane-based substrate comprises at least a first location and a second location, wherein the first location comprises a first substance specific for the first analyte and configured to facilitate generation of a first signal indicative of a presence of the first analyte, upon or subsequent to contact of the first substance with the first analyte, and wherein the second location comprises a second substance specific for the second analyte and configured to facilitate generation of a second signal indicative of a presence of the second analyte, upon or subsequent to contact of the second substance with the second analyte; (b) detecting a presence or absence of the first signal or the second signal from the first location or the second location, upon or subsequent to the sample being directed to the device; and (c) determining a presence or absence of the first analyte or the second analyte, based on the presence or absence of the first signal or the second signal detected in (b).

In some embodiments, the first signal or the second signal comprises a signal increase relative to a baseline. In some embodiments, the first signal or the second signal comprises a signal decrease relative to a baseline. In some embodiments, the sample is from a subject suspected of having a disease or condition. In some embodiments, the sample is from a subject suspected of being infected with a pathogen. In some embodiments, the pathogen is severe acute respiratory syndrome coronavirus-19 (SARS-CoV-2) In some embodiments, the first analyte or the second analyte comprises nucleic acid molecules In some embodiments, the first substance or the second substance comprises a primer In some embodiments, the nucleic acid molecules comprise a ribonucleic acid (RNA) of SARS-CoV-2 or a fragment thereof. In some embodiments, the first signal or the second signal is a colorimetric signal. In some embodiments, the detecting the first signal or the second signal comprises detecting a color calibration panel disposed adjacent to the first location or the second location. In some embodiments, the first signal or the second signal is an electrical signal. In some embodiments, the electrical signal is related to or generated by a change in a pH of a solution comprising the first or second analyte. In some embodiments, the electrical signal may be detected by a change in a conductivity across an electrode resulting from an oxidation or reduction reaction. In some embodiments, one of the first signal or the second signal is a colorimetric signal and the other of the first signal or the second signal is an electrical signal. In some embodiments, the sample has a volume of at least about 500 microliters. In some embodiments, the sample does not comprise a pH buffer. In some embodiments, the sample is mixed with a reaction mixture comprising a pH buffer. In some embodiments, the sample comprises saliva, blood, or a combination thereof. In some embodiments, the sample is taken from a subject using a swab. In some embodiments, the swab comprises a breakable head. In some embodiments, the method further comprises outputting a report that identifies the presence or absence of the first analyte or the second analyte. In some embodiments, the report comprises one or more color codes indicative of the first analyte or the second analyte. In some embodiments, the method further comprises displaying the report on a personal device of a subject. In some embodiments, the subject is a subject from which the sample is obtained. In some embodiments, the subject is a health care provider. In some embodiments, the personal device is a mobile device. In some embodiments, the mobile device comprises a light, and wherein the light is configured to illuminate a colorimetric signal. In some embodiments, the personal device is in communication with the device. In some embodiments, the detecting the presence or absence of the first signal or the second signal occurs at a temperature of at least about 35° C. In some embodiments, the first analyte or the second analyte comprises nucleic acid molecules. In some embodiments, the first substance or the second substance comprises a primer.

In another aspect, the present disclosure provides a device, comprising: two or more fluidic chambers; a fluidic channel between and in fluidic communication with the two or more fluidic chambers; and a valve disposed adjacent to or within the fluidic channel, the valve (i) comprising a chamber that is compressible or expandable, and (ii) configured to regulate fluid flow between the two or more fluidic chambers upon an actuation of the chamber.

In some embodiments, the chamber is a plastic encased bubble. In some embodiments, the plastic encased bubble is a plastic encased air bubble. In some embodiments, the actuation comprises an application of a pressure to the chamber. In some embodiments, the pressure is a positive pressure. In some embodiments, the pressure is applied manually. In some embodiments, the valve comprises a pressure breakable seal. In some embodiments, a thickness of a membrane wall of the valve is less than a thickness of a wall of the fluidic channel. In some embodiments, a thickness of the membrane wall is at most about 1 millimeter. In some embodiments, the device is a single tube. In some embodiments, the device is a cartridge. In some embodiments, the cartridge comprises a plurality of valves connecting a plurality of fluidic chambers. In some embodiments, the device comprises the valve between a sample chamber and a reagent chamber. In some embodiments, the sample chamber and the reagent chamber are affixed to a rigid support. In some embodiments, the chamber is filled with pressurized gas. In some embodiments, the chamber is filled with non-pressurized gas. In some embodiments, the device is a portable device. In some embodiments, the chamber is configured to not break upon the application of the pressure on only one side of the chamber. In some embodiments, the chamber is inflated.

In another aspect, the present disclosure provides a method, comprising: (a) directing a fluid to a device comprising: two or more fluidic chambers; a fluidic channel between and in fluidic communication with the two or more fluidic chambers; and a valve disposed adjacent to or within the fluidic channel, the valve (i) comprising a chamber that is compressible or expandable, and (ii) configured to regulate fluid flow between the two or more fluidic chambers upon actuation of the chamber; and (b) actuating the chamber to regulate fluid flow between the two or more fluidic chambers.

In some embodiments, the regulating fluid flow comprises bursting the chamber. In some embodiments, the actuation comprises applying a pressure to the chamber. In some embodiments, the pressure is applied on a center of the chamber. In some embodiments, the pressure is a pressure of at most about 0.5 megapascals. In some embodiments, the method further comprises actuating the chamber a second time. In some embodiments, the actuating the chamber the second time comprises applying a pressure to fully break the chamber.

In another aspect, the present disclosure provides a device, comprising: an inlet configured to receive a sample; a fluidic channel in fluidic connection with the inlet and a fluidic region downstream of the inlet, the fluidic channel configured to passively or actively flow the sample from the inlet to the fluidic region upon receipt of the sample; and at least one electrode adjacent to and operably coupled to the fluidic region, the at least one electrode configured to (1) enrich for one or more analytes from the sample in the fluidic region, (2) subject the one or more analytes to one or more reactions under conditions sufficient to yield a signal indicative of a presence or absence of an analyte among the one or more analytes, and (3) detect the signal from the fluidic region, thereby determining the presence or absence of the analyte in the sample.

In some embodiments, the at least one electrode is configured to apply an electric field of at most about 3 V to concentrate the one or more analytes. In some embodiments, the at least one electrode is configured to subject the one or more analytes to a temperature from about 30° C. to about 75° C. In some embodiments, the temperature is from about 35° C. to about 40° C. In some embodiments, the device comprises a heating element configured to subject the one or more analytes to a temperature from about 30° C. to about 75° C. In some embodiments, the temperature is from about 35° C. to about 40° C. In some embodiments, the at least one electrode comprises two or more electrodes configured in a concentric arrangement. In some embodiments, the two or more concentric electrodes are configured to be sequentially charged to sequentially enrich for the one or more analytes. In some embodiments, the at least one electrode comprises a material selected from the group consisting of gold, silver, copper, and conductive carbon. In some embodiments, the at least one electrode comprises a conductive carbon membrane. In some embodiments, the at least one electrode comprises at least one material selected from the group consisting of silicon oxides, zinc oxide, and titanium oxide. In some embodiments, the material comprises nanoparticles. In some embodiments, the material is functionalized. In some embodiments, the material is functionalized with one or more of nucleotides, oligonucleotides, antimers, antibodies, chelators, or proteins. In some embodiments, the device is a portable device. In some embodiments, the device further comprises one or more bottles configured to contain one or more reagents. In some embodiments, the one or more bottles comprise a feedback module. In some embodiments, the feedback module is configured to provide information regarding the application of the one or more reagents from the one or more bottles. In some embodiments, the feedback module comprises an electronic feedback module. In some embodiments, the electronic feedback module comprises a conductive module, a capacitive module, a resistive module, or any combination thereof.

In another aspect, the present disclosure provides a method, comprising: (a) directing a sample to a device comprising: an inlet configured to receive the sample; a fluidic channel in fluidic connection with the inlet and a fluidic region downstream of the inlet, the fluidic channel configured to passively or actively flow the sample from the inlet to the fluidic region upon receipt of the sample; and at least one electrode adjacent to and operably coupled to the fluidic region, the at least one electrode configured to (1) enrich for one or more analytes from the sample in the fluidic region, (2) subject the one or more analytes to one or more reactions under conditions sufficient to yield a signal indicative of a presence or absence of a analyte among the one or more analytes, and (3) detect the signal from the fluidic region, thereby determining the presence or absence of the analyte in the sample; (b) passively or actively flow the sample from the inlet to the fluidic region via the fluidic channel; (c) using the at least one electrode to, in the fluidic region, enrich for one or more analytes from the sample, and subject the one or more analytes to one or more reactions under conditions sufficient to yield a signal indicative of a presence or absence of an analyte among the one or more analytes; and (d) detecting the signal from the fluidic region using the at least one electrode, thereby determining the presence or absence of the analyte in the sample.

In some embodiments, the enriching for one or more analytes comprises applying an electric field to the sample, or wherein the subjecting the one or more analytes to the reaction comprises using the at least one electrode to heat the one or more analytes. In some embodiments, the at least one electrode comprises a plurality of electrodes, and wherein individual electrodes of the plurality of electrodes are configured to, individually or collectively, perform one or more of the (1)-(3). In some embodiments, the one or more analytes comprises one or more nucleic acids, and wherein the heating is sufficient to perform an isothermal amplification reaction of the one or more nucleic acids. In some embodiments, the detecting the signal comprises detecting an electrical signal. In some embodiments, the electrical signal is generated by a change in a pH of the sample. In some embodiments, the electrical signal is generated by a change in an electrical property across an electrode due to an oxidation or reduction reaction. In some embodiments, the change in the pH is due to an amplification of one or more nucleic acids within the sample. In some embodiments, the detecting the signal comprises detecting an optical signal. In some embodiments, the optical signal is a colorimetric optical signal generated by a change in a pH of the sample. In some embodiments, the colorimetric optical signal comprises a change in color of a pH indicator. In some embodiments, the device further comprises a color standard positioned to be viewable when performing the detecting. In some embodiments, the color standard is used to calibrate for a color of the optical signal. In some embodiments, the optical signal is a colorimetric optical signal generated by an enzymatic oxidation or reduction of a substrate. In some embodiments, the enzymatic oxidation or reduction of the substrate comprises the use of a horseradish peroxidase. In some embodiments, the method further comprises pulsing an electrical current through the at least one electrode to mix the one or more analytes with one or more reagents.

In another aspect, the present disclosure provides a device, comprising: a membrane-based substrate comprising (1) a recess configured to receive and retain a sample having a volume of less than or equal to about 5 microliters (μL), and (2) a surface comprising a substance specific for an analyte and configured to facilitate generation of a signal indicative of a presence or absence of the analyte in the sample, upon or subsequent to contact of the sample with the surface.

In some embodiments, the volume is less than or equal to about 1 μL. In some embodiments, the signal is an electrical signal. In some embodiments, the electrical signal is related to or generated by a change in a pH of the sample. In some embodiments, the electrical signal may be detected by a change in a conductivity across an electrode resulting from an oxidation or reduction reaction. In some embodiments, the signal is an optical signal. In some embodiments, the optical signal is a colorimetric signal. In some embodiments, the colorimetric signal is generated by a change in color of a pH indicator. In some embodiments, the colorimetric signal is generated by an enzymatic oxidation or reduction of a substrate. In some embodiments, the enzymatic oxidation or reduction of a substrate comprises the use of a horseradish peroxidase In some embodiments, the membrane-based substrate comprises a plurality of recesses. In some embodiments, each of the plurality of recesses comprises a surface each comprising a substance specific for a different analyte and configured to facilitate generation of a signal indicative of a presence or absence of the different analytes in the sample, upon or subsequent to contact of the sample with the plurality of recesses. In some embodiments, the device further comprises at least one electrode. In some embodiments, the at least one electrode is configured to detect the signal. In some embodiments, the device further comprises a plurality of membrane-based substrates in an array. In some embodiments, each membrane-based substrate of the membrane-based substrates comprises a surface comprising substances specific for a different analyte of a plurality of analytes. In some embodiments, at least a portion of the membrane-based substrate is adjacent to or part of a conductive region.

In another aspect, the present disclosure provides a method, comprising: (a) directing a sample to a device, the device comprising a membrane-based substrate having (1) a recess configured to receive and retain less than or equal to about 5 microliters (μL) the sample and (2) a surface comprising a substance specific for a analyte and configured to facilitate generation of a signal indicative of a presence or absence of the analyte in the sample, upon or subsequent to contact of the sample with the surface; and (b) detecting the signal from the surface upon or subsequent to contact of the sample with the surface, thereby determining a presence or absence of the analyte in the sample.

In some embodiments, the sample is from a subject suspected of having a disease or condition In some embodiments, the sample is from a subject suspected of being infected with a pathogen In some embodiments, the pathogen is severe acute respiratory syndrome coronavirus-19 (SARS-CoV-2) In some embodiments, the analyte comprises nucleic acid molecules In some embodiments, the substance comprises a primer In some embodiments, the nucleic acid molecules comprise a ribonucleic acid (RNA) of SARS-CoV-2 or a fragment thereof. In some embodiments, the signal is an electrical signal. In some embodiments, the electrical signal is related to or generated by a change in a pH of the sample. In some embodiments, the electrical signal may be detected by a change in a conductivity across an electrode resulting from an oxidation or reduction reaction. In some embodiments, the signal is an optical signal. In some embodiments, the optical signal is a colorimetric signal. In some embodiments, the colorimetric signal is generated by a change in color of a pH indicator.

In another aspect, the present disclosure provides a device, comprising: a membrane-based substrate comprising a recess configured to receive and retain (i) a sample having a volume of less than or equal to about 2 milliliters and (ii) a substance specific for a analyte and configured to facilitate generation of a signal indicative of a presence or absence of the analyte in the sample, upon or subsequent to contact of the sample with the surface; and a control unit configured to subject the sample and the substance to one or more reactions under conditions sufficient to generate the signal within 60 minutes (min) subsequent to receipt of the sample.

In some embodiments, the volume is less than or equal to about 1 milliliter. In some embodiments, the signal is an electrical signal. In some embodiments, the electrical signal is related to or generated by a change in a pH of the sample. In some embodiments, the electrical signal may be detected by a change in a conductivity across an electrode resulting from an oxidation or reduction reaction. In some embodiments, the signal is an optical signal. In some embodiments, the optical signal is a colorimetric signal. In some embodiments, the colorimetric signal is generated by a change in color of a pH indicator. In some embodiments, the colorimetric signal is generated by an enzymatic oxidation or reduction of a substrate. In some embodiments, the enzymatic oxidation or reduction of a substrate comprises the use of a horseradish peroxidase. In some embodiments, the membrane-based substrate comprises a plurality of recesses. In some embodiments, each recess of the plurality of recesses comprises a surface each comprising a substance specific for a different analyte and configured to facilitate generation of a signal indicative of a presence or absence of the different analytes in the sample, upon or subsequent to contact of the sample with the plurality of recesses.

In another aspect, the present disclosure provides a method, comprising: (a) directing (i) a sample having a volume of less than or equal to about 2 milliliters and (ii) a substance to a device, the device comprising a recess configured to receive and retain the sample and the substance, which substance is specific for an analyte and configured to facilitate generation of a signal indicative of a presence or absence of the analyte in the sample, upon or subsequent to contact of the sample with the surface; (b) subjecting the sample and the substance to one or more reactions under conditions sufficient to generate the signal; and (c) detecting the signal from the substrate, thereby determining a presence or absence of the analyte in the sample, wherein (a)-(c) are separated in time by less than or equal to about 45 minutes (min).

In some embodiments, (a)-(c) are separated in time by less than or equal to 15 min. In some embodiments, (a)-(c) are separated in time by less than or equal to 10 min. In some embodiments, the one or more reactions are one or more amplification reactions. In some embodiments, the one or more amplification reactions are an isothermal nucleic acid amplification reaction. In some embodiments, the device further comprises a control unit. In some embodiments, the control unit is configured to subject the sample and the substance to the one or more reactions under conditions sufficient to generate the signal subsequent to receipt of the sample. In some embodiments, the control unit is an electronic unit. In some embodiments, the control unit comprises at least one electrode.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is an example of a biomolecule test module and associated dock.

FIG. 2 is an example of a biomolecule test module and associated circular dock.

FIG. 3 is an example of a biomolecule test module.

FIG. 4 is an example biomolecule test module showing example internal electrode placement.

FIG. 5 is an example biomolecule test module comprising an internal heating element.

FIG. 6 is an example of a test cartridge docked in a stand.

FIGS. 7-9 show examples of possible internal components of test cartridge.

FIGS. 10-11 are example top-down views of a safety valve.

FIG. 12 is an example of a side view of a safety valve.

FIGS. 13-14 are an example diagram of actuating a safety valve with a digit.

FIGS. 15-16 are examples of heating elements.

FIG. 17 is an example of a calibration strip.

FIG. 18 is an example of a sample collector.

FIG. 19 is an example of a patterned composite electrode.

FIG. 20 is an example of a flexing composite electrode.

FIG. 21 is an example of patterned composite electrode.

FIGS. 22-23 are examples of patterned composite electrodes.

FIG. 24 is an example of a patterned composite electrode.

FIG. 25 is an example of a composite electrode with a varying depth of an absorptive element.

FIG. 26 is an example of a patterned composite electrode.

FIGS. 27-29 are examples of patterned composite electrodes.

FIG. 30 is an example of an unpatterned composite electrode.

FIGS. 31-32 are examples of a composite electrode.

FIGS. 33-36 are examples of multi-layer composite electrodes.

FIG. 37 is an example multi-layer composite electrode.

FIG. 38 is an example multi-layer composite electrode comprising electrodes adjacent to extended absorptive elements.

FIG. 39 is an example of a composite electrode.

FIG. 40 is an example of a composite electrode.

FIGS. 41-42 are examples of the flexibility of a composite electrode.

FIG. 43 is an example of a spiral composite electrode.

FIGS. 44A and 44B are examples of grid electrode patterning within a composite electrode.

FIG. 45 is an example of an absorptive element comprising a plurality of wells.

FIG. 46 is an example of a plurality of electrodes configured to sit below wells.

FIG. 47 is an example of a diode matrix comprising diodes and traces.

FIG. 48A is an example of an electrode energization scheme.

FIG. 48B is an example of a voltage available from a battery stack.

FIG. 49 is an example of a self-contained flow array.

FIG. 50 is an example of a test cartridge coupled to a stand.

FIGS. 51A-51B are a top down view of the internal fluid paths and components of a cartridge including a bottle.

FIGS. 52A-52B are an example of an optional slider.

FIGS. 53A-53B are an example test cartridge comprising electrodes.

FIGS. 54A-54B are an example test cartridge comprising a heating element.

FIG. 55 is an example of an access port adjacent to guide tracks of a test cartridge.

FIG. 56 is an example of a bottle.

FIG. 57 is an example of an access port adjacent to guide tracks of a test cartridge.

FIG. 58 is an example of a bottle comprising a seal.

FIG. 59 is an example of a sample bottle.

FIG. 60 is an example of a bottle comprising a plurality of chambers.

FIG. 61 is an example of a test bottle.

FIG. 62 is an example of a bottle comprising a plurality of seals.

FIG. 63 is an example side view of an access port comprising a burst element.

FIGS. 64-66 are example side views of some embodiments of access ports comprising burst elements.

FIGS. 67-69 are example top-down views of some embodiments of access ports comprising burst elements.

FIGS. 70-71 are example side and bottom views of a bottle comprising a plurality of interior chambers.

FIG. 72 is an example of an access port comprising a burst blade.

FIGS. 73-74 are examples of a bottom-up view of a seal being burst by a burst blade.

FIGS. 75-76 are examples of different views of burst blades.

FIG. 77 is an example of an electrophoresis bottle.

FIG. 78 is an example bottle comprising electrical elements.

FIG. 79 is an example bottom view of a bottle comprising electrical elements.

FIG. 80 is an example of an access port on a surface of a test cartridge with adjacent electrical element detectors.

FIG. 81 is an example of an electrical element approaching but not yet bridging electrical element detectors.

FIG. 82 is an example of a test module configured for storage of one or more solids.

FIG. 83 is a flow chart of an example method.

FIG. 84 is a flow chart of an example method.

FIG. 85 is a flow chart of an example method.

FIG. 86 is a flow chart of an example method.

FIG. 87 is a flow chart of an example method.

FIG. 88 shows a computer system that is programmed or otherwise configured to implement methods provided herein.

FIG. 89 is an example of a voltage application scheme.

FIG. 90 is an example diagram of actuating a safety valve with a digit.

FIG. 91 is an example of a plurality of composite electrodes comprising a same membrane.

FIG. 92 is an example of a stacked composite electrode.

FIGS. 93A-93C and 94A-94C are examples of protected composite electrodes.

FIG. 95 is an example of an extended membrane composite electrode.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

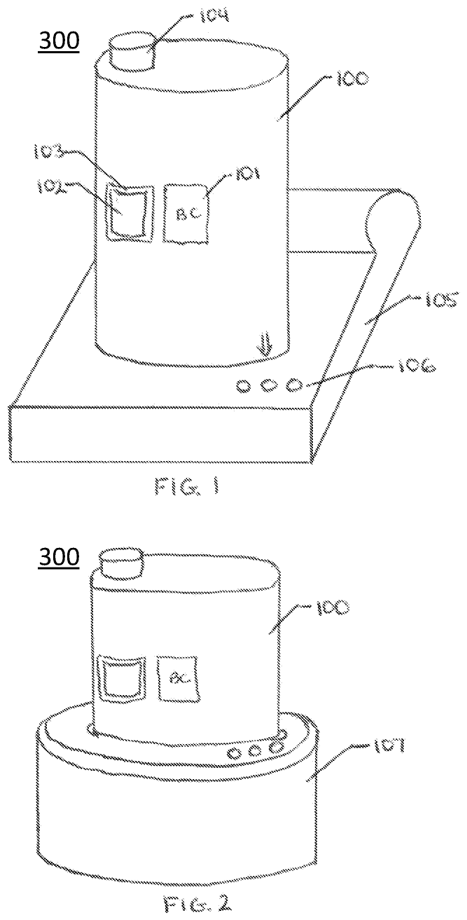

In an aspect, the present disclosure provides methods and devices for assaying a presence or an absence of an analyte. The analyte can be any compound or molecule which may be in question. The analyte may be within a sample. The analyte may be detected directly or indirectly. For example, a presence or absence of an analyte may be determined indirectly by detecting a signal (e.g., an optical signal, an electrical signal, an electrochemical signal, or any combination thereof). The signal may be produced by a detectable label associated with (e.g., linked to, comprised in, or conjugated to) the analyte. As another example, an analyte can be determined directly by, for example, detecting a signal (e.g., an optical signal, an electrical signal, an electrochemical signal, or any combination thereof) resulting from the analyte itself. The analyte may be detected qualitatively and/or quantitatively. The analyte may be related to or indicative of a presence or absence of a physiological condition in a subject. The presence or absence of a physiological condition may be a disease or a condition. The subject may be a mammalian subject. The subject may be a human subject. The analyte may be a biomolecule, for example, a biomarker. The analyte may be a nucleic acid molecule, a protein (e.g., an antibody), an antigen, a chemical (e.g., a toxin), a metal ion (e.g., a heavy metal ion), or the like. A device for assaying a presence or an absence of an analyte may comprise a substrate comprising at least two composite electrodes configured to capture the analyte and detect a signal indicative of the presence or absence of the analyte, upon or subsequent to contact of the analyte with the substrate. The device may be a device as described elsewhere herein. The device may comprise elements as described elsewhere herein such as, for example, FIGS. 1-82, 88-95.

A subject may be an animal, such as a mammal. A subject may be a human or non-human mammal. A subject may be a plant. A subject may be afflicted with a disease or suspected of being afflicted with or having a disease. The subject may not be suspected of being afflicted with or having the disease. The subject may be symptomatic. Alternatively, the subject may be asymptomatic. In some cases, the subject may be treated to alleviate the symptoms of the disease or cure the subject of the disease. A subject may be a patient undergoing treatment by a healthcare provider, such as a doctor. The subject may be a healthcare provider. The subject may be a student, a teacher, a long-term caregiver (e.g., a nursing home employee), a prison guard, or others who work and/or live in close proximity to others.

A sample may be a material that may comprise an analyte. The sample may be suspected of comprising the analyte. A sample may be solid matter (e.g., biological tissue) or may be a fluid (e.g., a biological fluid). In general, a biological fluid can include any fluid associated with living organisms. Non-limiting examples of a samples include blood (or components of blood—e.g., white blood cells, red blood cells, platelets) obtained from any anatomical location (e.g., tissue, circulatory system, bone marrow) of a subject, cells obtained from any anatomical location of a subject, skin, heart, lung, kidney, breath, bone marrow, stool, semen, vaginal fluid, interstitial fluids derived from tumorous tissue, breast, pancreas, cerebral spinal fluid, tissue, throat swab, biopsy, placental fluid, amniotic fluid, liver, muscle, smooth muscle, bladder, gall bladder, colon, intestine, brain, cavity fluids, sputum, pus, micropiota, meconium, breast milk, prostate, esophagus, thyroid, serum, saliva, urine, gastric and digestive fluid, tears, ocular fluids, sweat, mucus, earwax, oil, glandular secretions, spinal fluid, hair, fingernails, skin cells, plasma, nasal swab or nasopharyngeal wash, spinal fluid, cord blood, emphatic fluids, and/or other excretions or body tissues. A sample may be a cell-free sample. Such cell-free sample may include DNA and/or RNA.

A disease or condition may be an abnormal health state of a subject. The terms disease and condition may be used interchangeably herein. The disease or condition may comprise an infectious disease. Examples of infectious disease may include, but are not limited to, acute flaccid myelitis; anaplasmosis; anthrax; babesiosis; botulism; brucellosis; campylobacteriosis; carbapenem-resistant infection; chancroid; chikungunya virus infection; chlamydia; ciguatera; Clostridium difficile infection; Clostridium perfringens; coccidioidomycosis fungal infection; COVID-19; transmissible spongiform encephalopathy; cryptosporidiosis; cyclosporiasis; dengue fever; diphtheria; E. coli infection; eastern equine encephalitis; Ebola hemorrhagic fever; ehrlichiosis; arboviral encephalitis; parainfectious encephalitis; non-polio enterovirus infection; D68 enterovirus infection; giardiasis; glanders; gonococcal infection; granuloma inguinale; haemophilus influenza disease; hantavirus pulmonary syndrome; hemolytic uremic syndrome; hepatitis A; hepatitis B; hepatitis C; hepatitis D; hepatitis E; herpes; herpes zoster; histoplasmosis infection; human immunodeficiency virus; acquired immune deficiency syndrome; human papillomavirus; influenza; legionellosis; leprosy; leptospirosis; listeriosis; Lyme disease; lymphogranuloma venereum infection; malaria; measles; melioidosis; viral meningitis; bacterial meningococcal disease; middle east respiratory syndrome coronavirus; multisystem inflammatory syndrome in children; mumps; norovirus; paralytic shellfish poisoning; pediculosis; pelvic inflammatory disease; pertussis; pneumonic plague; bubonic plague; septicemic plague; pneumococcal disease; poliomyelitis; powassan; psittacosis; pthiriasis; pustular rash diseases; Q-fever; rabies; ricin poisoning; rickettsiosis; rubella; congenital rubella; salmonellosis gastroenteritis; scabies infestation; scombroid; septic shock; severe acute respiratory syndrome; shigellosis gastroenteritis; smallpox; methicillin-resistant staphyloccal infection; staphylococcal food poisoning; vancomycin intermediate staphylococcal infection; vancomycin resistant staphylococcal infection; streptococcal disease; streptococcal toxic-shock syndrome; syphilis; tetanus infection; trichomoniasis; trichonosis infection; tuberculosis; tuberculosis; tularemia; typhoid fever; typhus; bacterial vaginosis; vaping-associated lung injury; varicella; Vibrio cholerae; vibriosis; viral hemorrhagic fever; West Nile virus; yellow fever; yersenia; and Zika virus infection. A disease may be caused by factors originally from an external source, such as infectious disease, or it may be caused by internal dysfunctions, such as autoimmune diseases. A disease can refer to any condition that causes pain, dysfunction, distress, social problems, and/or death to the subject afflicted. A disease may be an acute condition or a chronic condition. A disease may refer to an infectious disease, which may result from the presence of pathogenic microbial agents, including viruses, bacteria, fungi, protozoa, multicellular organisms, and aberrant proteins as prions. A disease may refer to a non-infectious disease, including but not limited to cancer and genetic diseases. In some cases, a disease can be cured. In some cases, a disease cannot be cured.

A nucleic acid molecule may be a molecule comprising one or more nucleotides. The nucleotides may be naturally occurring nucleotides or nucleotide analogs. The nucleotides may be unnatural nucleotides or nucleotide analogs. For example, a nucleotide can be a deoxynucleotide triphosphate (dNTP) or an analog thereof, e.g., a molecule having a plurality of phosphates in a phosphate chain, such as 2, 3, 4, 5, 6, 7, 8, 9, or 10 phosphates. A nucleotide can generally include adenosine (A), cytosine (C), guanine (G), thymine (T) and uracil (U), or variants thereof. A nucleotide can include any subunit that can be incorporated into a growing nucleic acid strand. Such subunit can be an A, C, G, T, or U, or any other subunit that is specific to one or more complementary A, C, G, T or U, or complementary to a purine (e.g., A or G, or variant thereof) or a pyrimidine (e.g., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. A nucleotide may be labeled or unlabeled. A labeled nucleotide may yield a detectable signal, such as an optical, electrical, or electrochemical signal. The nucleic acid molecule may comprise at least about 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 250, 300, 400, 500, 600, 700, 800, 900, 1,000, 5,000, 10,000, 50,000, 100,000 or more nucleotides. The nucleic acid molecule may comprise at most about 100,000, 50,000, 10,000, 5,000, 1,000, 900, 800, 700, 600, 500, 400, 300, 250, 200, 175, 150, 125, 100, 90, 80, 70, 60, 50, 45, 40, 35, 30, 25, 20, 19, 18, 17, 16, 15, 14, 13, 12, 11, 10, 9, 8, 7, 6, 5, 4, 3, or 2 nucleotides. The nucleic acid molecule may be unbound (e.g., in solution). The nucleic acid molecule may be bound (e.g., chemically bonded to a substrate). The nucleic acid molecule may be a deoxyribose nucleic acid (DNA) molecule. The nucleic acid molecule may be a ribose nucleic acid (RNA) molecule. The nucleic acid molecule may be modified. Non-limiting examples of modifications include locked nucleic acids (LNA), peptide nucleic acids (PNAs), methylated bases, biotinylated bases, Fluoro bases, linkable bases (e.g., amino purines), UV cross-linkable bases (e.g., 5-Bromo dU), chain terminator bases (e.g., dideoxy-C), unique binding nucleotides (e.g., iso-dG, iso-dC), Super T® and Super G® nucleotides, and the like.

In another aspect, the present disclosure provides a device comprising a three-dimensional membrane-based substrate comprising at least a first location and a second location. The first location may comprise a first substance specific for a first analyte and may be configured to facilitate generation of a first signal indicative of a presence of the first analyte, upon or subsequent to contact of the first substance with the first analyte. The second location may comprise a second substance specific for a second analyte different from the first analyte and may be configured to facilitate generation of a second signal indicative of a presence of the first analyte, upon or subsequent to contact of the second substance with the second analyte. In some cases, all major components of the device (e.g., the substrate, fluidic channels, fluidic chambers etc.) are paper-based. In some cases, all major components of the device are polymer membrane based (e.g., nylon membrane based). In some cases, all major components of the device are paper or polymer membrane based.

The membrane-based substrate may comprise paper, cellulose, nitro-cellulose, nylon, positively charged nylon, polytetrafluoroethylene, another polymer, or the like, or any combination thereof as the membrane. For example, the membrane can be nylon. The membrane-based substrate may be a portion of a composite electrode. For example, a membrane can have a conductive element applied to the membrane to form a composite electrode. The membrane may have a porosity of at least about 1%, 5%, 10%, 20%, 30%, 40%, 50%, 60%, 70%, 80%, 85%, 90%, 91%, 92%, 93%, 94%, 95%, 96%, 97%, 98%, 99%, 99.5%, 99.9% or more. The membrane may have a porosity of at most about 99.9%, 99.5%, 99%, 98%, 97%, 96%, 95%, 94%, 93%, 92%, 91%, 85%, 80%, 70%, 60%, 50%, 40%, 30%, 20%, 10%, 5%, 1%, or less. The membrane may comprise one or more pore sizes. The one or more pore sizes may be at least about 1, 5, 10, 25, 50, 75, 100, 250, 500, 750, 1,000, 2,500, 5,000, 7,500, 10,000 or more micrometers. The one or more pore sizes may be at most about 10,000, 7,500, 5,000, 2,500, 1,000, 750, 500, 250, 100, 75, 50, 25, 10, 5, 1, or less micrometers. The membrane-based substrate may comprise a conductive element. The conductive element may be a conductive element as described elsewhere herein. The conductive element may be configured to provide electrical attraction, electrical repulsion, holding (e.g., holding a charged or uncharged species in place), mixing (e.g., causing the mixing of two or more species), heating, or the like, or any combination thereof. The membrane may not be a biological membrane (e.g., lipid membrane, bilayer membrane).

The three-dimensional membrane-based substrate may comprise a plurality of locations. The three-dimensional membrane-based substrate may comprise at least about 1, 2, 3, 4, 5, 6, 7, 8, 9, 10, 15, 20, or more locations. The three-dimensional membrane-based substrate may comprise at most about 20, 15, 10, 9, 8, 7, 6, 5, 4, 3, 2, or fewer locations. Each location of the three-dimensional membrane-based substrate may comprise substances specific for different analytes. For example, a membrane-based substrate with 5 locations can have 5 different oligonucleotide probes at each of the 5 locations. In another example, a membrane-based substrate with 3 locations can have one positive control location, one location specific for an analyte, and one negative control location. Multiple locations of the three-dimensional membrane-based substrate may comprise substances specific for a same analyte. For example, two locations on a membrane-based substrate can be specific for the same analyte. In this example, the two locations can reduce a likelihood of a false positive or false negative arising from issues in each of the locations.

The first analyte or the second analyte may comprise nucleic acid molecules. The nucleic acid molecules may be nucleic acid molecules indicative of a condition and/or disease. For example, the nucleic acid molecules may be genomic nucleic acid molecules of a virus. The first substance or the second substance may comprise a primer. The primer may be configured to be a primer for an amplification reaction of the first and/or second analytes. The first analyte or the second analyte may be an antibody, an antibody binding protein, a protein, a macromolecule, a metal ion, or the like.

The substance specific for an analyte may be a substance configured to bind to the analyte. The substance specific for the analyte may be an oligonucleotide, an antibody, a protein, a chelating agent, or the like. For example, the substance can be an oligonucleotide at least partially complimentary to an analyte oligonucleotide. The generation of the signal indicative of a presence of an analyte may be an optical signal, an electrical signal, or a combination thereof. For example, the presence of the analyte can generate a color change and a pH change detectable by an optical detector and an electrical detector, respectively. The optical signal may be a colorimetric signal. The colorimetric signal may comprise a change in optical intensity (e.g., absorptive intensity, fluorescence intensity), a change in optical lifetime (e.g., a change in fluorescence lifetime), or the like, or any combination thereof. The electrical signal may be related to a change in pH, an incorporation of a nucleotide into an oligonucleotide, a presence or absence of an electrical label (e.g., a poly-ionic compound linked to the analyte), or the like, or any combination thereof. The substrate may comprise a plurality of locations each comprising a substance specific for a different analyte. The substances specific for different analytes can be the same type of substance (e.g., all are antibodies, all are oligonucleotides). The substances specific for different analytes may be different types of substances (e.g., a mixture of proteins and oligonucleotides).

The substrate may comprise a structure. The structure may be a structure of the paper. The structure may be a structure of nylon. The structure may be a structure of a composite electrode. The structure may be configured to receive and/or retain a sample. The sample may be suspected of having the first and/or second analytes. The structure may be configured to receive and/or retain the sample by having wells and/or channels. For example, a structure can be configured to receive a sample by the structure comprising a plurality of channels. In this example, the plurality of channels can guide a liquid sample through the structure. The structure may comprise the first and/or the second locations. The structure may comprise the plurality of locations. For example, the substrate can comprise a first and second channel, where the first and second channel are the first and second locations. The substrate may comprise a plurality of structures. The structures may be channels, wells, or the like, or any combination thereof. For example, the substrate can comprise a grid of wells connected by channels. The plurality of the structures may comprise at least a first structure corresponding to the first location and a second structure corresponding to the second location. The first and second structures may be the same type of structure (e.g., both channels). The first and second structures may be different types of structures (e.g., a well and a channel). The type of structure may be chosen depending on the substance specific for an analyte within the structure. For example, a substance that performs better under flow conditions can be in a channel, while a substance that performs worse under flow conditions can be in a well. The first location and the second location can be spatially separated. For example, the first and second locations can be on opposite sides of a wall. In another example, the first and second locations can be wells separated on the substrate. The first location and the second location may overlap. For example, a channel comprising the first location and another channel comprising the second location can cross. In another example, a single channel can comprise the first and second locations as overlapping regions along the length of the channel.

The structure may be a microstructure. The microstructure may have a dimension (e.g., height, width, depth, cross section, etc.) of at least about 1, 5, 10, 25, 50, 75, 100, 250, 500, 750, 1,000, 2,500, 5,000, 7,500, 10,000 or more micrometers. The microstructure may have a dimension of at most about 10,000, 7,500, 5,000, 2,500, 1,000, 750, 500, 250, 100, 75, 50, 25, 10, 5, 1, or less micrometers. The microstructure may be a microfluidic structure. The microstructure may be a structure configured to allow fluid flow without the aid of a subject applied force (e.g., by gravity, by wetting forces). The structure may be a well. The structure may be a fluidic channel. The fluidic channel may be configured to facilitate detection of one or more analytes. For example, the fluidic channel can comprise a substance specific for an analyte. For example, the fluidic channel can comprise binding probes for detection of antibodies. The one or more analytes may be a different analyte. The structure may comprise a plurality of locations each comprising a substance specific for a different analyte.

Individual locations of the plurality of locations may be independently addressable, individually addressable, individually controllable, asynchronously addressable, asynchronously controllable, or any combination thereof. For example, the individual locations can be configured such that each generates a separate signal from the other locations. For example, a substrate with three locations can generate three different optical signals related to the presence or absence of three analytes. In another example, each individually addressable location can be operatively coupled to different electrodes. The plurality of locations may comprise at least about 1, 2, 3, 4, 5, 6, 7, 8, 9, 10, 15, 20, or more locations. The plurality of locations may comprise at most about 20, 15, 10, 9, 8, 7, 6, 5, 4, 3, 2, or fewer locations.

The substrate may comprise one or more additional locations. The one or more additional locations may be configured to act as a positive control or a negative control. For example, an additional location can comprise a substance specific to an analyte known to be in a sample, thus acting as a positive control. In another example, another additional location can be configured without a substance specific to any analytes suspected of being in the sample, thus acting as a negative control.

The device may further comprise one or more additional membrane-based substrates. The one or more additional membrane-based substrates may be membrane-based substrates as described elsewhere herein. For example, the membrane-based substrate can be a composite electrode. The one or more additional substrates may be configured to detect different analytes from the substrate. For example, a first membrane-based substrate can be configured to detect a first and second analyte, and a second membrane-based substrate can be configured to detect a third and fourth analyte. Detecting different analytes on different substrates can increase the number of analytes that can be detected and may permit for modular detection schemes. For example, a subject can choose what analytes to test for by choosing different substrates. The one or more additional substrates may be configured to detect the same analytes from the substrate. For example, both a first and second membrane-based substrate can be configured to each detect a first and second analyte. Detecting the same analytes on two different substrates may result in an increased accuracy of the detection.

The device may further comprise a detection unit in sensory communication with the first location and/or the second location. The detection unit may be in sensory communication with a plurality of locations. The detection unit may be a unit configured to detect a presence or absence of a signal (e.g., a first signal, a second signal) from one or more locations (e.g., the first and/or second locations). The detection of the presence or absence of the signal may determine the presence or absence of one or more analytes (e.g., the first and/or second analytes). The detection unit may be an optical detection unit. The optical detection unit may comprise a camera, a charge coupled device (CCD) sensor, a complimentary metal-oxide-semiconductor (CMOS) sensor, a photodiode, or the like, or any combination thereof. The camera may be a part of a mobile device of a subject (e.g., a smartphone camera). For example, a subject can use a camera integral to their smartphone to image a location of the membrane-based substrate to detect an optical signal generated on the membrane-based substrate. The detection unit may be an electrical detection unit. The electrical detection unit may be one or more electrodes. The one or more electrodes may be at least about 1, 2, 3, 4, 5, 6, 7, 8, 9, 10, or more electrodes. The one or more electrodes may be at most about 10, 9, 8, 7, 6, 5, 4, 3, 2, or fewer electrodes. The one or more electrodes may comprise carbon-based electrodes (e.g., graphite electrodes, glassy carbon electrodes, carbon paper electrodes, graphitized paper electrodes), metal electrodes (e.g., gold, silver, platinum, copper, etc.), semiconductor electrodes, or the like, or any combination thereof. The detection unit may comprise one or more detection units. The one or more detection units may be at least about 1, 2, 3, 4, 5, 6, 7, 8, 9, 10, 15, 20, or more detection units. The one or more detection units may be at most about 20, 15, 10, 9, 8, 7, 6, 5, 4, 3, 2, or fewer detection units. Each of the one or more detection units may be configured to detect the signal generated from each of the locations of the membrane-based substrate. For example, a location configured to generate an optical signal can be operatively coupled to an optical detector (e.g., a camera) while a location configured to generate an electrical signal can be operatively coupled to an electrode.

The detection unit may be a personal device of a subject. The personal device of the subject may be a smartphone (e.g., an iPhone®, an Android® phone), a tablet (e.g., an iPad®, an Android® tablet), a wearable device (e.g., a smartwatch), a computer (e.g., a laptop computer, a desktop computer with a webcam), or the like. For example, a camera module of a subject's tablet can be used as an optical detection unit. In another example, the subject can plug electrical leads into the user's computer so the computer can be used as an electrical detection unit. The subject may be a person suspected of having a condition and/or a disease. For example, the subject can be a person suspected of having a viral infection. The presence or absence of the first analyte and/or second analyte may be indicative of the subject having the condition or disease. For example, the presence of the analytes can be indicative of the subject having a viral infection. The subject may be a person not suspected of having a condition and/or a disease. For example, the subject can be an apparently health person who is screening for a disease to ensure that they are not an asymptomatic carrier.

The device may be a portable device. The device may have a weight of at least about 0.1, 0.5, 1, 1.5, 2, 2.5, 3, 3.5, 4, 4.5, 5, 5.5, 6, 6.5, 7, 7.5, 8, 8.5, 9, 9.5, 10 or more pounds. The device may have a weight of at most about 10, 9.5, 9, 8.5, 8, 7.5, 7, 6.5, 6, 5.5, 5, 4.5, 4, 3.5, 3, 2.5, 2, 1.5, 1, 0.5, 0.1, or less pounds. The device may have a footprint (e.g., an area) of at least about 0.1, 0.5, 1, 1.5, 2, 2.5, 3, 3.5, 4, 4.5, 5, 5.5, 6, 6.5, 7, 7.5, 8, 8.5, 9, 9.5, 10 or more square feet. The device may have a footprint of at most about 10, 9.5, 9, 8.5, 8, 7.5, 7, 6.5, 6, 5.5, 5, 4.5, 4, 3.5, 3, 2.5, 2, 1.5, 1, 0.5, 0.1, or less square feet. The device may comprise a power source. The power source may be a battery, a capacitor, a solar panel, or the like, or any combination thereof. The device may comprise an energy input port. The energy input port may be a wall power connection (e.g., a 120V power cable, a 240V power cable). The device may comprise all components essential for operation. For example, the device can comprise the electrical storage, processing capacity, and reagents needed to operate.

The device may comprise one or more bottles configured to contain one or more reagents. The bottles may be configured to be disposable (e.g., the bottles are configured for one-time use). The bottles may be reagent bottles as described elsewhere herein. The one or more reagents may be reagents for an amplification reaction, a washing process, a detection process, or the like, or any combination thereof. The reagents for the amplification reaction may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The reagents for the amplification reaction may be recombinase polymerase amplification (RPA) amplification reagents. The reagents for the amplification reaction may be helicase dependent isothermal amplification reagents. The reagents for the washing process may comprise organic solvents (e.g., alcohols, ethers, esters), water, salts, ionic species (e.g., salts), buffers, or the like, or any combination thereof. The reagents for the detection process may comprise a binding dye, a fluorescent dye, a labeled nucleic acid strand, a labeled primer, a labeled nucleotide, or the like, or any combination thereof. The reagents may be one or more gasses (e.g., air, inert gas, etc.). The gasses may be used as drying reagents.

Reagents may be components for performing a reaction. Non limiting examples of reagents may include one or more monoclonal antibodies, polyclonal antibodies, antigens, oligonucleotides, model organisms (e.g., cell lines), enzymes, peroxidases, proteins, polymerases, ligases, nucleases, serums, nucleotides, stains, acids, bases, buffers, crowding agents, chemical reagents, or the like.

The one or more bottles may comprise a feedback module. The feedback module may be as described elsewhere herein. The feedback module may be configured to provide information regarding the application of the one or more reagents from the one or more bottles. For example, a bottle can be labeled by the feedback module, thus identifying the contents of the bottle to the device. In another example, the feedback module can be configured to provide information regarding how far the bottle has been inserted into the device. The feedback module may be an electronic feedback module. The electronic module may comprise a conductive module, a capacitive module, a resistive module, or the like, or any combination thereof. For example, the bottle can have a strip of copper along the top of the bottle, where the strip of copper is configured to bridge two reporter electrodes when the bottle is fully inserted, thus enabling a processor coupled to the reporter electrodes to sense that the bottle has been fully inserted. In another example, the bottle can have a resistor on the top of the bottle, where the reporter electrodes are configured to permit the measurement of the resistance of the resistor to identify which bottle has been inserted. The first analyte and/or the second analyte may comprise nucleic acid molecules, antibodies, proteins, or the like, or any combination thereof. The first substance and/or the second substance may comprise a primer, an antigen, a chelating molecule, a cofactor, or the like, or any combination thereof.

In another aspect, the present disclosure provides a method comprising directing a sample suspected of having a first analyte or a second analyte different from the first analyte to a device. The device may comprise a three-dimensional membrane-based substrate. The membrane-based substrate may comprise at least a first location and a second location. The first location may comprise a first substance specific for the first analyte and may be configured to facilitate generation of a first signal indicative of a presence of the first analyte, upon or subsequent to contact of the first substance with the first analyte. The second location may comprise a second substance specific for the second analyte and may be configured to facilitate generation of a second signal indicative of a presence of the second analyte, upon or subsequent to contact of the second substance with the second analyte. A presence or absence of the first signal or the second signal may be detected from the first location or the second location, upon or subsequent to the sample being directed to the device. A presence or absence of the first analyte or the second analyte may be determined, based on the presence or absence of the first signal or the second signal.

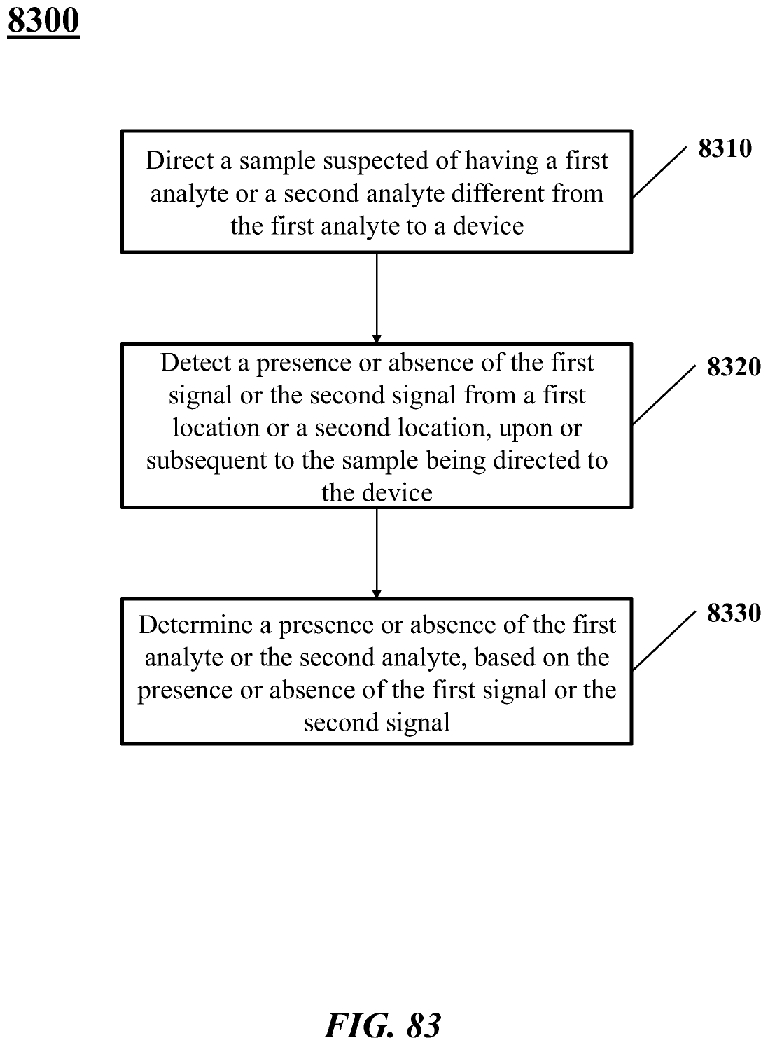

FIG. 83 is a flow chart of an example method 8300. In an operation 8310, the method 8300 may comprise directing a sample suspected of having a first analyte or a second analyte different from the first analyte to a device. The device may be a device as described elsewhere herein. The device may comprise a three-dimensional membrane-based substrate. The membrane-based substrate may comprise at least a first location and a second location. The first location may comprise a first substance specific for the first analyte and may be configured to facilitate generation of a first signal indicative of a presence of the first analyte, upon or subsequent to contact of the first substance with the first analyte. The second location may comprise a second substance specific for the second analyte and may be configured to facilitate generation of a second signal indicative of a presence of the second analyte, upon or subsequent to contact of the second substance with the second analyte. The first analyte and the second analyte may be the same analyte. For example, the analyte can be detected in a same way but at spatially separated locations. The first signal from the first analyte and the second signal from the second analyte may be a same type of signal. For example, the first signal and the second signal can both be a purple colorimetric signal created by the application of Horseradish peroxidase, but generated at spatially separated locations.

The sample may be from a subject suspected of having a disease and/or a condition. For example, the sample may be from a subject showing symptoms of a disease. The sample may be from a subject who is not suspected of having a disease and/or condition. For example, the sample may be from a subject who is asymptomatic for a disease. The sample may be from a subject suspected of being infected with a pathogen. The pathogen may be related to the condition and/or disease. For example, the pathogen can be a cause of a disease. The first analyte and/or the second analyte may comprise nucleic acid molecules. The nucleic acid molecules may comprise a ribonucleic acid (RNA) molecule of a disease and/or condition. The RNA molecule may be an RNA of severe acute respirator syndrome coronavirus-19 (SARS-CoV-2) or a fragment thereof. The pathogen may be SARS-CoV-2.

The sample may have a volume of at least about 0.01, 0.05, 0.1, 0.2, 0.3, 0.4, 0.5, 0.6, 0.7, 0.8, 0.9, 1.0, 1.1, 1.2, 1.3, 1.4, 1.5, 2.0, 2.5, 5.0, 7.5, 10.0, or more milliliters. The sample may have a volume of at most about 10.0, 7.5, 5.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.05, 0.01, or less milliliters. The sample may comprise a pH buffer. The sample may not comprise a pH buffer. For example, the sample can be a fluid taken directly from the body of a subject. The sample may be mixed with a reaction mixture comprising a pH buffer. For example, a saliva sample from a subject can be mixed with a reaction mixture comprising a pH buffer after the sample is introduced to the device. The sample may comprise a fluid from the body of a subject. The sample may comprise saliva, blood, or the like, or any combination thereof. The sample may be taken from a subject using a swab. The swab may comprise a breakable head as described elsewhere herein. The sample may be at a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The sample may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The sample may have a pH as defined by any two of the proceeding values. For example, the sample may have a pH from about 4.0 to about 6.0.

In another operation 8320, the method 8300 may comprise detecting a presence or absence of the first signal or the second signal from the first location or the second location, upon or subsequent to the sample being directed to the device. The first signal or the second signal may comprise a signal increase relative to a baseline. For example, the signals can be an increase in color from a pH indicator. The first signal or the second signal may comprise a signal decrease relative to a baseline. For example, the signal can be a decrease in the absorption of a colorimetric dye due to the presence of an amplified nucleic acid. The detecting the presence or absence of the first signal and/or the second signal may occur at a temperature of at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The detecting the presence or absence of the first signal and/or the second signal may occur at a temperature of at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius. The detecting the presence or absence of the first signal and/or the second signal may occur at a temperature range as defined by any two of the proceeding values. For example, the detecting can occur at a temperature range of 35-40 degrees Celsius.

The first signal and/or the second signal comprise colorimetric signals. The colorimetric signals may be related to an absorption (e.g., an absorption intensity, an absorption wavelength), a fluorescence (e.g., a fluorescence intensity, a fluorescence wavelength, a fluorescence lifetime), a plasmonic property, or the like, or any combination thereof. For example, the colorimetric signal can be a change in intensity and color of a colorimetric reagent on the substrate upon amplification of an oligonucleotide analyte. The colorimetric signal may be generated by a commercially available kit, such as, for example, the New England BioLabs® colorimetric LAMP assay kit. The detecting the first signal and/or the second signal may comprise detecting a color calibration panel disposed adjacent to the first location and/or the second location. The color calibration panel may comprise one or more calibration markers. The calibration markers may be color fields on the device that can be used to calibrate a detector to the imaging conditions of the device. The color fields may be black, white, one or more greyscale colors, a color of a positive result, a color of a negative result, primary colors, secondary colors, one or more of the ColorChecker® calibration colors, or the like, or any combination thereof. For example, a color calibration panel can comprise a black panel, a white panel, a 50% grey panel, the color of a positive result, and the color of a negative result can be placed adjacent to the first location and the second location. In this example, the detected image of the colorimetric signal can be white balanced, and the color of the calibration panel can be compared to that of the first and second locations to improve the accuracy of the detection of the presence or absence of the signal.

The first signal and/or the second signal may comprise an electrical signal. The electrical signal may be detected as described elsewhere herein. The electrical signal may be measured at or across an electrode. The electrode may be adjacent to, embedded in, surrounding, or the like, the substrate. The electrical signal may be a resistance, impedance, capacitance, or the like, a change thereof, or any combination thereof. For example, the electrical signal can be a change in a capacitance of the substrate. The electrical signal may be related to a change in a solution comprising the first and/or second analyte. The change may be a change in pH, conductivity, ionic strength, dielectric constant, or the like, or any combination thereof. For example, an electrode adjacent to the substrate can measure a change in pH in a solution adjacent to the substrate caused by polymerase activity. One of the first signal or the second signal may be a colorimetric signal and the other of the first signal or the second signal may be an electrical signal. For example, an electrode can measure an electrical signal corresponding to a change in pH while a camera records a colorimetric change. In another example, the electrode can measure a change in conductivity across an electrode resulting from an oxidation or reduction reaction occurring at or near the electrode.

In another operation 8330, the method 8300 may comprise determining a presence or absence of the first analyte or the second analyte. The determining may be based on the presence or absence of the first signal or the second signal. For example, a sample not comprising the first analyte but comprising the second analyte can generate the second signal but not the first signal, thus indicating the presence of the second analyte and the absence of the first analyte. The method may further comprise outputting a report that identifies the presence or absence of the first analyte and/or the second analyte. The report may comprise one or more color codes indicative of the first analyte and/or the second analyte. For example, the report can be a green screen displayed on the screen of a device of the subject to indicate the absence of the analytes. In another example, the report can be a red screen displayed on the screen of a device of the subject to indicate the possibility of the presence of at least one of the analytes. In this example, the red screen can indicate that the subject may have a disease and requires further testing. The method may further comprise displaying the report on a personal device of the subject. For example, the report can be displayed on a screen of a smartphone. The subject may be a subject from which the sample is obtained. For example, the subject can be a person who swabbed themselves, performed the method, and received the results on their device. The subject may be a healthcare provider. For example, the healthcare provider can perform the method on a patient and can receive the results on a lab computer. The personal device may be a mobile device as described elsewhere herein. The mobile device may comprise a light. The light may be configured to illuminate a colorimetric signal. For example, a smartphone comprising a light emitting diode (LED) and a camera can illuminate the colorimetric signal with the LED and image the colorimetric signal with the camera. In this example, the LED can provide brighter and more consistent lighting than ambient light. The personal device may be in communication with the device. For example, the personal device and the device can be in wireless communication to alert the subject to review the results of the method. In another example, the device can be wired into the personal device, and information from the device including the presence or absence of the signals can be transmitted via the wires to the personal device for display.

In another aspect, the present disclosure provides a device. The device may comprise two or more fluidic chambers, a fluidic channel between and in fluidic communication with the two or more fluidic chambers, and a valve disposed within the fluidic channel. The valve may comprise a chamber. The chamber may be compressible or expandable. The chamber may be configured to regulate fluid flow between the two or more fluidic chambers upon an actuation of the chamber.

The two or more fluidic chambers may be chambers of a device as described elsewhere herein. The two or more fluidic chambers may be a reagent chamber and a reaction chamber. The fluidic channel may be a microfluidic channel (e.g., a fluidic channel with a diameter of less than about 1 millimeter). The fluidic channel may have a diameter of greater than about 1 millimeter. The fluidic channel may be configured to permit a passive flow of fluid within the fluidic channel.

The valve may be impassable by a fluid before actuation. For example, the valve may initially be impermeable to a liquid. The chamber may be a plastic encased bubble. The chamber may be a thin metal foil encased bubble. The encased bubble may be a plastic encased air bubble. For example, the chamber may be a gas surrounded by a plastic film. In another example, the chamber may be filled with an inert gas. The chamber may be pressurized with gas. For example, the chamber can be filled with gas at above atmospheric pressure. The chamber may be filled with non-pressurized gas. For example, the chamber can be filled with gas at ambient pressure. The chamber may be inflated. For example, the chamber can be inflated with a gas. The chamber may be configured to not break upon the application of the pressure to only one side of the chamber. For example, an application of pressure to the fluidic channel on one side of the chamber can be insufficient to break the chamber. In this example, only one side of the chamber may rupture, leaving the other side intact and the chamber unbroken. In another example, the combined thickness of both sides of the chamber can be sufficient to prevent the breaking of both of the sides when pressure is applied to the fluidic channel on one side of the chamber. The valve may comprise a pressure breakable seal. The pressure breakable seal may be a thin wall of the chamber. For example, the chamber can comprise thin walls that are configured to burst under pressure. The pressure breakable seal may be a weakened portion of the chamber. For example, a portion of the walls of the chamber can be thinner than the rest of the walls. In this example, the thinner portion can be configured to break when a pressure is applied to them.

The actuation may comprise an application of a pressure to the chamber. The pressure may be a positive pressure. For example, the pressure may be an application of force to the chamber. The pressure may be applied manually. For example, the pressure can be applied by a subject using their fingers. In another example, a subject can press on an upper member which in turn presses on the chamber. The pressure may be applied automatically. For example, a driver motor can move a rod to apply the pressure. In another example, gas can be automatically flowed into the chamber to increase the pressure.

A thickness of a membrane wall of the valve may be less than a thickness of a wall of the fluidic channel. For example, a membrane wall can have a thickness of 0.5 millimeters, while the wall of the fluidic channel can have a thickness of 3 millimeters. The membrane wall of the valve may be configured to have a lower burst pressure than the wall of the fluidic channel. For example, a thinner membrane wall can burst at a lower pressure than a thicker fluidic channel. The thickness of the membrane wall may be at least about 0.01, 0.05, 0.1, 0.2, 0.3, 0.4, 0.5, 0.6, 0.7, 0.8, 0.9, 1.0, 2.5, 5.0, or more millimeters. The thickness of the membrane wall may be at most about 5.0, 2.5, 1.0, 0.9, 0.8, 0.7, 0.6, 0.5, 0.4, 0.3, 0.2, 0.1, 0.05, 0.01, or less millimeters.

The device may be a single tube device as described elsewhere herein. The device may be a cartridge device as described elsewhere herein. The cartridge or the single tube device may comprise a plurality of valves. The plurality of valves may connect a plurality of fluidic chambers. The device may be a portable device as described elsewhere herein. The device may comprise the valve between a sample chamber and a reagent chamber. For example, a valve can be positioned between a chamber comprising reagents for an amplification reaction and a reaction chamber adjacent to an electrode. The sample chamber and the reagent chamber may be affixed to a rigid support as described elsewhere herein.

In another aspect, the present disclosure provides a method. The method may comprise directing a fluid to a device. The device may comprise two or more fluidic chambers, a fluidic channel between and in fluidic communication with the two or more fluidic chambers, and a valve disposed within the fluidic channel. The valve may comprise a chamber. The chamber may be compressible or expandable. The chamber may be configured to regulate fluid flow between the two or more fluidic chambers upon an actuation of the chamber. The chamber may be actuated to regulate fluid flow between the two or more fluidic chambers.

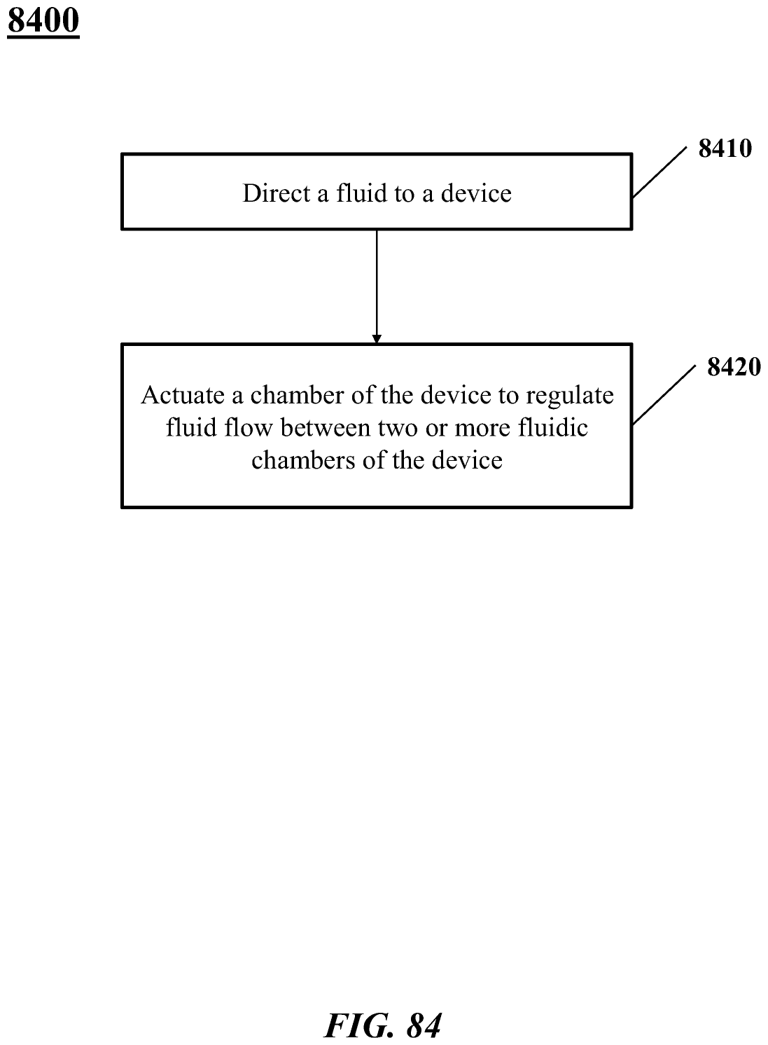

FIG. 84 is a flow chart of an example method 8400. In an operation 8410, the method 8400 may comprise directing a fluid to a device. The device may comprise two or more fluidic chambers, a fluidic channel between and in fluidic communication with the two or more fluidic chambers, and a valve disposed within the fluidic channel. The valve may comprise a chamber. The chamber may be compressible or expandable. The chamber may be configured to regulate fluid flow between the two or more fluidic chambers upon an actuation of the chamber. The device may be a device as described elsewhere herein. The fluid may be a sample as described elsewhere herein (e.g., saliva, blood).

In another operation 8420, the method 8400 may comprise actuating the chamber of the device to regulate fluid flow between the two or more fluidic chambers of the device. The regulating fluid flow may comprise bruising the chamber. For example, the chamber can be broken to permit fluid flow between the two or more fluidic chambers through the broken chamber. The actuation may comprise applying a pressure to the chamber. The pressure may be applied to a center of the chamber. For example, a subject can press their finger in the center of the chamber to burst the walls of the chamber. The chamber may be configured not to burst if the pressure is not applied to the center of the chamber. The pressure may be a pressure of at least about 0.01, 0.05, 0.1, 0.2, 0.3, 0.4, 0.5, 0.6, 0.7, 0.8, 0.9, 1, 2, 3, 4, 5, 6, 7, 8, 9, 10, or more megapascals. The pressure may be a pressure of at most about 10, 9, 8, 7, 6, 5, 4, 3, 2, 1, 0.9, 0.8, 0.7, 0.6, 0.5, 0.4, 0.3, 0.2, 0.1, 0.05, 0.01, or less megapascals.

The operation 8420 may further comprise actuating the chamber a second time. The actuating the chamber the second time may be applying the pressure a second time to the chamber. For example, a subject can press the chamber a second time with their finger. The actuating the chamber the second time may comprise applying a pressure to fully break the chamber. For example, after a first application of pressure that broke one side of the chamber, a subject can press the chamber again to break the other side of the chamber.

In another aspect, the present disclosure provides a device. The device may comprise an inlet configured to receive a sample. The device may comprise a fluidic channel in fluidic connection with the inlet and a fluidic region downstream of the inlet. The fluidic channel may be configured to passively or actively flow the sample from the inlet to the fluidic region upon receipt of the sample. The device may comprise at least one electrode adjacent to and operably coupled to the fluidic region. The at least one electrode may be configured to enrich for one or more analytes from the sample in the fluidic region. The at least one electrode may be configured to subject the one or more analytes to one or more reactions under conditions sufficient to yield a signal indicative of a presence or absence of an analyte among the one or more analytes. The at least one electrode may be configured to detect the signal from the fluidic region, thereby determining the presence or absence of the analyte in the sample. The at least one electrode may comprise a plurality of electrodes (e.g., greater than or equal to 2, 3, 4, 5, 6, 7, 8, 9, 10 electrodes, or more). Individually electrodes of the plurality of electrodes may be configured, individually or collectively, to perform one or more of the above. In some examples, the plurality of electrodes comprises multiple subsets of electrodes, which may be operably coupled to different subregions of the fluidic region. Each individual subset of electrodes may be configured to perform a different function (e.g., analyte enrichment, reaction, detection, determination etc.). The inlet may be an access port as described herein. The inlet port may be a sample bottle as described elsewhere herein.

The at least one electrode may be configured to apply an electric field of at least about 0.001, 0.005, 0.01, 0.05, 0.1, 0.5, 1, 1.5, 2, 2.5, 3, 3.5, 4, 4.5, 5, 5.5, 6, 6.5, 7, 7.5, 8, 8.5, 9, 9.5, 10, 25, 50, 75, 100, or more volts to concentrate the one or more analytes. The at least one electrode may be configured to apply an electric field of at most about 100, 75, 50, 25, 10, 9.5, 9, 8.5, 8, 7.5, 7, 6.5, 6, 5.5, 5, 4.5, 4, 3.5, 3, 2.5, 2, 1.5, 1, 0.5, 0.1, 0.05, 0.01, 0.005, 0.001, or less volts to concentrate the one or more analytes. The at least one electrode may be configured to apply an electric field as defined by any two of the proceeding values. For example, the at least one electrode can apply an electric field of 3-5 volts. The electric field may be configured to concentrate the one or more analytes by attracting charged analytes towards the electrode. For example, a positive electrode can attract negatively charged nucleic acid molecules towards the electrode. The one or more electrodes may be configured to enrich a membrane-based substrate as described elsewhere herein with the one or more analytes. For example, an electrode can be positioned on the other side of a membrane-based substrate from a solution containing the one or more analytes. In this example, the electrode can attract the one or more analytes towards the electrode, and the membrane-based substrate can capture the one or more analytes. The at least one electrode may be configured to cycle electrical current. For example, the at least one electrode can be positively biased and subsequently negatively biased. In another example, the at least one electrode can cycle between positive and negative bias in accordance with instructions provided by a processor. The cycling of the electrical current may be configured to improve an enrichment of the one or more analytes. For example, an electrode can be positively biased to attract negatively charged nucleic acids into the membrane-based substrate where the nucleic acids hybridize to capture primers contained within the substrate. In this example, subsequently to the nucleic acids hybridizing to the capture primers, the electrode can be negatively biased to repel unbound nucleic acid molecules and aid in a washing of the substrate. In this example, the substrate can have a lower number of non-hybridized nucleic acid molecules due to the cycling of the electrode.

The at least one electrode may be configured to subject the one or more analytes to a temperature of at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The at least one electrode may be configured to subject the one or more analytes to a temperature of at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius. The at least one electrode may be configured to subject the one or more analytes to a temperature as defined by any two of the proceeding values. For example, the at least one electrode can be configured to subject the one or more analytes to a temperature of about 30-70 degrees Celsius. In another example, the at least one electrode can be configured to subject the one or more analytes to a temperature of about 35-40 degrees Celsius. The electrode may be configured as a resistive heater. For example, the electrode can heat up when current is flowed through it. The electrode may be configured to heat the sample while enriching for the one or more analytes. For example, the electrode can heat the sample while attracting the analytes from the sample.

The device may comprise a heating element. The heating element may be different from the electrode. The heating element may be configured to subject the one or more analytes to a temperature of at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The heating element may be configured to subject the one or more analytes to a temperature of at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius. The heating element may be configured to subject the one or more analytes to a temperature as defined by any two of the proceeding values. For example, the heating element can be configured to subject the one or more analytes to a temperature of about 30-70 degrees Celsius. In another example, the heating element can be configured to subject the one or more analytes to a temperature of about 35-40 degrees Celsius.

The at least one electrode may comprise two or more electrodes. The two or more electrodes may be configured in a concentric arrangement. For example, a first electrode can be surrounded by a second electrode. The concentric arrangement may be a target pattern. The two or more concentric electrodes may be configured to be sequentially charged to sequentially enrich for the one or more analytes. For example, for a device comprising three electrodes, the outermost electrode can be charged to enrich the area of the outermost electrode with analytes. In this example, the middle electrode can then be charged to concentrate the analytes into the smaller area of the middle electrode, and subsequently the center electrode can be charged to concentrate the analytes into the smaller area of the center electrode. In this example, the sequential charging of the concentric electrodes can increase the final concentration of analytes at the center electrode. The two or more electrodes may be configured in a grid, a series of lines, any geometric shape (e.g., a triangle, a square), or the like. For example, a plurality of electrodes can be configured as a hexagonal grid of electrodes.

The at least one electrode may comprise a metal (e.g., gold, silver, copper, platinum), an alloy (e.g., brass), a semiconductor (e.g., silicon), carbon (e.g., graphite, glassy carbon, graphene), an organic conductor (e.g., a polymer electrode), or the like, or any combination thereof. The at least one electrode may comprise conductive carbon paper. For example, a paper substrate can be impregnated with graphite, thus generating a conductive paper electrode. In another example, a graphite sheet can be an electrode. The at least one electrode may be a membrane-based substrate as described elsewhere herein. The at least one electrode may be a composite electrode as described elsewhere herein.

The device may be a portable device as described elsewhere herein. The device may further comprise one or more bottles configured to contain one or more reagents as described elsewhere herein. The bottles may be configured to be disposable (e.g., the bottles are configured for one-time use). The bottles may be reagent bottles as described elsewhere herein. The one or more reagents may be reagents for an amplification reaction, a washing process, a detection process, or the like, or any combination thereof. The reagents for the amplification reaction may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The reagents for the washing process may comprise organic solvents (e.g., alcohols, ethers, esters), water, salts, ionic species (e.g., salts, chaotropic salts), buffers, or the like, or any combination thereof. The reagents for the amplification reaction may be recombinase polymerase amplification (RPA) amplification reagents. The reagents for the amplification reaction may be helicase dependent isothermal amplification reagents. The reagents for the detection process may comprise a binding dye, a fluorescent dye, a labeled nucleic acid strand, or the like, or any combination thereof. The reagents may be one or more gasses (e.g., air, inert gas, etc.). The gasses may be used as drying reagents. The reagents may comprise one or more chaotropic reagents. The chaotropic reagents may be configured to aid in a binding of an analyte molecule to a membrane. For example, the chaotropic reagents can be configured to increase a binding between a nucleic acid and a cellulose membrane. Examples of chaotropic reagents include, but are not limited to, guanidinium HCl, lithium acetate, magnesium chloride, sodium dodecyl sulfate (SDS), urea, butanol, lithium perchlorate, phenol, isopropanol, thiourea, ethanol, surfactants, or the like, or any combination thereof. The chaotropic reagents may comprise positive amino acid buffers.

The one or more bottles may comprise a feedback module. The feedback module may be as described elsewhere herein. The feedback module may be configured to provide information regarding the application of the one or more reagents from the one or more bottles. For example, a bottle can be labeled by the feedback module, thus identifying the contents of the bottle to the device. In another example, the feedback module can be configured to provide information regarding how far the bottle has been inserted into the device. The feedback module may be an electronic feedback module. The electronic module may comprise a conductive module, a capacitive module, a resistive module, or the like, or any combination thereof. For example, the bottle can have a strip of copper along the top of the bottle, where the strip of copper is configured to bridge two reporter electrodes when the bottle is fully inserted, thus enabling a processor coupled to the reporter electrodes to sense that the bottle has been fully inserted. In another example, the bottle can have a resistor on the top of the bottle, where the reporter electrodes are configured to permit the measurement of the resistance of the resistor to identify which bottle has been inserted. The first analyte and/or the second analyte may comprise nucleic acid molecules, antibodies, proteins, or the like, or any combination thereof. The first substance and/or the second substance may comprise a primer, an antigen, a chelating molecule, a cofactor, or the like, or any combination thereof.

In another aspect, the present disclosure provides a method. The method may comprise directing a sample to a device. The device may comprise an inlet configured to receive a sample. The device may comprise a fluidic channel in fluidic connection with the inlet and a fluidic region downstream of the inlet. The fluidic channel may be configured to passively or actively flow the sample from the inlet to the fluidic region upon receipt of the sample. The device may comprise at least one electrode adjacent to and operably coupled to the fluidic region. The at least one electrode may be configured to enrich for one or more analytes from the sample in the fluidic region. The at least one electrode may be configured to subject the one or more analytes to one or more reactions under conditions sufficient to yield a signal indicative of a presence or absence of an analyte among the one or more analytes. The at least one electrode may be configured to detect the signal from the fluidic region, thereby determining the presence or absence of the analyte in the sample. The sample may be passively or actively flowed from the inlet to the fluidic region via the fluidic channel. The at least one electrode may be used to, in the fluidic region, enrich for one or more analytes from the sample. The at least one electrode may be used to, in the fluidic region, subject the one or more analytes to one or more reactions under conditions sufficient to yield a signal indicative or a presence or an absence of an analyte among the one or more analytes. The signal may be detected from the fluidic region using the at least one electrode, thereby determining the presence or absence of the analyte in the sample.

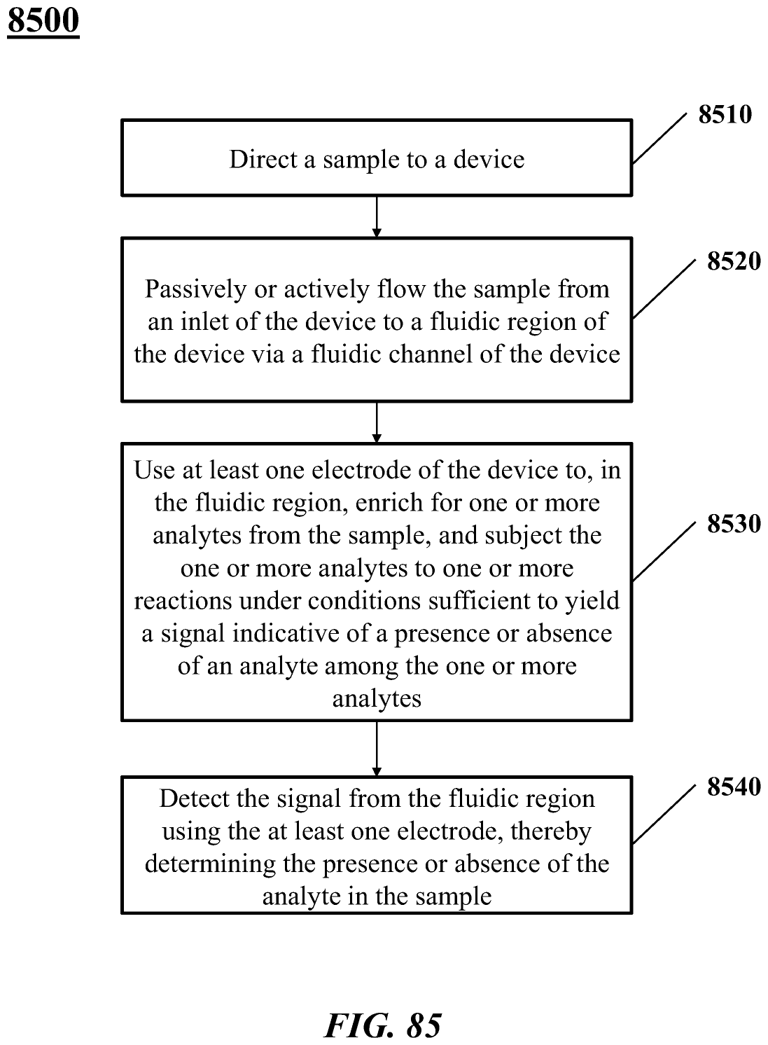

FIG. 85 is a flow chart of an example method 8500. In an operation 8510, the method 8500 may comprise directing a sample to a device. The device may be a device as described elsewhere herein. The device may comprise an inlet configured to receive a sample. The device may comprise a fluidic channel in fluidic connection with the inlet and a fluidic region downstream of the inlet. The fluidic channel may be configured to passively or actively flow the sample from the inlet to the fluidic region upon receipt of the sample. The device may comprise at least one electrode adjacent to and operably coupled to the fluidic region. The at least one electrode may be configured to enrich for one or more analytes from the sample in the fluidic region. The at least one electrode may be configured to subject the one or more analytes to one or more reactions under conditions sufficient to yield a signal indicative of a presence or absence of an analyte among the one or more analytes. The at least one electrode may be configured to detect the signal from the fluidic region, thereby determining the presence or absence of the analyte in the sample.

In another operation 8520, the method 8500 may comprise passively or actively flowing the sample from the inlet of the device to the fluidic region of the device via the fluidic channel of the device. The passive flowing may comprise flowing without any input from a subject. The passive flowing may use gravity, wetting forces, or the like, to effect the flowing. For example, the subject can place the sample in the inlet of the device and allow the sample to flow into the fluidic region by gravity. The active flowing may comprise input from a subject or from the device. For example, the subject can squeeze a soft chamber holding the sample to push the sample through the fluidic channel. In another example, the subject can shake the device. The device can effect the flow of the sample by, for example, pressurizing the inlet, flowing additional liquid to carry along the sample, provide an electric field to attract the sample to the fluidic region, or the like, or any combination thereof. The fluidic channel may comprise a membrane-based substrate. The membrane-based substrate can direct the sample towards the fluidic region by wetting effects. The membrane-based substrate may be a membrane-based substrate as described elsewhere herein (e.g., a paper-based substrate, a nylon-based substrate, a composite paper and/or nylon-based substrate, etc.). The terms membrane and absorptive element may be used interchangeably herein.

In another operation 8530, the method 8500 may comprise using the least one electrode of the device to, in the fluidic region, enrich for one or more analytes from the sample, and subject the one or more analytes to one or more reactions under conditions sufficient to yield a signal indicative of a presence or absence of an analyte among the one or more analytes.

The enriching for the one or more analytes may comprise applying an electric field to the sample. The subjecting the one or more analytes to the reaction may comprise using the at least one electrode to heat the one or more analytes. The electric field may be configured to attract or repel charged analytes of the one or more analytes. For example, a negatively charged electrode can attract and enrich protons generated by a polymerization reaction. The electric field may be an electric field as described elsewhere herein. The electric field may be configured to lyse one or more cells of the sample. The lysing of the one or more cells may expose the one or more analytes to the solution. For example, a cell comprising a nucleic acid of interest can be lysed to place the nucleic acid into solution where it can be concentrated by the at least one electrode. The lysing of the cell may occur due to an electric field applied across the cell. The electric field may be pulsed. For example, an electric field can be rapidly pulsed on and off to lyse the cell. In another example, the polarity of the electric field can be rapidly reversed to lyse the cell. The method may further comprise pulsing an electrical current through the at least one electrode to mix the one or more analytes with one or more reagents. For example, a pulsed electrode can generate a pulsed electric field, which can move charged analytes and reagents in a solution to mix the analytes and reagents. The at least one electrode may comprise a plurality of electrodes. Individual electrodes of the plurality of electrodes may be configured to, individually or collectively, (1) enrich for one or more analytes from the sample in the fluidic region, (2) subject the one or more analytes to one or more reactions under conditions sufficient to yield a signal indicative of a presence or absence of a analyte among the one or more analytes, and/or (3) detect the signal from the fluidic region.

The subjecting the one or more analytes to the reaction may comprise using the at least one electrode to heat the one or more analytes. The heating may be heating as described elsewhere herein. The one or more analytes may comprise one or more nucleic acids. The heating may be sufficient to perform an isothermal amplification reaction of the one or more nucleic acids. For example, the electrode can be configured to hold the solution comprising the nucleic acids and amplification reagents at a temperature sufficient to activate the amplification reagents and amplify the nucleic acids. The isothermal amplification reaction may improve the sensitivity, specificity, and/or the accuracy of the detecting of operation 8540. The improvement may be due to an increase in the number of nucleic acid molecules in the sample, thus increasing the amount of signal generated. The presence of the amplification may offer benefit over non-amplified methods.

In another operation 8540, the method 8500 may comprise detecting the signal from the fluidic region using the at least one electrode, thereby determining the presence or absence of the analyte in the sample. The determining of the presence or absence of the analyte may be performed at an accuracy, sensitivity, and/or specificity of at least about 10%, 20%, 30%, 40%, 50%, 60%, 70%, 80%, 85%, 90%, 91%, 92%, 93%, 94%, 95%, 96%, 97%, 98%, 99%, 99.5%, 99.9% or more. The determining of the presence or absence of the analyte may be performed at an accuracy, sensitivity, and/or specificity of at most about 99.9%, 99.5%, 99%, 98%, 97%, 96%, 95%, 94%, 93%, 92%, 91%, 85%, 80%, 70%, 60%, 50%, 40%, 30%, 20%, 10%, or less.

The detecting the signals may comprise detecting an electrical signal. The electrical detection may be electrical detection as described elsewhere herein. The electrical signal may be generated by a change in a pH, an ionic strength, a packing, a result of an oxidation or reduction reaction in close proximity to an electrode, or the like, or any combination thereof, of the sample. The change in the pH, ionic strength, packing, or the like, may be due to an amplification of one or more nucleic acids within the sample. For example, an increase in the pH of a sample due to the action of a polymerase amplifying a nucleic acid can be detected by detecting a change in the electrical properties of the at least one electrode. The detecting may comprise both detecting an optical signal and an electrical signal as described elsewhere herein.

The detecting the signal may comprise detecting an optical signal. The detecting the optical signal may be as described elsewhere herein. For example, the detecting the optical signal may be detecting a colorimetric optical signal. The colorimetric optical signal may be generated by a change in a pH of the sample, a binding of a dye to at least a portion of the sample or a derivative thereof, a releasing of a colorimetric indicator due to a reaction of at least a portion of the sample (e.g., releasing a bound dye due to a binding event of a nucleic acid to a primer), or the like, or any combination thereof. The change in the pH of the sample may generate a change in a color of a pH indicator. The pH indicator may be, for example, bromophenol blue, Congo red, methyl orange, bromocresol green, resazurin, 4-phenylazo-1-napthylamine, ethyl red, Resorcin blue, bromocresol purple, p-nitrophenol, phenol red, neutral red, curcumin, metacresol purple, thymol blue, phenolphthalein, or the like. The colorimetric signal may be a colorimetric optical signal generated by an enzymatic oxidation or reduction of a substrate. For example, an enzyme can be activated by a reaction comprising an analyte, and the enzyme can degrade a dye to decrease a colorimetric signal. The enzymatic oxidation or reduction of the substrate may comprise the use of a horseradish peroxidase, an alkaline phosphatase, a nanoparticle (e.g., a metal nanoparticle, a semiconductor nanoparticle, a metal oxide nanoparticle, an organic nanoparticle, etc.), a metal containing compound (e.g., a catalyst comprising a metal ion), an artificial enzyme, or the like, or any combination thereof. For example, a horseradish peroxidase can be activated by an amplification reaction, and the horseradish peroxidase can perform an oxidation of a chromogenic substance to generate a colorimetric signal.

The device may further comprise a color standard positioned to be viewable when performing the detecting as described elsewhere herein. The color standard may be used to calibrate for a color of the optical signal. For example, for a device using Congo red as the pH indicator, the color standard can comprise blocks of color corresponding to the endpoint colors of Congo red (e.g., blue and red). In this example, the image of the fluidic region can be calibrated to improve the fidelity of the image to the actual color of the fluidic region. The presence of the color standard can permit calibration of the colors in an image taken of the fluidic region. The calibration can increase the accuracy, sensitivity, and/or specificity of the detecting.

In an aspect, the present disclosure provides a device. The device may comprise a membrane-based substrate. The membrane-based substrate may comprise a recess configured to receive and retain a sample having a volume of less than or equal to about 5 microliters (μL). The membrane-based substrate may comprise a surface comprising a substance specific for an analyte and configured to facilitate generation of a signal indicative of a presence or absence of the analyte in the sample, upon or subsequent to contact of the sample with the surface. The sample may have a volume of at least about 1, 2, 3, 4, 5, 6, 7, 8, 9, 10, 25, 50, 75, 100, 250, 500, 750, 1,000, 1,250, 1,500, 1,750, 2,000, 2,500, 3,000, 4,000, 5,000, or more microliters. The sample may have a volume of at most about 5,000, 4,000, 3,000, 2,500, 2,000, 1,750, 1,500, 1,250, 1,000, 750, 500, 250, 100, 75, 50, 25, 10, 9, 8, 7, 6, 5, 4, 3, 2, 1, or less microliters. The substance specific for an analyte may be a nucleotide, an oligonucleotide (e.g., a primer), an antibody, an antigen, a protein, a chelating agent, a macromolecule, a chemical species (e.g., a chemical configured to react with the analyte), or the like, or any combination thereof.

The signal may be an optical signal as described elsewhere herein. The optical signal may be detected as described elsewhere herein. For example, the detecting the optical signal may be detecting a colorimetric optical signal. The colorimetric optical signal may be generated by a change in a pH of the sample, a binding of a dye to at least a portion of the sample or a derivative thereof, a releasing of a colorimetric indicator due to a reaction of at least a portion of the sample (e.g., releasing a bound dye due to a binding event of a nucleic acid to a primer), or the like, or any combination thereof. The change in the pH of the sample may generate a change in a color of a pH indicator. The pH indicator may be, for example, bromophenol blue, Congo red, methyl orange, bromocresol green, resazurin, 4-phenylazo-1-napthylamine, ethyl red, Resorcin blue, bromocresol purple, p-nitrophenol, phenol red, neutral red, curcumin, metacresol purple, thymol blue, phenolphthalein, or the like. The colorimetric signal may be a colorimetric optical signal generated by an enzymatic oxidation or reduction of a substrate. For example, an enzyme can be activated by a reaction comprising an analyte, and the enzyme can degrade a dye to decrease a colorimetric signal. The enzymatic oxidation or reduction of the substrate may comprise the use of a horseradish peroxidase, an alkaline phosphatase, a nanoparticle (e.g., a metal nanoparticle, a semiconductor nanoparticle, a metal oxide nanoparticle, an organic nanoparticle, etc.), a metal containing compound (e.g., a catalyst comprising a metal ion), an artificial enzyme, or the like, or any combination thereof. For example, a horseradish peroxidase can be activated by an amplification reaction, and the horseradish peroxidase can perform an oxidation of a chromogenic substance to generate a colorimetric signal.

The signal may be an electrical signal as described elsewhere herein. For example, the electrical signal may be related to or generated by a change in a pH of the sample. The electrical signal may be detected by an electrical detection as described elsewhere herein. The electrical signal may be generated by a change in a pH, an ionic strength, a packing, a result of an oxidation or reduction reaction in close proximity to an electrode, or the like, or any combination thereof, of the sample. The change in the pH, ionic strength, packing, or the like, may be due to an amplification of one or more nucleic acids within the sample. For example, an increase in the pH of a sample due to the action of a polymerase amplifying a nucleic acid can be detected by detecting a change in the electrical properties of the at least one electrode. The detecting may comprise both detecting an optical signal and an electrical signal as described elsewhere herein. In some embodiments, the electrical signal may be related to a change in a conductivity across an electrode resulting from an oxidation or reduction reaction.

The membrane-based substrate may comprise a plurality of recesses. The membrane-based substrate may be a three-dimensional membrane-based substrate as described elsewhere herein (e.g., a paper-based substrate, a nylon-based substrate, a composite electrode, etc.). For example, the membrane-based substrate can comprise a plurality of channels within the membrane-based substrate configured to have the sample flow through them. In another example, the membrane-based substrate can comprise a plurality of wells. The membrane-based substrate may be impregnated with the substance specific for the analyte. For example, the membrane-based substrate can have primers embedded within the paper. Each of the plurality of recesses may comprise a surface each comprising a substance specific for a different analyte. For example, adjacent channels of the membrane-based substrate can have different primers complimentary to different nucleic acid molecules. The plurality of recesses may be configured to facilitate generation of a signal indicative of a presence or absence of the different analytes in the sample, upon or subsequent to contact of the sample with the plurality of recesses. For example, each recess of the membrane-based substrate can have a different primer complimentary to a different nucleic acid analyte such that when the different nucleic acid analytes flow into the recesses, each recess generates a signal indicative of the particular nucleic acid analyte the recess is configured to be specific for.

The device may comprise a plurality of membrane-based substrates. The plurality of membrane-based substrates may be in an array. The array may be a grid (e.g., a square grid, a hexagonal grid), a line, a shape (e.g., three substrates are organized in a triangle), amorphous (e.g., placed adjacent without long range order), or the like. Each membrane-based substrate of the membrane-based substrates may comprise a surface comprising substances specific for a different analyte of a plurality of analytes. For example, a device comprising three circular paper substrates organized in a line can have each circular paper substrate comprise a different substance specific for a different analyte. In another example, each row of a grid of paper substrates can be specific for a particular analyte, and each column can be specific for different analytes. In this example, the number of paper substrates in the columns of the grid can be related to the number of different analytes that can be tested, and the number of paper substrates in the rows of the grid can be related to the error checking redundancy of the test.

The device may comprise at least one electrode. The at least one electrode may be at least one electrode as described elsewhere herein. The at least one electrode may be configured to detect the signal as described elsewhere herein. The at least one electrode may comprise two or more electrodes. The two or more electrodes may be configured in a concentric arrangement. For example, a first electrode can be surrounded by a second electrode. The concentric arrangement may be a target pattern. The two or more concentric electrodes may be configured to be sequentially charged to sequentially enrich for the one or more analytes. For example, for a device comprising three electrodes, the outermost electrode can be charged to enrich the area of the outermost electrode with analytes. In this example, the middle electrode can then be charged to concentrate the analytes into the smaller area of the middle electrode, and subsequently the center electrode can be charged to concentrate the analytes into the smaller area of the center electrode. In this example, the sequential charging of the concentric electrodes can increase the final concentration of analytes at the center electrode. The two or more electrodes may be configured in a grid, a series of lines, any geometric shape (e.g., a triangle, a square), or the like. For example, a plurality of electrodes can be configured as a hexagonal grid of electrodes. The at least one electrode may comprise a metal (e.g., gold, silver, copper, platinum), an alloy (e.g., brass), a semiconductor (e.g., silicon), carbon (e.g., graphite, glassy carbon, graphene), an organic conductor (e.g., a polymer electrode), or the like, or any combination thereof. The at least one electrode may comprise conductive carbon paper. For example, a paper substrate can be impregnated with graphite, thus generating a conductive paper electrode. In another example, a graphite sheet can be an electrode.

In another aspect, the present disclosure provides a method. The method may comprise directing a sample to a device. The device may comprise a membrane-based substrate. The membrane-based substrate may comprise a recess configured to receive and retain a sample having a volume of less than or equal to about 5 microliters (μL). The membrane-based substrate may comprise a surface comprising a substance specific for an analyte and configured to facilitate generation of a signal indicative of a presence or absence of the analyte in the sample, upon or subsequent to contact of the sample with the surface. The signal may be detected from the surface upon or subsequent to contact of the sample with the surface, thereby determining a presence or absence of the analyte in the sample. The sample may have a volume of at least about 1, 2, 3, 4, 5, 6, 7, 8, 9, 10, 25, 50, 75, 100, 250, 500, 750, 1,000, 1,250, 1,500, 1,750, 2,000, 2,500, 3,000, 4,000, 5,000, or more microliters. The sample may have a volume of at most about 5,000, 4,000, 3,000, 2,500, 2,000, 1,750, 1,500, 1,250, 1,000, 750, 500, 250, 100, 75, 50, 25, 10, 9, 8, 7, 6, 5, 4, 3, 2, 1, or less microliters.

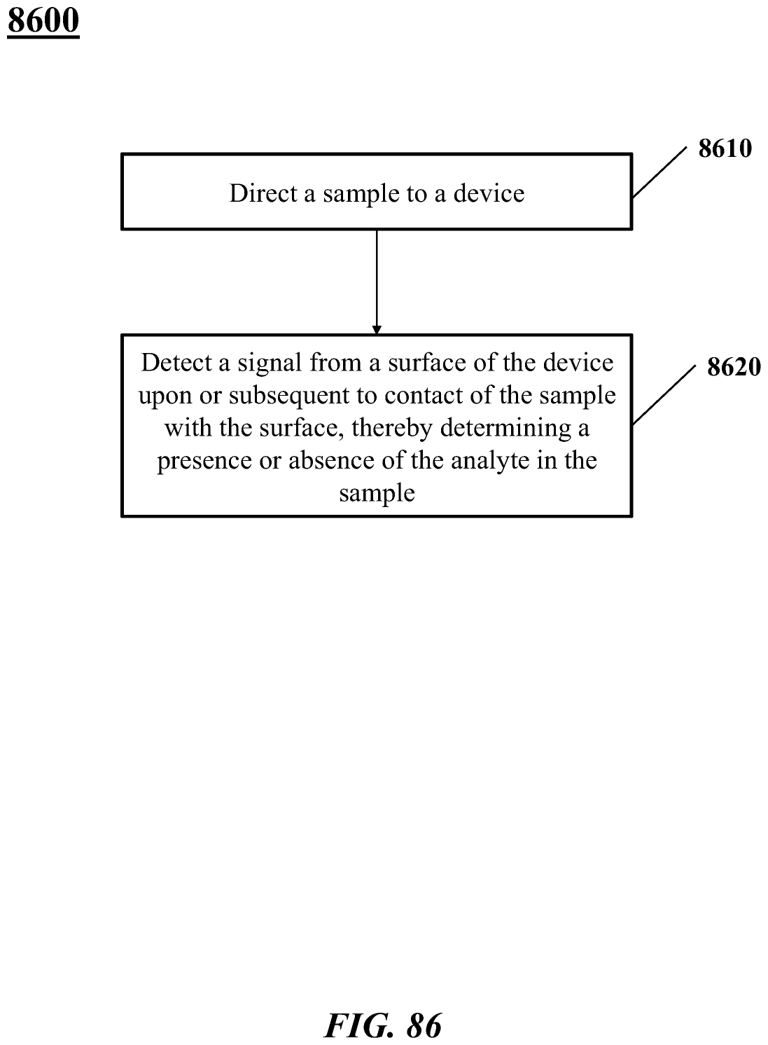

FIG. 86 is a flow chart of an example method 8600. In an operation 8610, the method 8600 may comprise directing a sample to a device. The device may comprise a membrane-based substrate. The membrane-based substrate may comprise a recess configured to receive and retain the sample having a volume of less than or equal to about 5 microliters (μL). The membrane-based substrate may comprise a surface comprising a substance specific for an analyte and configured to facilitate generation of a signal indicative of a presence or absence of the analyte in the sample, upon or subsequent to contact of the sample with the surface. The signal may be detected from the surface upon or subsequent to contact of the sample with the surface, thereby determining a presence or absence of the analyte in the sample. The sample may have a volume of at least about 1, 2, 3, 4, 5, 6, 7, 8, 9, 10, 25, 50, 75, 100, 250, 500, 750, 1,000, 1,250, 1,500, 1,750, 2,000, 2,500, 3,000, 4,000, 5,000, or more microliters. The sample may have a volume of at most about 5,000, 4,000, 3,000, 2,500, 2,000, 1,750, 1,500, 1,250, 1,000, 750, 500, 250, 100, 75, 50, 25, 10, 9, 8, 7, 6, 5, 4, 3, 2, 1, or less microliters.

The sample may be from a subject suspected of having a disease and/or a condition. For example, the sample may be from a subject showing symptoms of a disease. The sample may be from a subject who is not suspected of having a disease and/or condition. For example, the sample may be from a subject who is asymptomatic for a disease. The sample may be from a subject suspected of being infected with a pathogen. The pathogen may be related to the condition and/or disease. For example, the pathogen can be a cause of a disease. The analyte may comprise nucleic acid molecules. The nucleic acid molecules may comprise a ribonucleic acid (RNA) molecule of a disease and/or condition. The RNA molecule may be an RNA of severe acute respirator syndrome coronavirus-19 (SARS-CoV-2) or a fragment thereof. The pathogen may be SARS-CoV-2.

In another operation 8620, the method 8600 may comprise detecting a signal from the surface of the device upon or subsequent to contact of the sample with the surface, thereby determining a presence or absence of the analyte in the sample.

The signal may be an optical signal as described elsewhere herein. The optical signal may be detected as described elsewhere herein. For example, the detecting the optical signal may be detecting a colorimetric optical signal. The colorimetric optical signal may be generated by a change in a pH of the sample, a binding of a dye to at least a portion of the sample or a derivative thereof, a releasing of a colorimetric indicator due to a reaction of at least a portion of the sample (e.g., releasing a bound dye due to a binding event of a nucleic acid to a primer), or the like, or any combination thereof. The change in the pH of the sample may generate a change in a color of a pH indicator. The pH indicator may be, for example, bromophenol blue, Congo red, methyl orange, bromocresol green, resazurin, 4-phenylazo-1-napthylamine, ethyl red, Resorcin blue, bromocresol purple, p-nitrophenol, phenol red, neutral red, curcumin, metacresol purple, thymol blue, phenolphthalein, or the like. The colorimetric signal may be a colorimetric optical signal generated by an enzymatic oxidation or reduction of a substrate. For example, an enzyme can be activated by a reaction comprising an analyte, and the enzyme can degrade a dye to decrease a colorimetric signal. The enzymatic oxidation or reduction of the substrate may comprise the use of a horseradish peroxidase, an alkaline phosphatase, a nanoparticle (e.g., a metal nanoparticle, a semiconductor nanoparticle, a metal oxide nanoparticle, an organic nanoparticle, etc.), a metal containing compound (e.g., a catalyst comprising a metal ion), an artificial enzyme, or the like, or any combination thereof. For example, a horseradish peroxidase can be activated by an amplification reaction, and the horseradish peroxidase can perform an oxidation of a chromogenic substance to generate a colorimetric signal.

The signal may be an electrical signal as described elsewhere herein. For example, the electrical signal may be related to or generated by a change in a pH of the sample. The electrical signal may be detected by an electrical detection as described elsewhere herein. The electrical signal may be generated by a change in a pH, an ionic strength, a packing, a result of an oxidation or reduction reaction in close proximity to an electrode, or the like, or any combination thereof, of the sample. The change in the pH, ionic strength, packing, or the like, may be due to an amplification of one or more nucleic acids within the sample. For example, an increase in the pH of a sample due to the action of a polymerase amplifying a nucleic acid can be detected by detecting a change in the electrical properties of the at least one electrode. The detecting may comprise both detecting an optical signal and an electrical signal as described elsewhere herein. In some embodiments, the electrical signal may be related to a change in a conductivity across an electrode resulting from an oxidation or reduction reaction.

In another aspect, the present disclosure provides a device. The device may comprise a membrane-based substrate. The membrane-based substrate may comprise a recess configured to receive and retain a sample having a volume of less than or equal to about 2 milliliters and/or a substance specific for an analyte and configured to facilitate generation of a signal indicative of a presence or absence of the analyte in the sample, upon or subsequent to contact of the sample with the surface. The device may comprise a control unit configured to subject the sample and the substance to one or more reactions under conditions sufficient to generate the signal within 5 minutes (min) subsequent to receipt of the sample. The sample may have a volume of at least about 0.01, 0.05, 0.1, 0.2, 0.3, 0.4, 0.5, 0.6, 0.7, 0.8, 0.9, 1.0, 1.1, 1.2, 1.3, 1.4, 1.5, 2.0, 2.5, 5.0, 7.5, 10.0, or more milliliters. The sample may have a volume of at most about 10.0, 7.5, 5.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.05, 0.01, or less milliliters. The signal may be generated within at least about 0.1 seconds (s), 1 s, 5 s, 10 s, 30 s, 1 minute (m), 5 m, 10 m, 15 m, 30 m, 1 hour (h), 2 h, 3 h, 4 h, 5 h, 6 h, 7 h, 8 h, 9 h, 10 h, 12 h, 18 h, 24 h, 48 h, 72 h, 96 h, or more. The signal may be generated within at most about 96 h, 72 h, 48 h, 24 h, 18 h, 12 h, 10 h, 9 h, 8 h, 7 h, 6 h, 5 h, 4 h, 3 h, 2 h, 1 h, 30 m, 15 m, 10 m, 5 m, 1 m, 30 s, 10 s, 5 s, 1 s, 0.1 s, or less. The sample may be at a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The sample may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The sample may have a pH as defined by any two of the proceeding values. For example, the sample may have a pH from about 4.0 to about 6.0.

The signal may be an optical signal as described elsewhere herein. The optical signal may be detected as described elsewhere herein. For example, the detecting the optical signal may be detecting a colorimetric optical signal. The colorimetric optical signal may be generated by a change in a pH of the sample, a binding of a dye to at least a portion of the sample or a derivative thereof, a releasing of a colorimetric indicator due to a reaction of at least a portion of the sample (e.g., releasing a bound dye due to a binding event of a nucleic acid to a primer), or the like, or any combination thereof. The change in the pH of the sample may generate a change in a color of a pH indicator. The pH indicator may be, for example, bromophenol blue, Congo red, methyl orange, bromocresol green, resazurin, 4-phenylazo-1-napthylamine, ethyl red, Resorcin blue, bromocresol purple, p-nitrophenol, phenol red, neutral red, curcumin, metacresol purple, thymol blue, phenolphthalein, or the like. The colorimetric signal may be a colorimetric optical signal generated by an enzymatic oxidation or reduction of a substrate. For example, an enzyme can be activated by a reaction comprising an analyte, and the enzyme can degrade a dye to decrease a colorimetric signal. The enzymatic oxidation or reduction of the substrate may comprise the use of a horseradish peroxidase, an alkaline phosphatase, a nanoparticle (e.g., a metal nanoparticle, a semiconductor nanoparticle, a metal oxide nanoparticle, an organic nanoparticle, etc.), a metal containing compound (e.g., a catalyst comprising a metal ion), an artificial enzyme, or the like, or any combination thereof. For example, a horseradish peroxidase can be activated by an amplification reaction, and the horseradish peroxidase can perform an oxidation of a chromogenic substance to generate a colorimetric signal.

The signal may be an electrical signal as described elsewhere herein. For example, the electrical signal may be related to or generated by a change in a pH of the sample. The electrical signal may be detected by an electrical detection as described elsewhere herein. The electrical signal may be generated by a change in a pH, an ionic strength, a result of an oxidation or reduction reaction in close proximity to an electrode, a packing, or the like, or any combination thereof, of the sample. The change in the pH, ionic strength, packing, or the like, may be due to an amplification of one or more nucleic acids within the sample. For example, an increase in the pH of a sample due to the action of a polymerase amplifying a nucleic acid can be detected by detecting a change in the electrical properties of the at least one electrode. The detecting may comprise both detecting an optical signal and an electrical signal as described elsewhere herein. In some embodiments, the electrical signal may be related to a change in a conductivity across an electrode resulting from an oxidation or reduction reaction.

The membrane-based substrate may comprise a plurality of recesses. The membrane-based substrate may be a three-dimensional membrane-based substrate as described elsewhere herein (e.g., a paper-based substrate, a nylon-based substrate, a composite electrode, etc.). For example, the membrane-based substrate can comprise a plurality of channels within the membrane-based substrate configured to have the sample flow through them. In another example, the membrane-based substrate can comprise a plurality of wells. The membrane-based substrate may be impregnated with the substance specific for the analyte. For example, the membrane-based substrate can have primers embedded within the paper. Each of the plurality of recesses may comprise a surface each comprising a substance specific for a different analyte. For example, adjacent channels of the membrane-based substrate can have different primers complimentary to different nucleic acid molecules. The plurality of recesses may be configured to facilitate generation of a signal indicative of a presence or absence of the different analytes in the sample, upon or subsequent to contact of the sample with the plurality of recesses. For example, each recess of the membrane-based substrate can have a different primer complimentary to a different nucleic acid analyte such that when the different nucleic acid analytes flow into the recesses, each recess generates a signal indicative of the particular nucleic acid analyte the recess is configured to be specific for.

The device may comprise a plurality of membrane-based substrates. The plurality of membrane-based substrates may be in an array. The array may be a grid (e.g., a square grid, a hexagonal grid), a line, a shape (e.g., three substrates are organized in a triangle), amorphous (e.g., placed adjacent without long range order), or the like. Each membrane-based substrate of the membrane-based substrates may comprise a surface comprising substances specific for a different analyte of a plurality of analytes. For example, a device comprising three circular paper substrates organized in a line can have each circular paper substrate comprise a different substance specific for a different analyte. In another example, each row of a grid of paper substrates can be specific for a particular analyte, and each column can be specific for different analytes. In this example, the number of paper substrates in the columns of the grid can be related to the number of different analytes that can be tested, and the number of paper substrates in the rows of the grid can be related to the error checking redundancy of the test.

The device may comprise at least one electrode. The at least one electrode may be at least one electrode as described elsewhere herein. The at least one electrode may be configured to detect the signal as described elsewhere herein. The at least one electrode may comprise two or more electrodes. The two or more electrodes may be configured in a concentric arrangement. For example, a first electrode can be surrounded by a second electrode. The concentric arrangement may be a target pattern. The two or more concentric electrodes may be configured to be sequentially charged to sequentially enrich for the one or more analytes. For example, for a device comprising three electrodes, the outermost electrode can be charged to enrich the area of the outermost electrode with analytes. In this example, the middle electrode can then be charged to concentrate the analytes into the smaller area of the middle electrode, and subsequently the center electrode can be charged to concentrate the analytes into the smaller area of the center electrode. In this example, the sequential charging of the concentric electrodes can increase the final concentration of analytes at the center electrode. The two or more electrodes may be configured in a grid, a series of lines, any geometric shape (e.g., a triangle, a square), or the like. For example, a plurality of electrodes can be configured as a hexagonal grid of electrodes. The at least one electrode may comprise a metal (e.g., gold, silver, copper, platinum), an alloy (e.g., brass), a semiconductor (e.g., silicon), carbon (e.g., graphite, glassy carbon, graphene), an organic conductor (e.g., a polymer electrode), or the like, or any combination thereof. The at least one electrode may comprise a conductive carbon membrane. For example, a paper substrate can be impregnated with graphite, thus generating a conductive paper electrode. In another example, a graphite sheet can be an electrode. In another example, a nylon membrane can be impregnated with graphite to generate a conductive nylon membrane electrode.

In another aspect, the present disclosure provides a method. The method may comprise directing (i) a sample having a volume of less than or equal to about 2 milliliters and (ii) a substance to a device. The device may comprise a recess configured to receive and retain the sample and the substance. The substance may be specific for an analyte. The substance may be configured to facilitate generation of a signal indicative of a presence or absence of the analyte in the sample, upon or subsequent to contact of the sample with the surface. The sample and the substance may be subjected to one or more reactions under conditions sufficient to generate the signal. The signal may be detected from the substrate, thereby determining a presence or absence of the analyte in the sample. The directing and the detecting may be separated in time by at least about 0.1 seconds (s), 1 s, 5 s, 10 s, 30 s, 1 minute (m), 5 m, 10 m, 15 m, 30 m, 1 hour (h), 2 h, 3 h, 4 h, 5 h, 6 h, 7 h, 8 h, 9 h, 10 h, 12 h, 18 h, 24 h, 48 h, 72 h, 96 h, or more. The directing and the detecting may be separated in time by at most about 96 h, 72 h, 48 h, 24 h, 18 h, 12 h, 10 h, 9 h, 8 h, 7 h, 6 h, 5 h, 4 h, 3 h, 2 h, 1 h, 30 m, 15 m, 10 m, 5 m, 1 m, 30 s, 10 s, 5 s, 1 s, 0.1 s, or less.

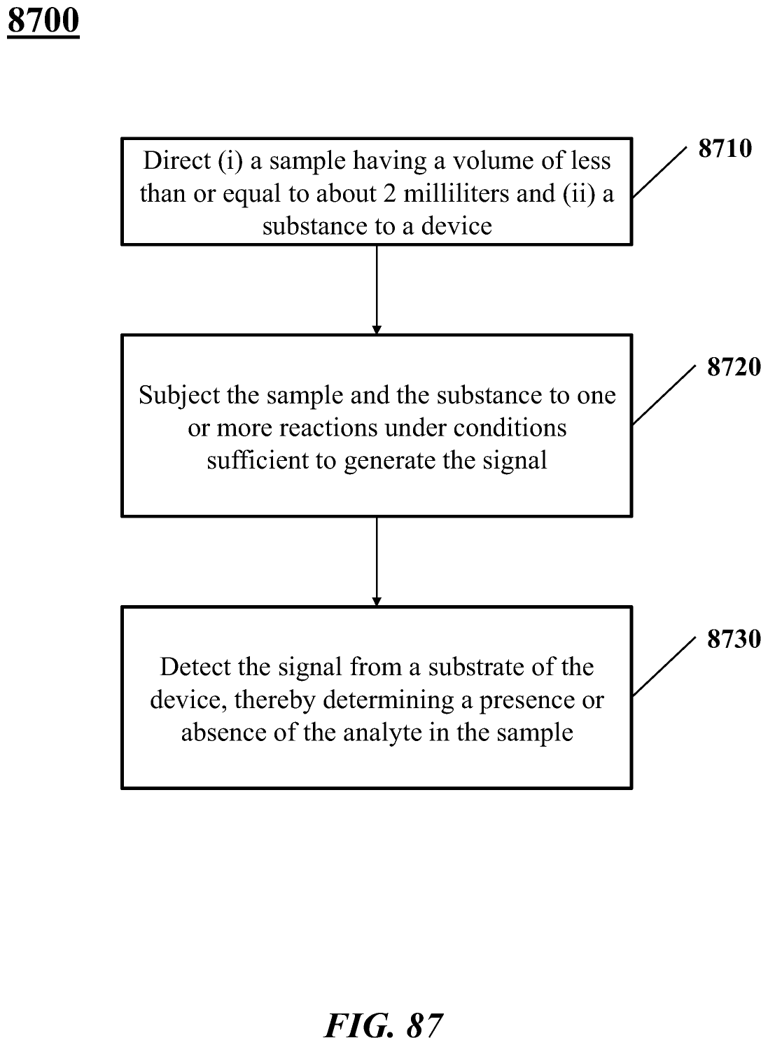

FIG. 87 is an example flow chart of a method 8700. In an operation 8710, the method 8700 may comprise directing (i) a sample having a volume of less than or equal to about 2 milliliters and (ii) a substance to a device. The device may comprise a recess configured to receive and retain said sample and said substance. The substance may be specific for an analyte. The substance may be configured to facilitate generation of a signal indicative of a presence or absence of said analyte in said sample, upon or subsequent to contact of said sample with said surface. The sample may have a volume of at least about 0.01, 0.05, 0.1, 0.2, 0.3, 0.4, 0.5, 0.6, 0.7, 0.8, 0.9, 1.0, 1.1, 1.2, 1.3, 1.4, 1.5, 2.0, 2.5, 5.0, 7.5, 10.0, or more milliliters. The sample may have a volume of at most about 10.0, 7.5, 5.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.05, 0.01, or less milliliters. The sample may be at a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The sample may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The sample may have a pH as defined by any two of the proceeding values. For example, the sample may have a pH from about 4.0 to about 6.0.

In another operation 8720, the method 8700 may comprise subjecting the sample and the substance to one or more reactions under conditions sufficient to generate the signal. The one or more reactions may be one or more amplification reactions as described elsewhere herein. For example, the one or more amplification reactions may be an isothermal nucleic acid amplification reaction. The device may further comprise a control unit. The control unit may be an electronic unit. The electronic unit may comprise at least one electrode as described elsewhere herein. The control unit may comprise a processor as described elsewhere herein. For example, the control unit may comprise a microprocessor configured to control aspects of the device. The control unit may be configured to subject the sample and the substance to the one or more reactions under conditions sufficient to generate the signal subsequent to receipt of the sample. For example, the control unit can direct a heating element to heat the sample to a temperature sufficient for an isothermal amplification of nucleic acids to occur.

In another operation 8730, the method 8700 may comprise detecting the signal from a substrate of the device, thereby determining a presence or absence of the analyte in the sample. Operations 8710 and 8730 may be separated in time by at least about 0.1 seconds (s), 1 s, 5 s, 10 s, 30 s, 1 minute (m), 5 m, 10 m, 15 m, 30 m, 1 hour (h), 2 h, 3 h, 4 h, 5 h, 6 h, 7 h, 8 h, 9 h, 10 h, 12 h, 18 h, 24 h, 48 h, 72 h, 96 h, or more. Operations 8710 and 8730 may be separated in time by at most about 96 h, 72 h, 48 h, 24 h, 18 h, 12 h, 10 h, 9 h, 8 h, 7 h, 6 h, 5 h, 4 h, 3 h, 2 h, 1 h, 30 m, 15 m, 10 m, 5 m, 1 m, 30 s, 10 s, 5 s, 1 s, 0.1 s, or less.

The signal may be an optical signal as described elsewhere herein. The optical signal may be detected as described elsewhere herein. For example, the detecting the optical signal may be detecting a colorimetric optical signal. The colorimetric optical signal may be generated by a change in a pH of the sample, a binding of a dye to at least a portion of the sample or a derivative thereof, a releasing of a colorimetric indicator due to a reaction of at least a portion of the sample (e.g., releasing a bound dye due to a binding event of a nucleic acid to a primer), or the like, or any combination thereof. The change in the pH of the sample may generate a change in a color of a pH indicator. The pH indicator may be, for example, bromophenol blue, Congo red, methyl orange, bromocresol green, resazurin, 4-phenylazo-1-napthylamine, ethyl red, Resorcin blue, bromocresol purple, p-nitrophenol, phenol red, neutral red, curcumin, metacresol purple, thymol blue, phenolphthalein, or the like. The colorimetric signal may be a colorimetric optical signal generated by an enzymatic oxidation or reduction of a substrate. For example, an enzyme can be activated by a reaction comprising an analyte, and the enzyme can degrade a dye to decrease a colorimetric signal. The enzymatic oxidation or reduction of the substrate may comprise the use of a horseradish peroxidase, an alkaline phosphatase, a nanoparticle (e.g., a metal nanoparticle, a semiconductor nanoparticle, a metal oxide nanoparticle, an organic nanoparticle, etc.), a metal containing compound (e.g., a catalyst comprising a metal ion), an artificial enzyme, or the like, or any combination thereof. For example, a horseradish peroxidase can be activated by an amplification reaction, and the horseradish peroxidase can perform an oxidation of a chromogenic substance to generate a colorimetric signal. In another example, horseradish peroxidase may be bound to, or be attracted to and bind to an amplification product. The horseradish peroxidase (HRP) may subsequently oxidize a clear chromogenic substrate such as TMB and generate a colorimetric signal. Alternately the HRP oxidation reaction may lower the resistance between two adjacent electrodes and the resulting change in ionic current can allow the detection of the reaction via changes in resistance, capacitive, or impedance.

The signal may be an electrical signal as described elsewhere herein. For example, the electrical signal may be related to or generated by a change in a pH of the sample. The electrical signal may be detected by an electrical detection as described elsewhere herein. The electrical signal may be generated by a change in a pH, an ionic strength, a packing, a result of an oxidation or reduction reaction in close proximity to an electrode, or the like, or any combination thereof, of the sample. The change in the pH, ionic strength, packing, or the like, may be due to an amplification of one or more nucleic acids within the sample. For example, an increase in the pH of a sample due to the action of a polymerase amplifying a nucleic acid can be detected by detecting a change in the electrical properties of the at least one electrode. The detecting may comprise both detecting an optical signal and an electrical signal as described elsewhere herein. In some embodiments, the electrical signal may be related to a change in a conductivity across an electrode resulting from an oxidation or reduction reaction.

The membrane-based substrate may comprise a plurality of recesses. The membrane-based substrate may be a three-dimensional membrane-based substrate as described elsewhere herein (e.g., a paper-based substrate, a nylon-based substrate, a composite electrode, etc.). For example, the membrane-based substrate can comprise a plurality of channels within the membrane-based substrate configured to have the sample flow through them. In another example, the membrane-based substrate can comprise a plurality of wells. The membrane-based substrate may be impregnated with the substance specific for the analyte. For example, the membrane-based substrate can have primers embedded within the paper. Each of the plurality of recesses may comprise a surface each comprising a substance specific for a different analyte. For example, adjacent channels of the membrane-based substrate can have different primers complimentary to different nucleic acid molecules. The plurality of recesses may be configured to facilitate generation of a signal indicative of a presence or absence of the different analytes in the sample, upon or subsequent to contact of the sample with the plurality of recesses. For example, each recess of the membrane-based substrate can have a different primer complimentary to a different nucleic acid analyte such that when the different nucleic acid analytes flow into the recesses, each recess generates a signal indicative of the particular nucleic acid analyte the recess is configured to be specific for.

The device may comprise a plurality of membrane-based substrates. The plurality of membrane-based substrates may be in an array. The array may be a grid (e.g., a square grid, a hexagonal grid), a line, a shape (e.g., three substrates are organized in a triangle), amorphous (e.g., placed adjacent without long range order), or the like. Each membrane-based substrate of the membrane-based substrates may comprise a surface comprising substances specific for a different analyte of a plurality of analytes. For example, a device comprising three circular paper substrates organized in a line can have each circular paper substrate comprise a different substance specific for a different analyte. In another example, each row of a grid of paper substrates can be specific for a particular analyte, and each column can be specific for different analytes. In this example, the number of paper substrates in the columns of the grid can be related to the number of different analytes that can be tested, and the number of paper substrates in the rows of the grid can be related to the error checking redundancy of the test.

The device may comprise at least one electrode. The at least one electrode may be at least one electrode as described elsewhere herein. The at least one electrode may be configured to detect the signal as described elsewhere herein. The at least one electrode may comprise two or more electrodes. The two or more electrodes may be configured in a concentric arrangement. For example, a first electrode can be surrounded by a second electrode. The concentric arrangement may be a target pattern. The two or more concentric electrodes may be configured to be sequentially charged to sequentially enrich for the one or more analytes. For example, for a device comprising three electrodes, the outermost electrode can be charged to enrich the area of the outermost electrode with analytes. In this example, the middle electrode can then be charged to concentrate the analytes into the smaller area of the middle electrode, and subsequently the center electrode can be charged to concentrate the analytes into the smaller area of the center electrode. In this example, the sequential charging of the concentric electrodes can increase the final concentration of analytes at the center electrode. The two or more electrodes may be configured in a grid, a series of lines, any geometric shape (e.g., a triangle, a square), or the like. For example, a plurality of electrodes can be configured as a hexagonal grid of electrodes. The at least one electrode may comprise a metal (e.g., gold, silver, copper, platinum), an alloy (e.g., brass), a semiconductor (e.g., silicon), carbon (e.g., graphite, glassy carbon, graphene), an organic conductor (e.g., a polymer electrode), or the like, or any combination thereof. The at least one electrode may comprise conductive carbon paper. For example, a paper substrate can be impregnated with graphite, thus generating a conductive paper electrode. In another example, a graphite sheet can be an electrode. In another example, a conductive carbon layer can be drawn onto the surface of a membrane with carbon sticks, painted with conductive carbon paint, screen printed with conductive carbon paste, inkjet printed with conductive ink, or the like, or any combination thereof.

FIG. 1 is an example of a biomolecule test module and associated dock. The module may comprise a housing 100. The housing 100 may be hollow to enable storage of one or more components of the biomolecule test. For example, a hollow housing may comprise reagent containers, a sample receptacle, and detection circuitry. The housing 100 may comprise, plastic (e.g., polyethylene, polystyrene, resin, etc.), metal (e.g., aluminum, iron, copper), fiber-based materials (e.g., paper, cardboard, etc.), or the like, or any combination thereof. The housing 100 may be opaque, translucent, or transparent. For example, the housing can be a clear plastic hollow tube. The housing may be rigid. The housing may be soft (e.g., the housing is deformable). For example, the housing can be a soft plastic such that a subject can manipulate elements within the housing. The housing 100 may have a barcode 101 placed on or in the housing. The barcode may identify the contents of the housing 100. For example, the barcode can provide information about what analyte the housing comprises reagents to test for. The barcode may be related to other information about the biomolecule test module such as, for example, lot number, expiration date, usage instructions, efficacy information, or the like, or any combination thereof. The information related to the barcode may be displayed to a subject subsequently to the subject scanning the barcode with a device. For example, a subject can take a picture of the barcode with the subject's smartphone, and the smartphone can then display the instructions of how to use the test module to the subject.

The housing 100 may comprise a transparent window 102. The window may be configured to display a detection chamber located within the housing 100. The detection chamber may be as described elsewhere herein (e.g., comprising colorimetric and/or electronic detection). For example, a colorimetric test area can be visible to a user via window 102 for the user to image the test area with a smartphone. The window 102 may comprise the same material as the housing 100, or the window may comprise one or more different materials. For example, a cardboard housing 100 can have a polyethylene window to permit viewing of the detection chamber. Surrounding the window can be calibration color strips 103. The calibration color strips may be color strips as described in FIG. 17.

The housing 100 may comprise a sample port capped by sample port cap 104. The sample port may permit loading of the sample into the test module. For example, a user can place a swab into the sample port and seal the port with cap 104. The cap 104 may be impermeable (e.g., gas and liquid tight). The cap 104 may be permeable (e.g., gas permeable). For example, the cap can be gas permeable permit gas flow to allow the sample to enter the test module, but liquid impermeable to prevent the sample from spilling. The cap 104 may comprise one or more feedback mechanisms such as, for example, those described in FIGS. 79-81. The biomolecule test module comprising housing 100 may be configured to interface with test stand 105. The test stand may be portable as described elsewhere herein. The test stand 105 may comprise one or more power sources (e.g., one or more batteries, capacitors, photovoltaic panels, engines, generators, plugs to wall power, etc.). For example, the test stand can be a battery powered test stand. The test stand 105 may comprise one or more processors. The one or more processors may be configured to control elements of the biomolecule test module, such as, for example, reagent flow, heating temperature, reaction time, product flow, analyte concentration, other aspects as described elsewhere herein, or the like, or any combination thereof. For example, the test stand can comprise a processor configured to perform a nucleic acid amplification reaction within the test module and detect products of the amplification reaction using an electrode within the module. The test stand 105 may comprise one or more status indicators 106. The status indicators may be lights (e.g., light emitting diodes), speakers, displays, or the like, or any combination thereof. For example, the status indicators can be a red, yellow, and green light emitting diode to indicate the current status of the test being performed in the test module. The status indicators may be configured to provide an update to a subject using the testing module. For example, a speaker status indicator can beep to indicate the test has finished.

The housing 100 may interface with the stand 105 via one or more interfaces. The one or more interfaces may comprise conductive interfaces (e.g., conductive pads on the housing and the stand), inductive interfaces (e.g., inductive coils within the housing and the stand), or the like, or any combination thereof. The housing 100 may not comprise any processors. For example, all processing for performing the test can be done by processors in the stand. The housing may be configured to be disposable. For example, the housing may be configured to be used once before being discarded. The stand may be configured to be reusable. For example, a plurality of housings can be inserted into a single stand over time and be used for testing.

FIG. 2 is an example of a biomolecule test module and associated circular dock. The biomolecule test module may be as described elsewhere herein, such as, for example, in FIG. 1. The round test stand 107 may show a different embodiment of test stand 105. The round test stand may have a smaller footprint than a different shaped test stand. The test stand may be round, square, rectangular, trigonal, or any other shape. For example, the test stand can be a hexagon. The test stand may comprise decorative aspects. For example, the test stand can be configured to be aesthetically pleasing.

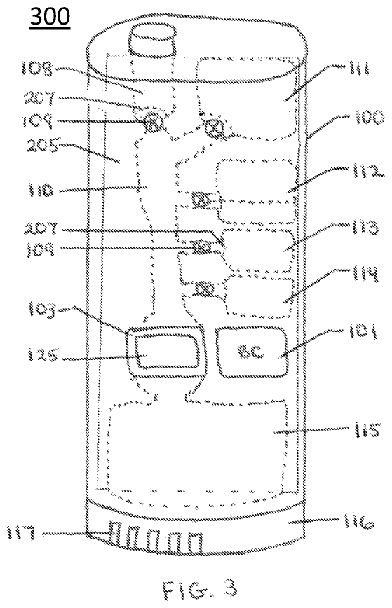

FIG. 3 is an example of a biomolecule test module 300. The biomolecule test module may be configured to interface with a test stand such as, for example, test stands 105 or 107. The biomolecule test module may comprise a housing 100. The housing 100 may be configured to contain and protect the other elements of the biomolecule test module. Dashed lines may indicate elements contained within the housing 100. For example, barcode element 101 can be placed on the outside of the housing. The test module 300 may comprise a sample reservoir 108. The sample reservoir 108 may be configured to accept a sample from a subject. For example, a subject can spit into the reservoir. In another example, the subject can place a swab into the reservoir. The sample reservoir may comprise one or more reagents. The one or more reagents may be sample processing reagents. The sample processing reagents may comprise reagents for performing cell lysis (e.g., surfactants, bases, alcohols, etc.), reagents for fragmenting cellular materials (e.g., proteins, surfactants, etc.), solvents (e.g., water, polar solvents, non-polar solvents, etc.), or the like, or any combination thereof. The sample reservoir may not comprise any reagents. For example, the sample reservoir can contain the sample as provided from the subject. The sample reservoir may be a soft sample reservoir (e.g., a deformable reservoir). The soft sample reservoir may be configured to have pressure applied to the reservoir to expel the sample from the reservoir. For example, a subject can squeeze the reservoir to push the sample out of the reservoir. The sample reservoir may comprise a membrane seal 207. Membrane seal 207 may be a single membrane, a double membrane, a triple membrane, or the like. Membrane seal 207 may be a plastic membrane seal. The membrane seal may be configured to be ruptured upon application of pressure through the housing 100 to the sample reservoir 108. For example, a subject can squeeze the sample reservoir to burst the membrane seal.

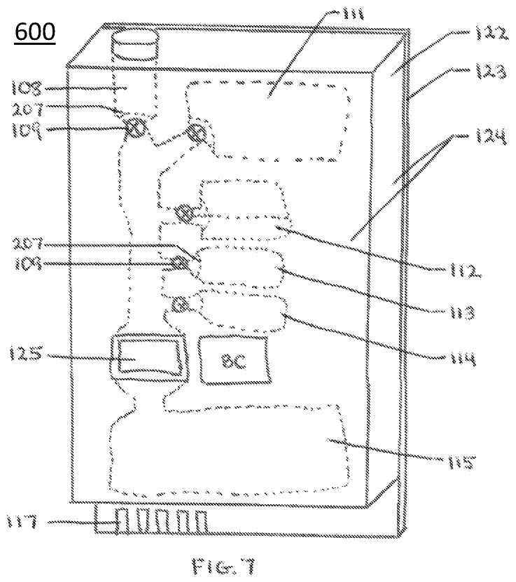

The sample reservoir 108 may be separated from fluidic channel 110 by a safety valve 109. The safety valve may be a valve as described elsewhere herein such as, for example, in FIGS. 10-14. The presence of the safety valve may decrease the likelihood of accidental rupture and flow of the reagents during transit of the test. For example, the safety valve can be strong enough that movement during shipping does not break it, but weak enough that a subject can break while performing a test. The safety valve may be configured to prevent the sample from moving from the sample reservoir to the fluidic channel without activation by a subject. For example, membrane 207 can have broken in transit, but the safety valve can prevent the flow of the sample before the subject is ready. Each reservoir of the test module may be adjacent to a membrane seal such as membrane seal 207 and a safety valve such as safety valve 109. The fluidic channel may not be a microfluidic channel. The fluidic channel may have a capacity of at least about 0.01, 0.05, 0.1, 0.2, 0.3, 0.4, 0.5, 0.6, 0.7, 0.8, 0.9, 1.0, 1.1, 1.2, 1.3, 1.4, 1.5, 2.0, 2.5, 5.0, 7.5, 10.0, or more milliliters. The fluidic channel may have a capacity of at most about 10.0, 7.5, 5.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.05, 0.01, or less milliliters. The fluidic channel may be a soft fluidic channel. For example, a subject can squeeze a sample along the length of the fluidic channel to push the sample along the fluidic channel. The fluidic channel may comprise one or more membrane-based substrates. For example, the fluidic channel can comprise a membrane-based substrate configured to direct a sample based on wetting forces. The fluidic channel may be configured to direct a sample from the sample reservoir to the reaction and/or detection chamber 125. For example, a user can burst safety valve 109 and push a sample down fluidic channel 110 into reaction and/or detection chamber 125. The fluidic channel may be insensitive to a presence of gas within the fluidic channel. For example, the fluidic channel can have air in the fluidic channel without disrupting the transport of liquids through the fluidic channel. The fluidic channel may comprise at least about 1, 5, 10, 20, 30, 40, 50, 60, 70, 80, 90, 95, 99, or more percent gas. The fluidic channel may comprise at most about 99, 95, 90, 80, 70, 60, 50, 40, 30, 20, 10, 5, 1, or less percent gas. The sample may be at a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The sample may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The sample may have a pH as defined by any two of the proceeding values. For example, the sample may have a pH from about 4.0 to about 6.0.

The test module 300 may comprise a rinse reservoir 111. The rinse reservoir may be a soft reservoir as described elsewhere herein. The rinse reservoir may comprise one or more rinse reagents. The one or more rinse reagents may be one or more solvents (e.g., water, alcohols, ethers, esters, etc.), one or more buffers, one or more surfactants, or the like, or any combination thereof. The one or more rinse reagents may be configured to rinse portions of the sample out of reaction and/or detection chamber 125. For example, the rinse reagents can rinse out unbound nucleic acids from the sample from the reaction and/or detection chamber. In addition to rinse reservoir 111, The test module 300 may comprise one or more additional rinse reservoirs, such as rinse reservoir 114. Each rinse reservoir may comprise the same rinse reagents. Each rinse reservoir may comprise different rinse reagents.

The test module 300 may comprise amplification reservoir 112. The amplification reservoir may be a soft reservoir as described elsewhere herein. The amplification reservoir may comprise one or more amplification reagents. The amplification reagents may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The amplification reagents may be loop mediated isothermal amplification reagents. The amplification reagents may be recombinase polymerase amplification (RPA) amplification reagents. The amplification reagents may be helicase dependent isothermal amplification reagents. The amplification reservoir may be activated subsequently to rinse reservoir 111. For example, a sample in reaction and/or detection chamber 125 can be rinsed with reagents from reservoir 111 and subsequently exposed to amplification reagents from reservoir 112. In this example, after an amplification reaction, the products can be rinsed with rinse reagents from reservoir 114.

The test module 300 may comprise a waste reservoir 115. The waste reservoir may be a soft reservoir as described elsewhere herein. The waste reservoir may be a hard reservoir (e.g., not a deformable reservoir). The waste reservoir may be configured to accept fluids (e.g., sample, reagents, etc.) that overflow from reaction and/or detection chamber 125. For example, rinse reagents from reservoir 111 can flow through the reaction and/or detection chamber and flow into the waste reservoir. The test module 300 may comprise a stand 116. Stand 116 may be a rigid stand (e.g., able to maintain the shape of the stand). Stand 116 may be a plastic stand, a thick paper stand (e.g., a cardboard stand), or the like, or any combination thereof. The stand 116 may comprise electrode connections 117. Electrode connections 117 may be configured to couple with electrode connections on the stand. Electrode connections 117 may be connected to one or more electrodes, heating elements, lighting units, detectors, or the like, or any combination thereof. For example, the electrode connections can be connected to a pair of electrodes and a heater. The stand may comprise at least about 1, 2, 3, 4, 5, 6, 7, 8, 9, 10, 15, 20, or more electrode connections. The stand may comprise at most about 20, 15, 10, 9, 8, 7, 6, 5, 4, 3, 2, or fewer electrode connections.

The test module 300 may comprise a reaction and/or detection chamber 125. The chamber 125 may be a fluidic region as described elsewhere herein. The chamber 125 may comprise one or more membrane-based substrates as described elsewhere herein. The chamber 125 may be configured to contain one or more amplification reactions. The chamber 125 may be configured to contain one or more detections as described elsewhere herein. For example, the chamber can be adjacent to an electrode configured to perform an electrical detection. In another example, the chamber can have a transparent side configured to display a colorimetric signal for a colorimetric detection. In this example, the chamber may be imaged by a camera to detect the colorimetric signal. In another example, the chamber may be adjacent to a photodetector configured to detect a colorimetric signal. The test module 300 may comprise one or more solids chambers 202 configured for storage of solids as shown in FIG. 82. The solids may comprise dried reagents, lyophilized reagents, solid reagents, or the like, or any combination thereof. The reagents may be reagents as described elsewhere herein. The reagents may be contacted with liquids from reservoirs 201 upon activation of safety valves 109. The liquids from the reservoirs may dissolve and/or suspend the solids for flow through channel 110. The chambers 202 may be in a same test cartridge 600 as reservoirs 111-114. The addition of the solids chambers permits the use of solid reagents that may have a shorter shelf life in liquid form in addition to liquid reagents.

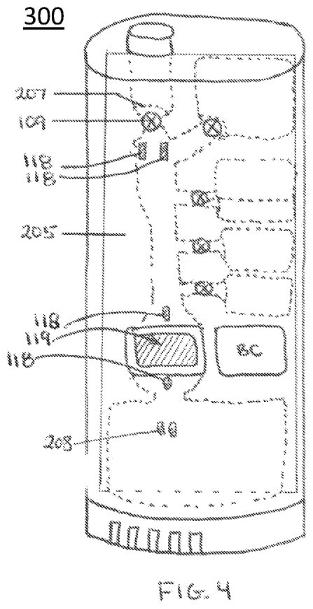

FIG. 4 is an example biomolecule test module 300 showing example internal electrode placement. The electrodes may be connected with electrode connections 117. The electrodes may be configured to be controlled by one or more processors within a stand. For example, a processor within a stand can send and receive signals to and from the electrodes via the electrode connections. The electrodes may comprise a metal (e.g., gold, silver, copper, platinum), an alloy (e.g., brass), a semiconductor (e.g., silicon), carbon (e.g., graphite, glassy carbon, graphene), an organic conductor (e.g., a polymer electrode), or the like, or any combination thereof. The electrodes may comprise conductive carbon paper. The electrodes may comprise carbon composite electrodes as described elsewhere herein.

Electrodes 118 of test module 300 may be configured as lysis electrodes, fluidic movement detection electrodes, fluid handing electrodes, or the like, or any combination thereof. The lysis electrodes may be configured to be biased in one or more pulses to aid in the degradation of cells and/or cellular bodies. For example, applying a series of pulses between the electrodes can aid in the lysis of cells from the sample, thus improving collection of analytes from the cells. The lysis electrodes may be biased with a voltage of at least about 0.1, 0.5, 1, 1.5, 2, 2.5, 3, 3.5, 4, 4.5, 5, 7.5, 10, 20, 30, 40, 50, or more volts. The lysis electrodes may be biased with a voltage of at most about 50, 40, 30, 20, 10, 7.5, 5, 4.5, 4, 3.5, 3, 2.5, 2, 1.5, 1, 0.5, 0.1, or less volts. The lysis electrodes may be biased for at least about 0.001, 0.005, 0.01, 0.05, 0.1. 0.5, 1, 1.5, 2, 3, 4, 5, or more seconds. The lysis electrodes may be biased for at most about 5, 4, 3, 2, 1.5, 1, 0.5, 0.1, 0.05, 0.01, 0.005, 0.001, or less seconds. The lysis electrodes may be biased at least about 1, 2, 3, 4, 5, 6, 7, 8, 9, 10, 25, 50, 75, 100, 250, 500, 750, 1,000, 1,250, 1,500, 1,750, 2,000, 2,500, 3,000, 4,000, 5,000, or more times in sequence. The lysis electrodes may be biased at most about 5,000, 4,000, 3,000, 2,500, 2,000, 1,750, 1,500, 1,250, 1,000, 750, 500, 250, 100, 75, 50, 25, 10, 9, 8, 7, 6, 5, 4, 3, 2, or less, times in sequence. For example, the lysis electrodes can be biased at 3 volts for 10 milliseconds 1,000 times to aid in cell lysis. The fluidic movement detection electrodes may be electrodes configured to detect a movement of a fluid past the electrodes. For example, a fluid flowing past a pair of electrodes 118 can complete a circuit between the electrodes, and the conductance of the circuit can indicate to a processor that fluid is flowing past the pair of electrodes. In another example, the presence of a conductive path between electrodes 118 adjacent to valve 109 and adjacent to chamber 125 can indicate presence of fluid along the length of the fluidic chamber. Fluid handling electrodes can be electrodes configured to move fluid at least in part due to the application of a potential across the electrodes. For example, applying a potential can change the wetting characteristics of a fluid and improve fluid flow past the electrodes. In another example, positively biased electrodes can attract a negatively charged fluid, thus effecting control of the fluid. Electrodes 118 may be optional. For example, the test module 300 may not comprise any freestanding electrodes.

Electrode 119 may be an electrode as described elsewhere herein (e.g., an electrode configured to concentrate, provide conditions for a reaction, and/or detect). The electrode may comprise a metal electrode (e.g., copper, silver, platinum). The electrode may comprise an alloy electrode (e.g., brass). The electrode may comprise a carbon electrode (e.g., a graphite electrode, a glassy carbon electrode, a graphene electrode). The electrode may comprise an electrode comprising a support (e.g., a paper support impregnated with graphene, a polymer support impregnated with graphite). The electrode may comprise a polymer electrode (e.g., a polypyrrole electrode, a poly(3,4-ethylenedioxythiophene) electrode). The electrode may comprise a semiconductor electrode (e.g., an organic semiconductor, an inorganic semiconductor). The electrode may comprise a combination of electrodes described elsewhere herein. For example, the electrode can be a graphite impregnated paper electrode connected to a copper electrode.

The test module 300 may comprise support 205. Support 205 may be a planar support (e.g., a solid sheet). The support may be configured to assist holder 100 in maintaining the shape of the test module. The support may be plastic, a metal, or the like, or any combination thereof. For example, the support can be a stiff sheet of plastic configured to hold up the test module. The support may be coupled to the reservoirs, electrodes, etc. of the test module. For example, the reservoirs of the test module can be affixed to the support.

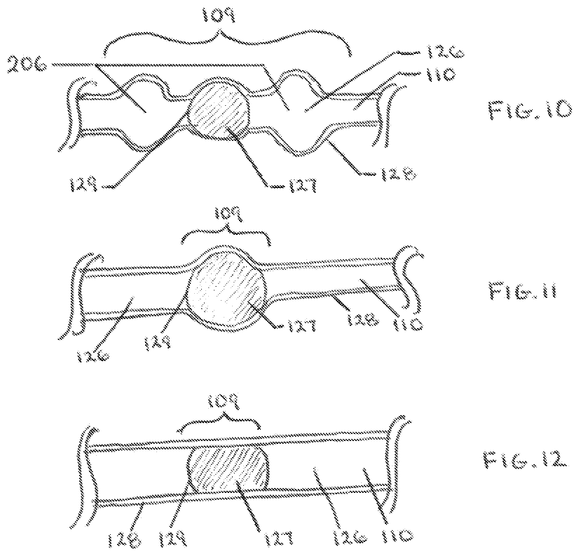

FIG. 5 is an example biomolecule test module comprising internal heating element 120. The internal heating element may be a heating element as described elsewhere herein such as, for example, in FIGS. 15-16. The heating element 120 may be configured to maintain a preconfigured temperature within reaction and/or detection chamber 125. For example, the heating element can maintain the chamber at a temperature sufficient to perform an isothermal amplification reaction within the chamber. The heating element may have a zig-zag design as shown in FIG. 5, or may have another configuration such as, for example, a spiral, a grid, a geometric shape, or the like. The heating element may be a resistive heating element (e.g., a resistor configured to heat upon flow of electricity through the resistor). The heating element may be an electrode as described elsewhere herein. The heating element may be connected to an electrical source (e.g., electrode connections 117) via connectors 121.

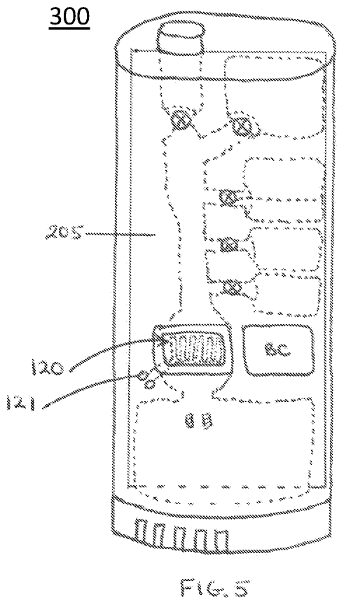

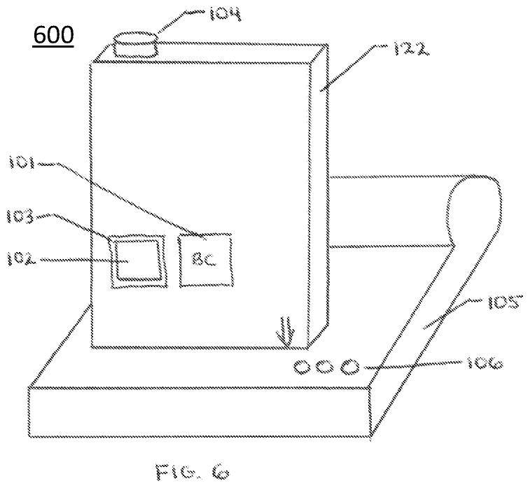

FIG. 6 is an example of a test cartridge 600 docked in a stand 105. Test cartridge 600 may comprise the same components as test module 300. For example, test cartridge 600 can comprise a barcode region 101 and calibration strips 103. FIGS. 7-9 show examples of possible internal components of test cartridge 600. Like markers may describe like elements with FIGS. 1-5. Test cartridge 600 may comprise a rigid backing 123. The rigid backing may be planar. The rigid backing may be one or more rigid backings. For example, a plurality of rigid rods can be a rigid backing. The rigid backing may be plastic, metal, cardboard, or the like, or any combination thereof. A casing 122 may be affixed to the rigid back 123. The casing 122 may be rigid. The casing 122 may be soft. For example, the casing may be a soft plastic membrane affixed to the rigid backing. The test cartridge 600 may comprise membrane 124. Membrane 124 may be a soft membrane. For example, interior elements of test cartridge 600 may be actuatable through membrane 124. Membrane 124 may be configured to contain a leak of any of the internal elements of test cartridge 600. For example, a leak from the rinse reservoir 111 can be contained within the cartridge 600 by membrane 124. Membrane 124 may be opaque, translucent, or transparent. Membrane 124 may surround the internal elements of test cartridge 600. For example, membrane 124 can comprise the border of reservoir 111 in FIG. 8. The test cartridge 600 may comprise one or more solids chambers 202 configured for storage of solids as shown in FIG. 82. The solids may comprise dried reagents, lyophilized reagents, solid reagents, or the like, or any combination thereof. The reagents may be reagents as described elsewhere herein. The reagents may be contacted with liquids from reservoirs 201 upon activation of safety valves 109. The liquids from the reservoirs may dissolve and/or suspend the solids for flow through channel 110. The chambers 202 may be in a same test cartridge 600 as reservoirs 111-114. The addition of the solids chambers permits the use of solid reagents that may have a shorter shelf life in liquid form in addition to liquid reagents.

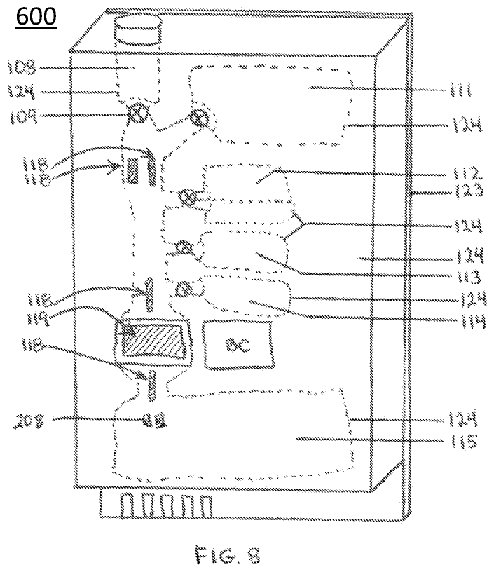

FIGS. 10-11 are example top-down views of a safety valve 109. FIG. 12 is an example of a side view of a safety valve 109. The safety valve may be a valve as described elsewhere herein. The valve may be configured to regulate a flow of fluid. For example, the valve can regulate a flow of fluid between a fluidic reservoir and a fluidic channel. The valve may comprise a chamber 127. The chamber may be pressurized (e.g., filled at a higher pressure than ambient pressure), unpressurized (e.g., filled at a same pressure as ambient pressure), or under pressurized (e.g., filled at a reduced pressure as compared to ambient pressure). The chamber may be filled with a gas, a liquid, or a combination thereof. The gas may be air. The gas may be an inert gas (e.g., nitrogen, a noble gas, etc.). The liquid may be water. The liquid may be an organic liquid (e.g., an alcohol). The liquid may comprise one or more reagents as described elsewhere herein. The chamber may be filled with a gas, a liquid, and a solid. For example, a solid powder can be suspended in the liquid. The chamber may be surrounded by membrane 129. Membrane 129 may be at least about 1, 5, 10, 25, 50, 75, 100, 250, 500, 750, 1,000, 2,500, 5,000, 7,500, 10,000 or more micrometers thick. Membrane 129 may be at most about 10,000, 7,500, 5,000, 2,500, 1,000, 750, 500, 250, 100, 75, 50, 25, 10, 5, 1, or less micrometers thick. Membrane 129 may be a plastic membrane, a metal foil membrane, or the like. Membrane 129 may be thinner than wall 128. For example, wall 128 can be 3 millimeters thick and membrane 129 can be 500 micrometers thick. Wall 128 may be configured to be deformable. For example, wall 128 can be deformable by application of pressure by a user using their fingers. Wall 128 may be rigid. For example, the wall can be made of a material that does not deform when a user applies pressure to it.

Membrane 129 may be configured to be rupturable upon an application of pressure to chamber 127. For example, a subject can press their finger on chamber 127, pushing the contents of the chamber into the membrane and thus rupturing the membrane. The membrane 129 may be configured to rupture upon application of at least about 0.01, 0.05, 0.1, 0.2, 0.3, 0.4, 0.5, 0.6, 0.7, 0.8, 0.9, 1, 2, 3, 4, 5, 6, 7, 8, 9, 10, or more megapascals. The membrane 129 may be configured to rupture upon application of at most about 10, 9, 8, 7, 6, 5, 4, 3, 2, 1, 0.9, 0.8, 0.7, 0.6, 0.5, 0.4, 0.3, 0.2, 0.1, 0.05, 0.01, or less megapascals.

Region 126 may be filled with a gas, a liquid, a solid, or any combination thereof. For example, region 126 can be an air gap region. In another example, region 126 can contain reagents from a reagent reservoir. Region 126 may be adjacent to compression chambers 206. In some embodiments, the safety valve does not comprise compression chambers 206, such as, for example, the safety valve of FIG. 11. The compression chambers may be filled with a gas, a liquid, a solid, or any combination thereof. The compression chambers may be configured to be used to burst a portion of membrane 129 that does not burst upon actuation of chamber 127. For example, a user applies force to chamber 127 and only the side of membrane 129 adjacent to fluidic channel 110 bursts. In this example, the user can press in the compression chamber opposite the burst membrane side to fully burst the membrane and permit fluid flow through the membrane space.

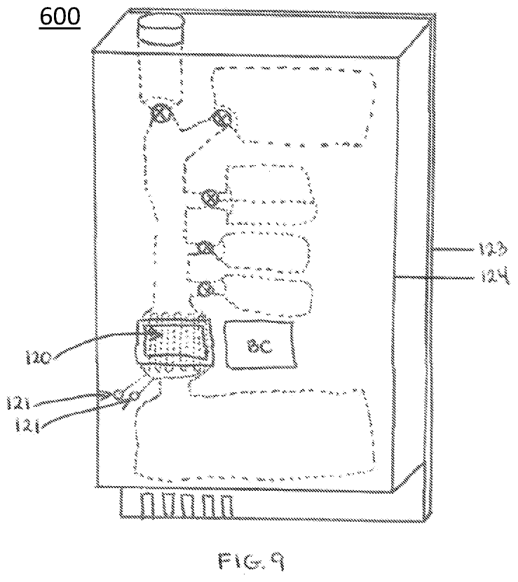

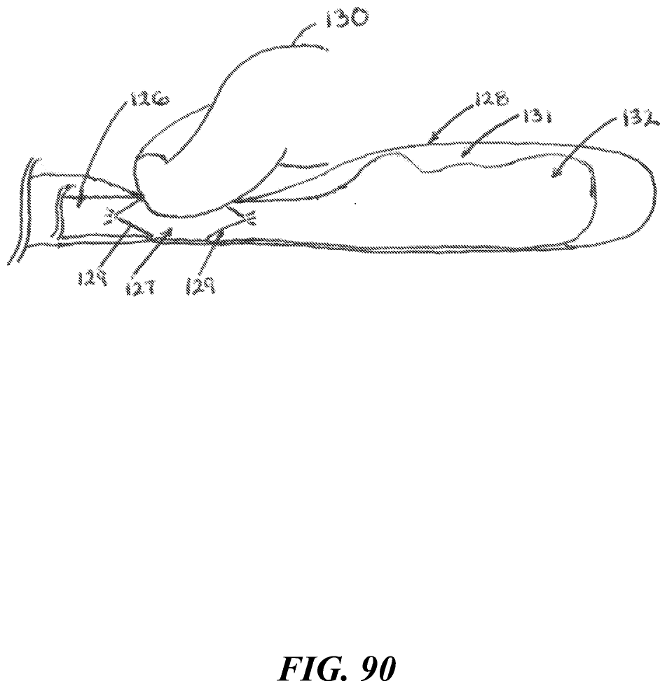

FIGS. 13-14 are an example diagram of actuating a safety valve 109 with a digit 130. The digit may be a thumb or finer of a subject. The digit may be a hard object (e.g., a rod, a pencil). Safety valve 109 may prevent a flow of fluid from reservoir 132 to region 126. The reservoir may comprise gas. For example, the reservoir can contain an air bubble. The subject may press the digit onto the valve 109, thereby rupturing the membrane of the valve and generating membrane fragments 133. Membrane fragments 133 may not impede a flow of a fluid from reservoir 132 through region 126. Once the membrane of the valve has been ruptured, the user can place the digit over reservoir 132 and press through wall 128 and flexible wall 131 to pressurize the material within the reservoir and burst membrane 207, thus permitting fluid flow from the reservoir through region 126. The reservoir may be a reagent reservoir, a rinse reservoir, or the like. In some embodiments, the reservoir may not comprise burst membrane 207 (e.g., FIG. 90). For example, the reservoir can be in direct fluidic and/or gaseous communication with the safety valve. In this example, the safety valve is sufficient to prevent the flow of the contents of the reservoir into the region.

FIGS. 15-16 are examples of heating element 120. In some embodiments, the heating element 120 can have a snaking pattern (e.g., the pattern shown in FIG. 15), a geometric pattern (e.g., a square wave, a sawtooth pattern), or the like. In some embodiments the heating element 120 is a single shape. The single shape may be a rectangle (e.g., the heating element of FIG. 16), another geometric shape (e.g., a triangle, an octagon), an arbitrary shape (e.g., configured to be the same shape as an arbitrary substrate), or the like. The heating element 120 may be connected to other circuitry via connectors 121. For example, the heating element can be connected to control or monitoring circuitry via the connectors. The heating element may be placed on a non-conductive pad 134. The non-conductive pad may not be electrically conductive, thermally conductive, or a combination thereof. The non-conductive pad may be a polymer pad (e.g., a nylon pad, a temperature resistant plastic pad), a fibrous material pad (e.g., a piece of cardboard), or the like. The non-conductive pad may comprise an adhesive. For example, the non-conductive pad can be a piece of tape. The heating element may be configured to have an operable range of temperatures of at least about 0, 5, 10, 15, 20, 21, 22, 23, 24, 25, 26, 27, 28, 29, 30, 31, 32, 33, 34, 35, 36, 37, 38, 39, 40, 45, 50, 55, 60, 65, 70, 75, or more degrees Celsius. The heating element may be configured to have an operable range of temperatures of at most about 75, 70, 65, 60, 55, 50, 45, 40, 39, 38, 37, 36, 35, 34, 33, 32, 31, 30, 29, 28, 27, 26, 25, 24, 23, 22, 21, 20, 15, 10, 5, 0, or less degrees Celsius. The heating element may be configured to have an operable range of temperatures as defined by any two proceeding values.

FIG. 17 is an example of a calibration strip 103. The calibration strip may comprise one or more fields 135. The fields may be black, white, one or more greyscale colors, a color of a positive result, a color of a negative result, primary colors, secondary colors, one or more of the ColorChecker® calibration colors, or the like, or any combination thereof. For example, a calibration strip can comprise a black panel, a white panel, a 50% grey panel, the color of a positive result, and the color of a negative result. The calibration strip may be configured to be detectable by a detector used for detecting a colorimetric signal. The calibration strip may improve the accuracy, specificity, and/or sensitivity of a colorimetric test by enabling color correction of the results of the test regardless of the local lighting of the test. For example, inconsistent lighting color temperature can shift the apparent color of colorimetric test, possibly reducing the accuracy. In this example, the calibration strip can be used to correct for the lighting of the test, and thus provide a more consistent test.

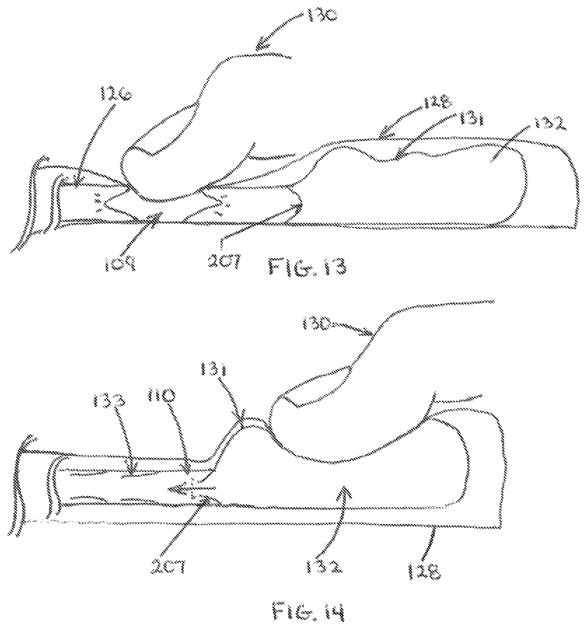

FIG. 18 is an example of a sample collector 136. The sample collector may be used with methods, devices, and tests described elsewhere herein. For example, the sample collector can be used to generate a sample comprising an analyte. The sample collector 136 may comprise a handle 138. The handle may be a plastic handle, a fiber-based handle (e.g., a wood handle), a metal handle, or the like, or any combination thereof. The handle may be connected to head 139 via breakable connection 137. Breakable connection 137 may be configured to be broken, thus separating head 139 from handle 138. The connection may be scored (e.g., have a scoring line across the handle), perforated (e.g., perforated with small holes), thinned (e.g., made thinner than the handle or the head), made of a weaker material (e.g., made of a more brittle plastic, made of plastic while the handle is made of metal), or the like, or any combination thereof. The breakable connection may be configured to break upon an application of at least about 1, 2, 3, 4, 5, 6, 7, 8, 9, 10, 50, 100, 500, 1,000 or more megapascals of pressure. The breakable connection may be configured to break upon application of at least about 1,000, 500, 100, 50, 10, 9, 8, 7, 6, 5, 3, 2, 1, or less megapascals of pressure. The breakable connection may be configured to be broken when the head 139 is placed into sample reservoir 108. For example, the head can be placed into the sample reservoir and broken off of the handle, permitting the sample reservoir cap to be placed on the reservoir. The head 139 may comprise one or more bristles 140. The bristles may be polymer bristles. The bristles may be configured for use in a manner similar to a toothbrush. For example, a subject can brush the sample collector around their mouth to collect saliva and cells from their mouth. The head may comprise one or more absorbent swabs (e.g., cotton swabs, polymer fiber swabs). The absorbent swabs may be placed under bristles 140 (e.g., the bristles protrude from the swab) or on the opposite side of head 139 from the bristles. The absorbent swabs may be configured to absorb fluids from the subject. For example, the absorbent swabs can absorb saliva from a subject's mouth.

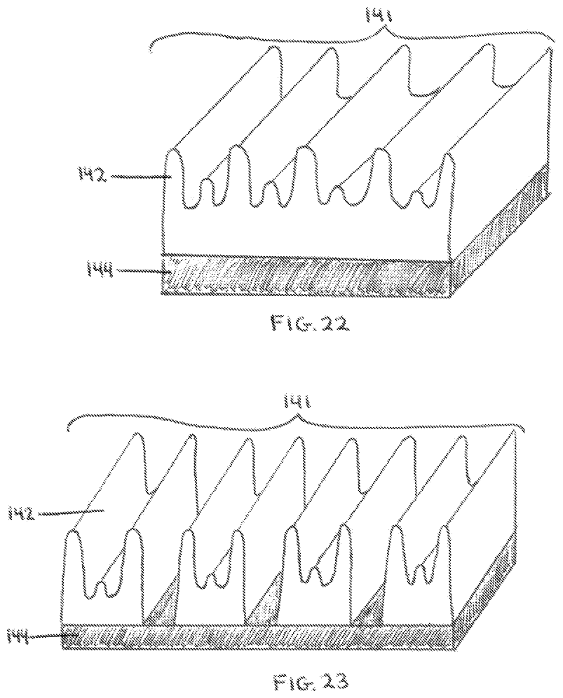

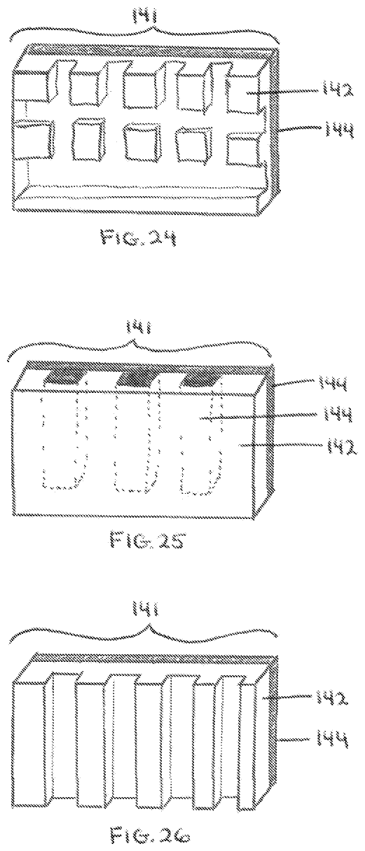

FIG. 19 is an example of a patterned composite electrode 141. The patterned composite electrode may comprise an electrode portion 144. The electrode portion may comprise a fiber-based electrode (e.g., a graphite impregnated paper-based electrode), a polymer-based electrode (e.g., a graphite treated nylon-based electrode), a metal electrode, or the like, or any combination thereof. The electrode portion may be a functionalized electrode. The functionalization may be an amine functionalization, a thiol functionalization, an acid functionalization (e.g., a carboxylic acid, a mineral acid), a halogen functionalization, a click chemistry functionalization (e.g., an azide, an alkyne), a nucleic acid functionalization (e.g., a nucleotide, an oligomer, a ribose nucleic acid, a deoxyribose nucleic acid), a protein functionalization (e.g., an antibody), an antigen functionalization, or the like, or any combination thereof. The patterned composite electrode may be a three-dimensional (3D) patterned electrode. For example, the patterned electrode can comprise features such as valley 143. The patterned composite electrode 141 may comprise absorptive element 142. The absorptive element may be a membrane as described elsewhere herein (e.g., a polymer membrane). The absorptive element may be paper. The paper may be filter paper (e.g., Whatman® filter paper). The paper may be 3D patterned. The 3D pattern may be a channel (e.g., FIGS. 22-23, 26), a grid (e.g., FIG. 24), a well (e.g., FIGS. 27-29), an arbitrary shape, or the like, or any combination thereof. For example, the 3D pattern can be the channels of FIG. 19. The paper may not be 3D patterned (e.g., FIG. 30).

The functionalization may comprise functionalization with a second material. The second material may be a coating of the membrane (e.g., a conformal coating of fibers of the membrane). The second material may be a plurality of particles adhered to the membrane (e.g., a plurality of nanoparticles, a plurality of microparticles, etc.). The second material may be an oxide. Examples of oxides include, but are not limited to, silicon oxides (e.g., silicon dioxide), zinc oxide, titanium oxide, other metal oxides, other insulative oxides, other semiconductor oxides, or the like, or any combination thereof. The inclusion of the second material may improve the binding of an analyte to the membrane (e.g., improved capture, improved retention during a wash process, etc.), improve a de-binding property of the membrane (e.g., improve a consistency of a removal of an analyte from the membrane during a recovery operation, etc.), or the like, or any combination thereof. For example, inclusion of silicon oxides on the surface of the membrane can improve both binding and washing properties of the membrane. The membrane functionalized with the second material can maintain a property of the membrane without the functionalization (e.g., flexibility, electrical conductivity/insulation, mechanical strength, etc.). For example, functionalization of a nylon membrane with silica nanoparticles can maintain a flexible membrane while imparting improved performance associated with the silica nanoparticles. The functionalization with the second material may comprise a functionalization of the membrane (e.g., a functionalization throughout the membrane, a functionalization of a surface of the membrane, etc.), a functionalization of the electrode (e.g., a functionalization of a surface and/or throughout a carbon electrode on a surface of the membrane), or a combination thereof. The second material may be functionalized as described elsewhere herein. For example, the second material can be functionalized with one or more of oligonucleotides, antimers, or antibodies. The composite electrode can be functionalized by both a first and a second material. For example, a portion of the composite electrode can have a silicon dioxide nanoparticle coating while a second portion of the composite electrode can have an oligonucleotide functionalization. In this example, the silicon dioxide portion can provide a binding surface for a plurality of biomolecules while the oligonucleotide can be specific to a particular analyte.

A composite electrode may be an electrode portion coupled to an absorptive element where the electrode portion can be read, energized, or the like, individually. For example, two absorptive elements coupled to two electrode portions that are electrically connected and energized as one can be a single composite electrode. In another example, four independent electrode portions that share a same absorptive element can be four composite electrodes. In this example, the array of independently addressable composite electrodes on the common absorptive element can be an array of composite electrodes. A composite electrode may have an increased structural stability as compared to a non-electrode membrane. For example, the presence of the electrode on the back of the membrane can improve the rigidity and structural strength of the membrane.

The patterned composite electrode 141 may comprise regions 145 and 146. Regions 145 and 146 may comprise one or more deposited reagents. The one or more deposited reagents may be reagents for an amplification reaction, a washing process, a detection process, or the like, or any combination thereof. The reagents for the amplification reaction may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The reagents for the amplification reaction may be recombinase polymerase amplification (RPA) amplification reagents. The reagents for the amplification reaction may be helicase dependent isothermal amplification reagents. The reagents for the washing process may comprise organic solvents (e.g., alcohols, ethers, esters), water, salts, ionic species (e.g., salts), buffers, or the like, or any combination thereof. The reagents for the detection process may comprise a binding dye, a fluorescent dye, a labeled nucleic acid strand, or the like, or any combination thereof. The one or more deposited reagents may be chemical reagents. The one or more deposited reagents may be biomolecular reagents (e.g., capture probes, antigens, etc.). The one or more deposited reagents may be different reagents for each feature. For example, each valley 143 can comprise different reagents. The reagents may be one or more gasses (e.g., air, inert gas, etc.). The gasses may be used as drying reagents.

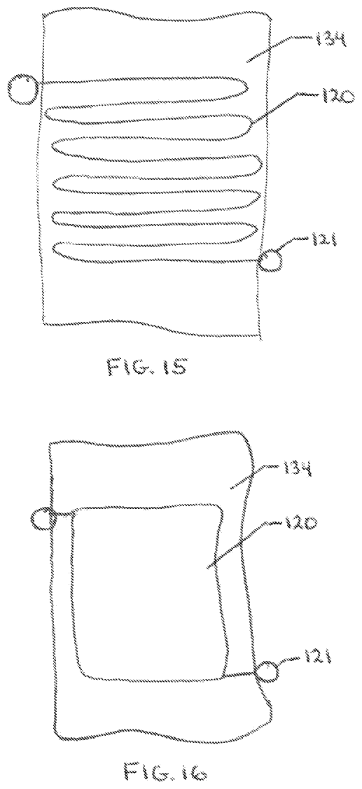

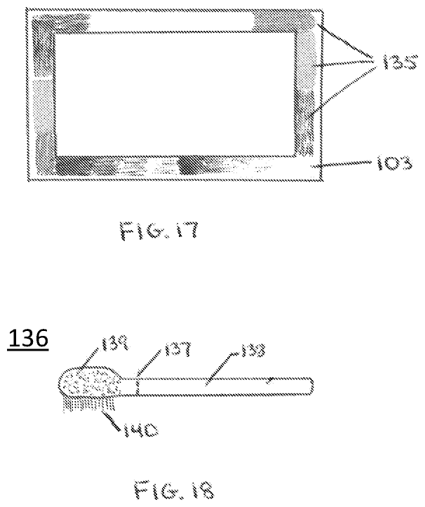

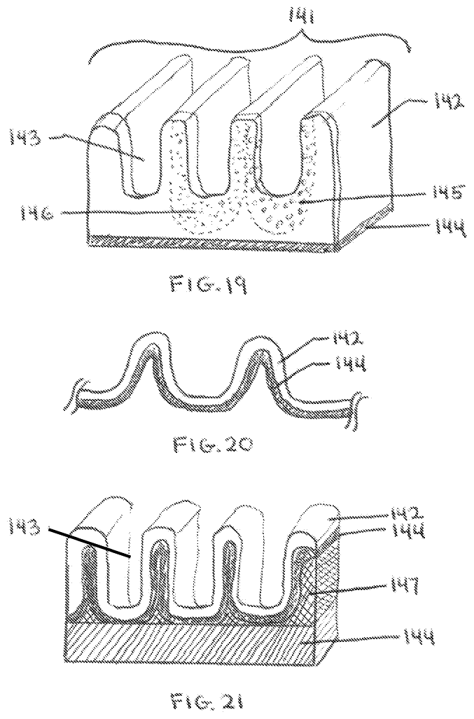

FIG. 20 is an example of a flexing composite electrode. The composite electrode may be configured to be deformable. For example, the composite electrode of FIG. 20 can be flexed to form the ridges shown in the figure. FIG. 21 is an example of patterned composite electrode. The patterned composite electrode may be configured to minimize a distance between the electrode 144 and the sample that can be adjacent to the channels. The electrode 144 may be connected from the conformal portion adjacent to absorptive element 142 to the planar portion by a conductive element 147. The conductive element may be a conductive polymer, a conductive adhesive, a metal, or the like, or any combination thereof. Positioning the electrode 144 within the valleys 143 can improve the ability of the electrodes to concentrate and/or detect analytes by decreasing the distance between the electrodes and the analytes. The conductive element may be configured to provide electrical attraction, electrical repulsion, holding (e.g., holding a charged or uncharged species in place), mixing (e.g., causing the mixing of two or more species), heating, or the like, or any combination thereof. The conductive element may be configured to be fully or partially in, on, or adjacent to the absorptive element. A portion of the conductive element not in contact with the absorptive element may be directly exposed to analytes, reagents, or the like, or any combination thereof. A portion of the conductive element not in contact with the absorptive element may be shielded with an inert covering. The inert covering may be a water impermeable covering. The inert covering may be an electrically non-conductive covering. The conductive element may comprise carbon (e.g., carbon black, glassy carbon, graphite, carbon fiber, drawn carbon pencil, graphene, graphite sheets, carbon paint, printed carbon, screen printed carbon, impregnated carbon, pressed carbon, etc.). The conductive element may comprise a metal (e.g., gold, platinum, copper, silver, etc.). The conductive element may comprise other conductive materials. A composite electrode may be configured to support capture, retention, modification, amplification, detection, or the like, or any combination thereof of analytes. Anywhere a composite electrode is found within the systems and methods of the present disclosure, additional other electrodes (e.g., counter electrodes, reference electrodes, etc.) may also be found. The additional other electrodes may comprise the same material as the composite electrodes. For example, a platinum counter electrode can be placed in electrical communication with a graphite impregnated nylon polymer composite electrode.

FIGS. 22-23 are examples of patterned composite electrodes 141. The multiple valleys of the patterning of absorptive element 142 shown in FIG. 22 can be configured to slow diffusion of reagents, analytes, analyte derivatives (e.g., amplification products), or the like, or any combination thereof from the surface of absorptive element 142. For example, a nucleic acid amplification reaction that occurs at the surface of the absorptive element can generate detectable analytes. In this example, the multiple valleys can slow the diffusion of the analytes away from the absorptive element, thus increasing the likelihood of the analytes being detected by a colorimetric detection reagent impregnated within the absorptive element. By separating the absorptive elements as shown in FIG. 23, a dispersion of reagents impregnated within the absorptive elements can be reduced. For example, if each absorptive element comprises a different oligonucleotide, separating the absorptive elements can slow diffusion between the absorptive elements, thus reducing cross contamination by the different reagents. The division of the absorptive elements can also reduce capillary transfer of reagents and/or analytes from one absorptive element to another. FIG. 25 is an example of a composite electrode 141 with a varying depth of absorptive element 142. Though the surface of absorptive element 142 may be flat, charged molecules may gather over the shallower portions of the element due to the closer proximity to the electrode 144. For example, for a positively charged electrode, negatively charged nucleic acid molecules can concentrate above the protruding portions of the electrode due to the distance dependence of the electric field.

FIGS. 27-29 are examples of patterned composite electrodes 141. A composite electrode 141 comprising absorptive element 142 may have a functionalized region 146. Region 146 may comprise one or more deposited reagents. The one or more deposited reagents may be reagents for an amplification reaction, a washing process, a detection process, or the like, or any combination thereof. The reagents for the amplification reaction may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The reagents for the amplification reaction may be recombinase polymerase amplification (RPA) amplification reagents. The reagents for the amplification reaction may be helicase dependent isothermal amplification reagents. The reagents for the washing process may comprise organic solvents (e.g., alcohols, ethers, esters), water, salts, ionic species (e.g., salts), buffers, or the like, or any combination thereof. The reagents for the washing process may comprise salt solutions in the reagents. For example, a reagent mixture of alcohol and salts can be provided as a wash reagent. In this example, the alcohols may be present in a range up to about 100% of the final volume of the reagents. The reagents for the detection process may comprise a binding dye, a fluorescent dye, a labeled nucleic acid strand, or the like, or any combination thereof. The one or more deposited reagents may be chemical reagents. The one or more deposited reagents may be biomolecular reagents (e.g., capture probes, antigens, etc.). The reagents may be one or more gasses (e.g., air, inert gas, etc.). The gasses may be used as drying reagents. The reagents may comprise one or more elution reagents. The elution reagents may be configured to de-bind the analyte from the membrane. The elution reagent may have a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The elution reagent may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The elution reagent may have a pH as defined by any two of the proceeding values. For example, the elution reagent may have a pH from about 7.5 to about 8.5. The elution reagent may comprise one or more buffers (e.g., tris(hydroxymethyl)aminomethane (TRIS), ethylenediaminetetraacetic acid (EDTA), etc.). For example, the elution reagent can comprise TRIS buffered water with EDTA.

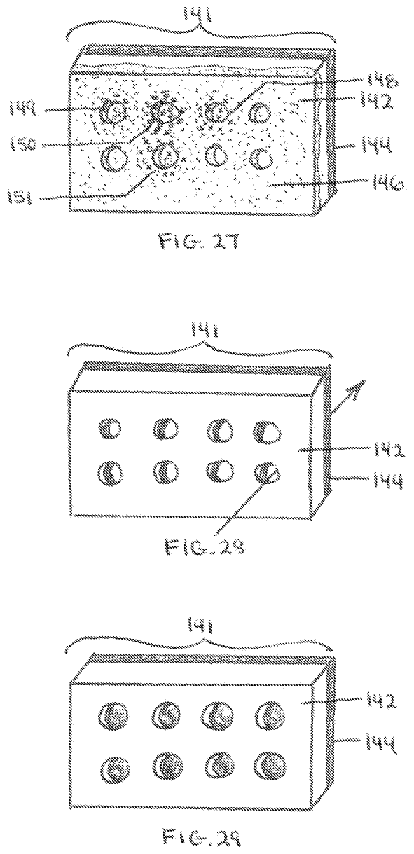

The absorptive element 142 may comprise additional regions 148, 149, 150, and 151. The additional regions may be functionalized in a same way as region 146. The additional regions may be functionalized differently from region 146. For example, region 146 can be functionalized with amplification reagents while regions 148, 149, 150, and 151 are functionalized with oligomeric primers. The additional regions may each be functionalized in a same way, a different way, or a combination thereof. For example, region 151 can be a combination of functionalizations of regions 149 and 148. The additional regions may be functionalization regions surrounding a feature of the absorptive element (e.g., a well). The additional regions may be separated (e.g., distinct from other regions). The additional regions may be overlapping (e.g., not distinct from other regions). The additional regions may be configured for detection of different analytes. For example, each region can be configured to generate signals for a different nucleic acid molecule. The separated additional regions may be configured to generate separated signals. For example, each region can generate a geographically separated and region-specific signal related to a presence or absence of an analyte. The absorptive element may comprise features (e.g., wells) having a bottom comprising the absorptive element (e.g., FIG. 27) or not comprising the absorptive element (e.g., having a through hole, FIG. 29). The composite electrode may comprise one or more holes through the absorptive element 142 and through the electrode 144 (e.g., FIG. 28).

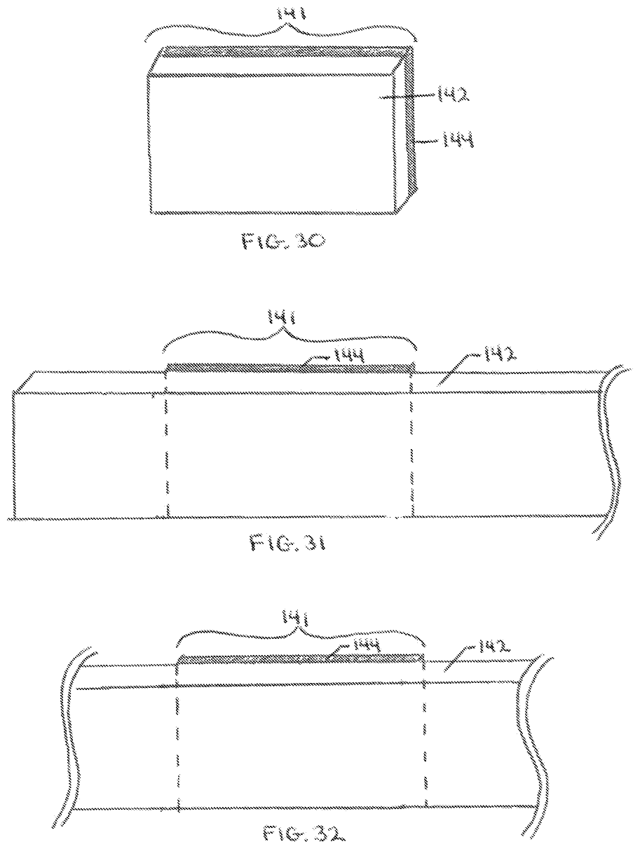

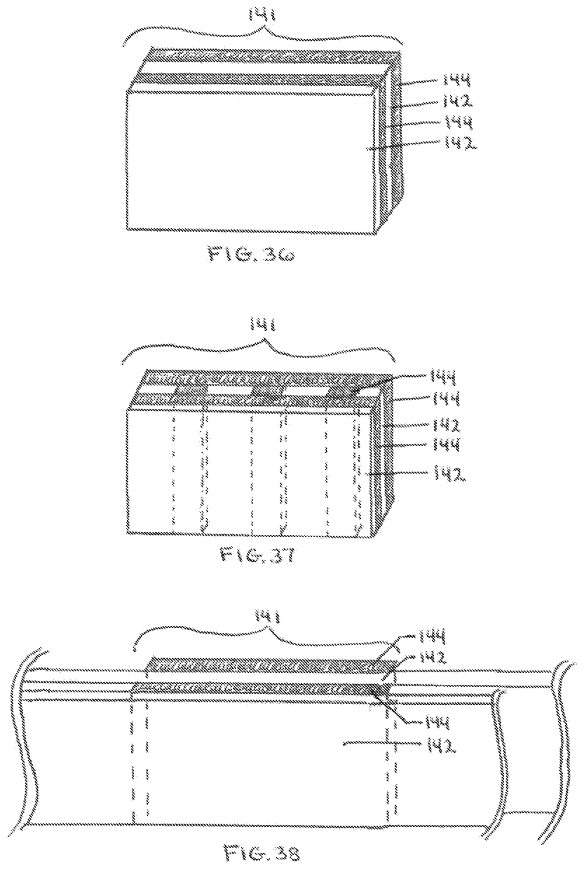

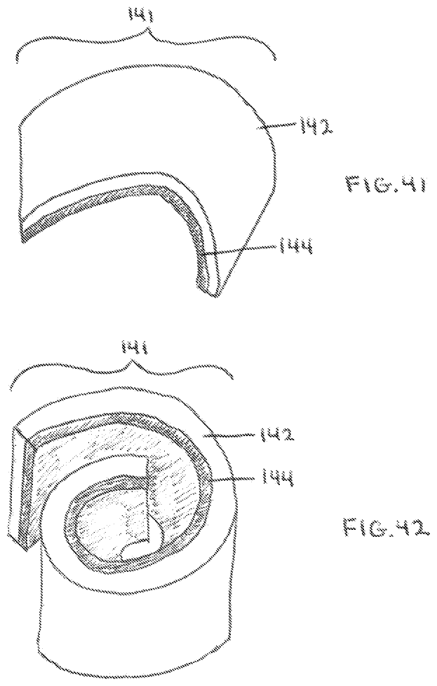

FIGS. 31-32 and FIG. 39 are examples of composite electrode 141. The absorptive element 142 may terminate near to the electrode 144 (e.g., FIG. 31). The absorptive element may terminate at a distance from the electrode 144 (e.g., FIGS. 32, 39). Extending the absorptive element beyond the bounds of the electrode can permit multiple regions across the absorptive element with different characteristics. For example, the absorptive element can be configured to flow a sample past the electrode, and a first electrode can be energized to concentrate analytes only in the region of the electrode. In this example, additional electrodes separated from the first electrode can be similarly energized, thus generating spatially separated concentrated regions of analytes. In another example, a first electrode can be positively charged and a second electrode can be negatively charged. In this example, negatively charged analytes can be concentrated at the region of the first electrode and positively charged analytes can be concentrated at the region of the second electrode. One or more electrodes 144 and absorptive elements 142 may be placed atop one another (e.g., FIG. 36). At least about 1, 2, 3, 4, 5, 6, 7, 8, 9, 10, or more electrodes and absorptive elements may be placed atop one another. At most about 10, 9, 8, 7, 6, 5, 4, 3, 2, or less electrodes and absorptive elements may be placed atop one another.

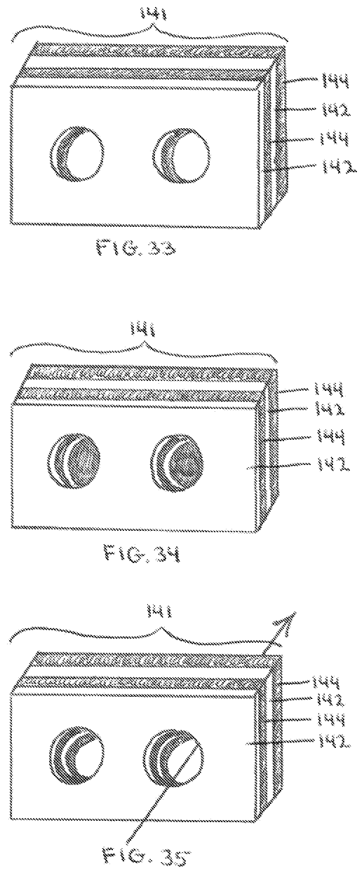

FIGS. 33-35 are examples of multi-layer composite electrodes 141. The multi-layer composite electrodes 141 may be one or more stacked electrodes 144. The electrodes may be electrodes as described elsewhere herein such as, for example, in FIGS. 27-29. The multi-layer composite electrodes may comprise features (e.g., wells) that extend through a first layer of the composite electrode (e.g., FIG. 33), that extend to a terminal electrode 144 of the composite electrode (e.g., FIG. 34), through the entire composite electrode (e.g., FIG. 35), or the like. Exposing inner electrodes of the composite electrode may result in stronger attraction of analytes and/or reagents to the electrodes. The stronger attraction can keep signals related to the presence or absence of the analytes well separated, resulting in improved properties of a test utilizing the composite electrodes. For example, a colorimetric test in which each well is configured to test for a different analyte, keeping the analytes and colorimetric signals spatially separated can reduce interference between the colorimetric signals. Depending on the size of the features, the composite electrode can be configured to operate as a sieve. For example, the composite electrode can trap charged species in trough holes of the electrode, removing the charged species from the solution flowing through the holes.

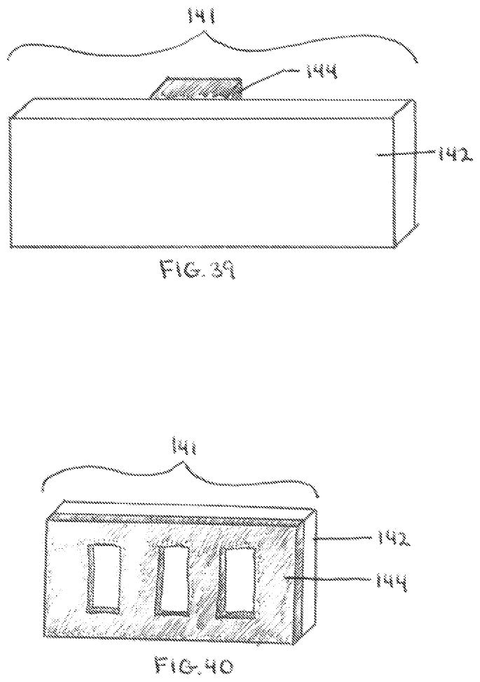

FIG. 37 is an example multi-layer composite electrode 141. The multi-layer composite electrode may comprise channels of absorptive material 142 between electrodes 144. The channels may be configured to influence and/or collect charged analytes as the analytes transit through the composite electrode. For example, analyte can be flowed through the channels of the absorptive material, and the electrodes along the channels can be charged to exert a force on the analyte. In this example, the electrodes can be configured to attract the analyte to concentrate near the surface of the electrode. FIG. 38 is an example multi-layer composite electrode 141 comprising electrodes 144 adjacent to extended absorptive elements 142. The multi-layer composite electrode 141 of FIG. 38 may be configured to concentrate analytes and/or reagents in the area of the electrodes 144. For example, a liquid sample comprising a charged analyte can be deposited on absorptive element 142 and adjacent to an electrode 144. In this example, as the sample flows into the region of the absorptive element between the electrodes, the electrodes can concentrate the charged analyte by application of a bias across the electrodes. In another example, reagents pre-loaded onto the absorptive element positioned between the electrodes can be activated by applying a bias across the electrodes configured to repel the pre-loaded reagents. In this example, the reagents can then flow out of the region between the electrodes for use in the adjacent absorptive element.

FIG. 40 is an example of a composite electrode 141. The composite electrode may comprise an electrode 144 comprising voids. The voids may be channels, wells, or the like, or any combination thereof. For example, the voids can be rectangular wells as shown in FIG. 40. The electrode 144 may be on the sample application side of the composite electrode. For example, the sample can be applied directly to the electrode. In another example, the sample can be flowed from a fluidic channel onto the electrode side of the composite electrode. The sample may flow through the voids and be absorbed by the absorptive element 142. The electrode 144 may be configured to attract charged analytes from within the sample to the portions of the absorptive element within the voids. For example, the electrodes can apply a variable bias to repel charged analytes, thus concentrating the analytes within the voids (e.g., because the voids are the furthest distance from the repulsive electrodes the charged analytes can travel).

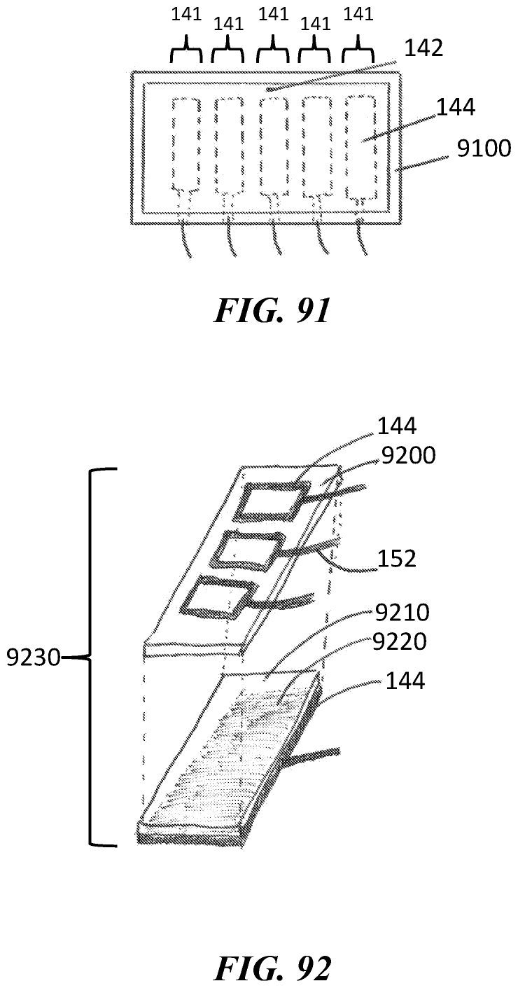

FIG. 91 is an example of a plurality of composite electrodes 141 comprising a same absorptive element 142. The plurality of composite electrodes 141 may comprise a plurality of electrodes 144 that are separate from one another. The regions of absorptive element 142 above each of the electrodes may be functionalized as described elsewhere herein. A plurality of the plurality of composite electrodes comprising a same absorptive element may be used in devices and methods described elsewhere herein. For example, two pluralities of composite electrodes can be placed in a reaction and/or detection chamber. The plurality of composite electrodes may comprise a protection element 9100. The protection layer may be a protection layer as described elsewhere herein.

FIG. 92 is an example of a stacked composite electrode 9230. The stacked composite electrode may comprise a top absorptive element 9200 and a bottom absorptive element 9210. The top and/or bottom absorptive elements may be absorptive elements as described elsewhere herein such as, for example, absorptive element 142. The top and bottom absorptive elements may be of a same material (e.g., both paper, both nylon membranes). The top and bottom absorptive elements may be of different materials (e.g., the top element is nylon and the bottom element is paper). The top absorptive element may comprise electrodes 144 electronically coupled to conductive traces 152. The electrodes may be patterned electrodes. The electrodes may be planar electrodes. The bottom absorptive element may comprise other electrodes 144. The other electrodes may be patterned electrodes. The other electrodes may be planar electrodes. For example, the top absorptive element can comprise printed electrodes while the bottom absorptive element can comprise a solid graphite electrode. The top electrodes and the bottom electrodes may be independently contacted, addressed, biased, etc. For example, the top electrodes can be negatively biased, and the bottom electrodes can be positively biased. In this example, a negatively charged analyte can be held near the bottom electrodes. In another example, one of the electrodes can be biased and the other electrode can be unbiased. The bottom absorptive element may comprise one or more reagents 9220. The one or more reagents may be one or more reagents as described elsewhere herein (e.g., probes, primers, detection reagents, etc.). The top absorptive element may comprise one or more reagents. The one or more reagents may be one or more reagents as described elsewhere herein (e.g., probes, primers, detection reagents, etc.). The top and bottom absorptive element may comprise the same reagents (e.g., the top and bottom elements can comprise amplification reagents). The top and bottom absorptive elements may comprise different reagents (e.g., the top element can comprise primers and the bottom element can comprise detection reagents).

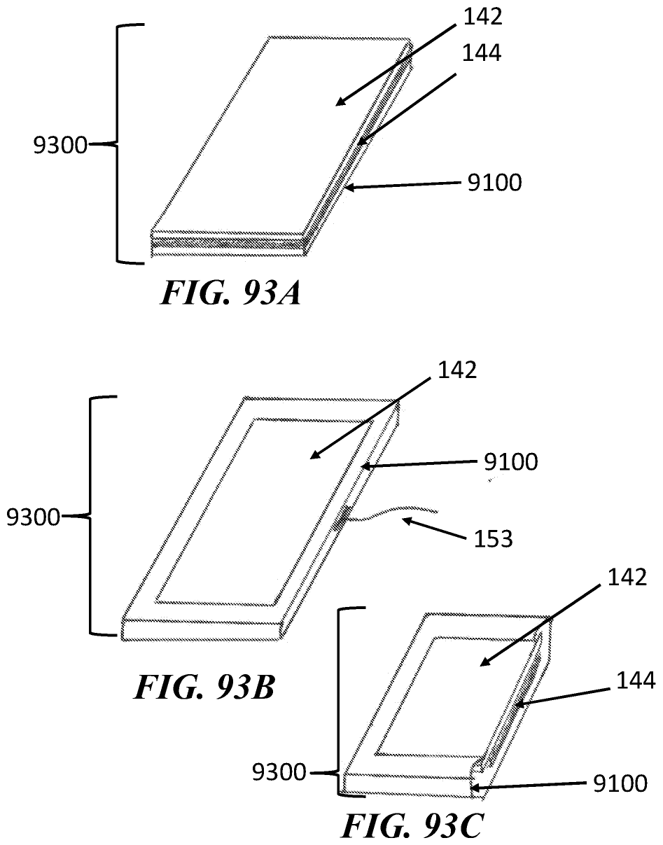

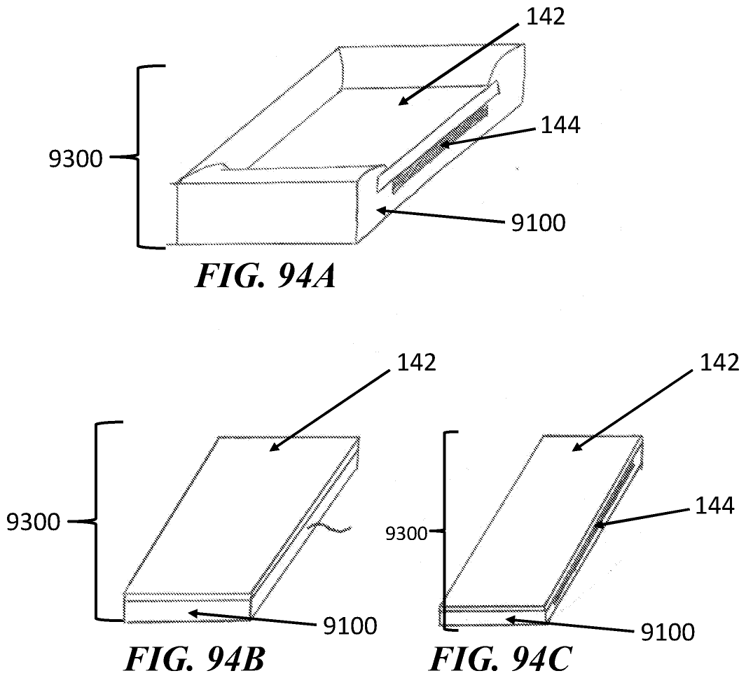

FIGS. 93A-93C and 94A-94C are examples of protected composite electrodes 9300. The protected composite electrode may comprise an absorptive element 142 and electrode 144 (e.g., similar to a composite electrode 141), as described elsewhere herein. The protected composite electrode may comprise protection element 9100. The protection element may be configured to be substantially electrically inert (e.g., not electrically conductive, resistant to degradation by electrical current and/or electric field, etc.). The protection element may be configured to be substantially impermeable to liquids (e.g., solvents, water, organic solvents, etc.), gasses (e.g., air, carbon dioxide, inert gasses, etc.), solutions (e.g., acidic solutions, basic solutions, buffered solutions, samples, etc.), and the like, or any combination thereof. The protection element may comprise a polymer protection element (e.g., polyethylene, plastic, nylon, etc.), a metal protection element (e.g., a metal foil, a metalized polymer, etc.), or the like, or any combination thereof. The protection element may comprise two or more protection elements. For example, a metal foil can serve as the impermeable portion of the protection element while a plastic film serves as the electrically inert portion. The protection element may comprise an adhesive. For example, a plastic tape can be the protection element with the adhesive side of the tape affixed to the electrode. The protective element may leave sides of the absorptive element and/or the electrode (e.g., FIG. 93A, FIGS. 94B-94C). The protective element may cover the sides of the absorptive element and/or the electrode, leaving the top of the absorptive element exposed (e.g., FIG. 93B). The protective element may comprise a port for a conductive lead 153 to provide electrical contact with the electrode. FIGS. 93C and 94A can be example cutaways showing the electrode sandwiched between the absorptive element and the protection element.

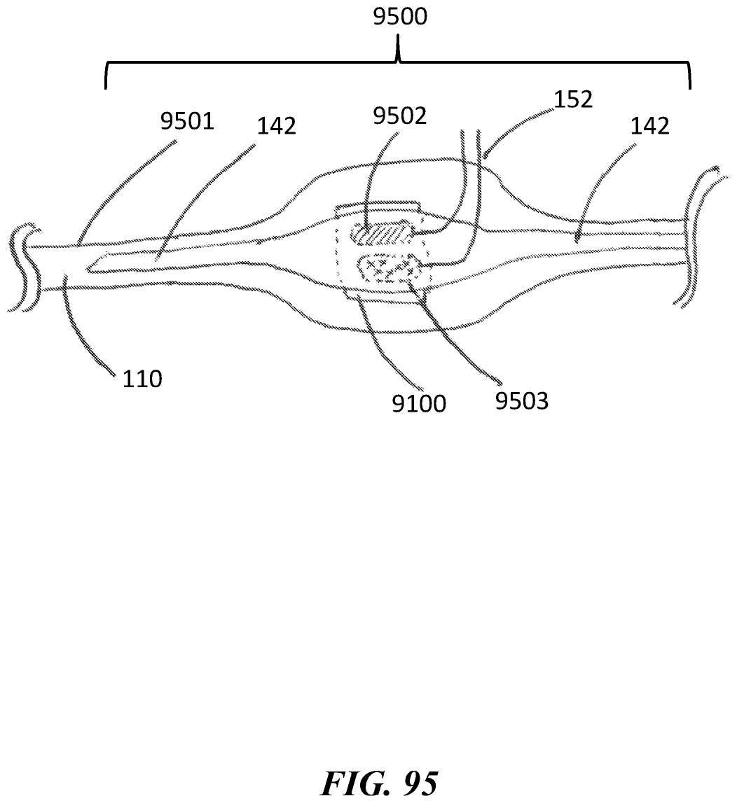

FIG. 95 is an example of an extended membrane composite electrode 9500. The composite electrode 9500 may be a composite electrode as described elsewhere herein. The composite electrode may be configured to be within a reaction and/or detection chamber surrounded by walls 9501 and in fluidic communication with channel 110. The composite electrode 9500 may comprise an extended absorptive element 142 that extends beyond electrodes 9502 and 9503. The electrodes may be electrodes as described elsewhere herein (e.g., electrode 144). The electrodes may be a single electrode. The electrodes may be a plurality of electrodes (e.g., a plurality of separate composite electrodes, a plurality of composite electrodes sharing an absorptive element, etc.). The extended absorptive element may be configured to guide a liquid (e.g., a sample, a liquid comprising one or more analytes) from the channel 110 towards the area of the absorptive element above the electrodes (e.g., by wetting forces). The extended portion of the absorptive element may not comprise 3D structuring. The portion of the absorptive element above the electrode may comprise 3D structuring as described elsewhere herein. For example, the absorptive element can be planar within the fluidic channel and have 3D ridges over the electrode. The whole absorptive element may be unstructured. The whole absorptive element may be structured. The electrodes may be adjacent to a protective element 9100 as described elsewhere herein. The region above the electrodes 9502 and 9503 may comprise one or more reagents as described elsewhere herein (e.g., amplification reagents, detection reagents, etc.). The regions of the absorptive electrodes above each of the electrodes may comprise different reagents. The regions of the absorptive electrodes above each of the electrodes may comprise same reagents. The extended membrane composite electrode may be adjacent to one or more waste reservoirs 115.

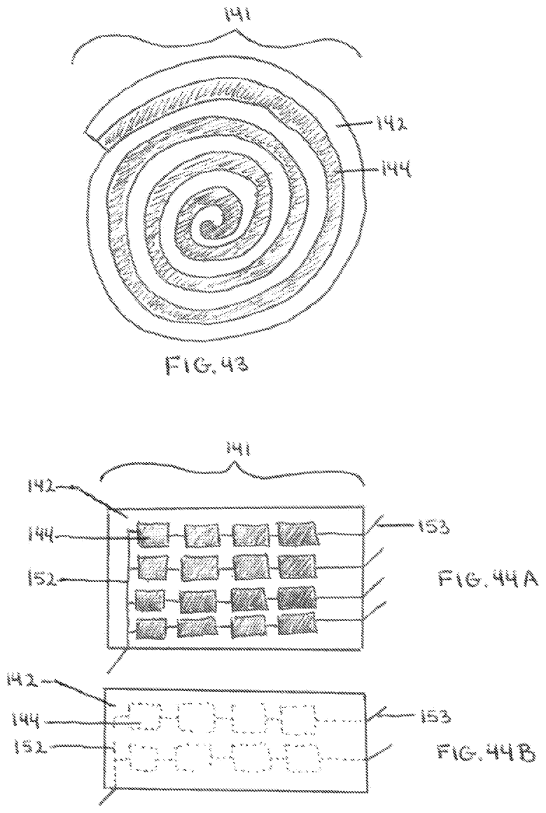

FIGS. 41-42 are examples of the flexibility of composite electrode 141. Flexibility may be the ability to flex at least about 1, 10, 50, 100, 150, 200, 250, 300, 360, 720, 1080, or more degrees. Flexibility may be the ability to flex at most about 1080, 720, 360, 300, 250, 200, 150, 100, 50, 10, 1, or less degrees. For example, a flexible composite electrode can flex 45 degrees. In another example, a flexible composite electrode can be rolled into a spiral shape such as, for example, the spiral of FIG. 42 or FIG. 43. Absorptive element 142 may be a flexible absorptive element. The flexible absorptive element may be a fiber-based absorptive element (e.g., paper, cotton), a polymer-based absorptive element (e.g., woven plastic), flexible variants of other materials described elsewhere herein (e.g., flexible nitro-cellulose, nylon, positively charged nylon, polytetrafluoroethylene, etc.). or the like, or any combination thereof. Electrode 144 may be a flexible electrode. The flexible electrode may be a fiber-based electrode (e.g., a paper impregnated with conductive materials (e.g., graphite)), a polymer-based electrode (e.g., a conductive polymer, a polymer impregnated with conductive materials), or the like, or any combination thereof.

FIG. 43 is an example of a spiral composite electrode 141. The spiral composite electrode may comprise flexible absorptive element 142 and flexible electrode 144. A spiral composite electrode may have a smaller footprint than a non-spiral composite electrode. For example, the spiral electrode can take up less room in a device than a planar composite electrode with the same area of absorptive element. The spiral composite electrode may be configured as a chromatography system. For example, a sample applied to the outside of the spiral electrode can wet the absorptive element and begin to propagate towards the center of the composite electrode. In this example, the electrode 144 can be configured to charge along the spiral from outside to inside, attracting charged analytes along towards the center of the composite electrode.

FIGS. 44A and 44B are examples of grid electrode patterning within the composite electrode 141. FIG. 44A may show the electrode side of the composite electrode, while FIG. 44B may show the absorptive elements side of the composite electrode. The composite electrode 141 may comprise at least about 1, 2, 3, 4, 5, 6, 7, 8, 9, 10, 25, 50, 75, 100, or more electrodes 144. The composite electrode 141 may comprise at most about 100, 75, 50, 20, 10, 9, 8, 7, 6, 5, 4, 3, 2, or less electrodes 144. The electrodes may be connected by conductive trace 152. The conductive trace may comprise carbon-based traces (e.g., graphite traces, glassy carbon traces, carbon paper traces, graphitized paper traces), metal traces (e.g., gold, silver, platinum, copper, etc.), semiconductor traces, or the like, or any combination thereof. The conductive traces may connect all of the electrodes 144. The conductive trace may connect a subset of the electrodes 144. For example, the conductive trace can connect a column of the electrodes, while a different conductive trace can connect a different column of electrodes. In this example, each column of electrodes can be individually addressable. Each subset of the electrodes may be addressed to apply a different bias and/or heat. For example, columns of electrodes can be sequentially activated to direct charged analytes from one side of the array to the other. Each electrode may be connected to its own conductive trace. For example, each electrode may be individually addressable. The conductive traces may be linked to conductive leads 153. The conductive leads may connect the electrodes to processors, monitoring equipment, or the like, or any combination thereof. The conductive leads may, for example, connect to electrode connection 117. Though shown in a square grid pattern, the electrodes 144 may be configured in a different configuration such as, for example, another geometric grid pattern (e.g., a hexagonal grid), a line, an amorphous configuration (e.g., a configuration without long range order), a configuration related to another part of a device (e.g., in the same shape as markings on the device), or the like, or any combination thereof.

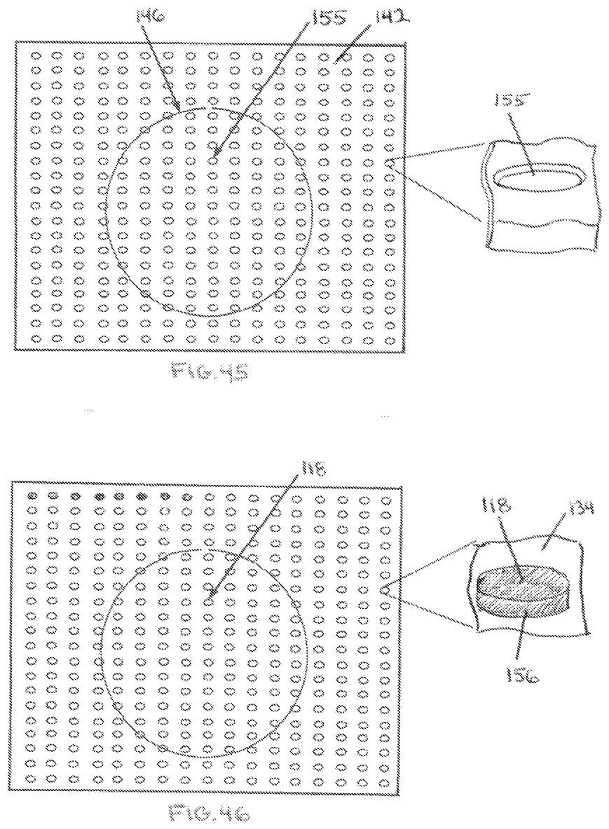

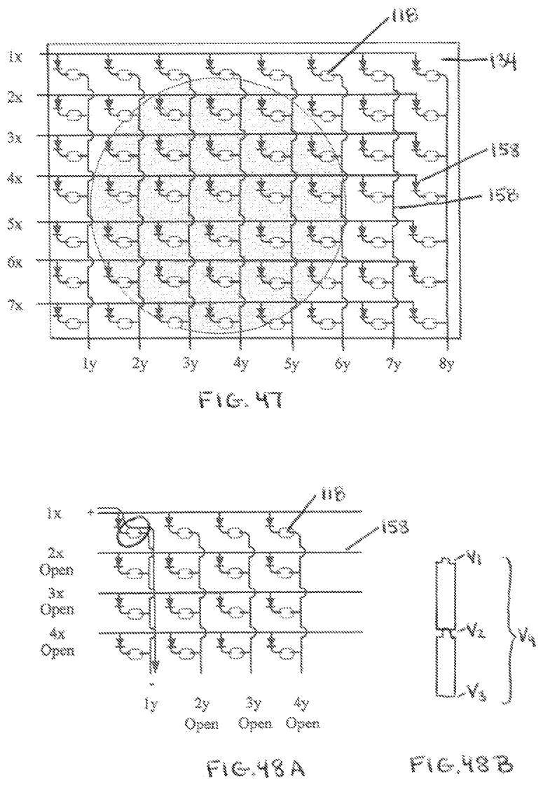

FIG. 45 is an example of an absorptive element 142 comprising a plurality of wells 155. Each well of the plurality of wells may be of the same dimensions (e.g., of the same width and depth). Wells of the plurality of wells may be of different dimensions. For example, one column of wells can be of a first width, and an adjacent column of wells can be of a second width. In another example, each well of the plurality of wells can be of different dimensions. The wells may have a dimension (e.g., height, width, depth, cross section, etc.) of at least about 1, 5, 10, 25, 50, 75, 100, 250, 500, 750, 1,000, 2,500, 5,000, 7,500, 10,000 or more micrometers. The wells may have a dimension of at most about 10,000, 7,500, 5,000, 2,500, 1,000, 750, 500, 250, 100, 75, 50, 25, 10, 5, 1, or less micrometers. The wells may have a geometric shape (e.g., circular, square, octagonal) or an arbitrary shape. The plurality of wells may be arranged in a square grid pattern as shown, or may be arranged in another geometric grid pattern (e.g., a hexagonal grid), a line, an amorphous configuration (e.g., a configuration without long range order), a configuration related to another part of a device (e.g., in the same shape as markings on the device), or the like, or any combination thereof. Wells of the plurality of wells may have a bottom comprising the same material as absorptive element 142. Wells of the plurality of wells may not have a bottom (e.g., be thru holes in the absorptive element).

The absorptive element may comprise region 146. Region 146 may comprise one or more deposited reagents. The one or more deposited reagents may be reagents for an amplification reaction, a washing process, a detection process, or the like, or any combination thereof. The reagents for the amplification reaction may comprise one or more polymerases, one or more salts, one or more buffers, one or more other enzymes (e.g., proteases), one or more organic solvents, one or more surfactants, one or more primers, one or more nucleotide triphosphates, loop mediated isothermal amplification reagents, or the like, or any combination thereof. The reagents for the amplification reaction may be recombinase polymerase amplification (RPA) amplification reagents. The reagents for the amplification reaction may be helicase dependent isothermal amplification reagents. The reagents for the washing process may comprise organic solvents (e.g., alcohols, ethers, esters), water, salts, ionic species (e.g., salts), buffers, or the like, or any combination thereof. The reagents for the detection process may comprise a binding dye, a fluorescent dye, a labeled nucleic acid strand, or the like, or any combination thereof. The one or more deposited reagents may be chemical reagents. The one or more deposited reagents may be biomolecular reagents (e.g., capture probes, antigens, etc.). The reagents may be one or more gasses (e.g., air, inert gas, etc.). The gasses may be used as drying reagents.

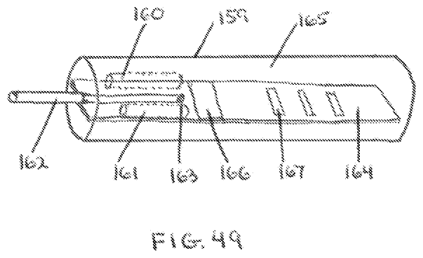

FIG. 46 is an example of a plurality of electrodes 118 configured to sit below the wells 155 of FIG. 45. The plurality of electrodes may be electrodes as described elsewhere herein. Each electrode may be individually addressable. Each electrode may be in electrical communication with an intersection of a row and column conductive matrix such as, for example, that shown in FIG. 47. For example, the electrodes can be in contact with the conductive matrix through holes in the non-conductive pad 134 at point 156. The electrodes may be configured to concentrate analytes and/or reagents within the wells 155 positioned above each electrode. FIG. 47 is an example of a diode matrix comprising diodes and traces 158. The diode matrix may be positioned on the other side of non-conductive pad 134 from the plurality of electrodes 118. The diode matrix may be a printable diode matrix (e.g., fabricated by printing). The diode matrix may comprise one or more multiplexers (e.g., semiconductor multiplexers), power transistors, microcontrollers, connections to other processors, connections to one or more power sources, or the like, or any combination thereof. In some embodiments, the diode matrix may be in electrical communication with one or more multiplexers (e.g., semiconductor multiplexers). The multiplexers may be housed within a stand. The multiplexers may be in electrical communication with one or more power transistors, microcontrollers, connections to other processors, connections to one or more power sources, or the like, or any combination thereof located within the stand. The diode matrix may be configured to individually control each of the plurality of electrodes 118. The control may comprise temporal control (e.g., the time of activation of the electrode), the bias applied (e.g., bias voltage, bias sign), or the like, or any combination thereof. For example, the diode matrix can be addressed by a processor operatively coupled to the diode matrix to sequentially bias each electrode of the plurality of electrodes with a different voltage. In another example, the diode matrix can be addressed by a processor to asynchronously bias each electrode of the plurality of electrodes (e.g., biased in any order).

The electrodes 118 may be in electrical communication with the traces 158 as shown in FIG. 48A. A particular electrode may be energized as shown by programming a controller (e.g., a microcontroller, a processor) to enable conduction at a specific row and column address. For example, the electrode at row 1, column 1 can be energized by applying the + and − signal as shown in FIG. 48A. FIG. 48 B is an example of a voltage available from a battery stack. For example, a processor can be configured to instruct application of a voltage (e.g., V1-V2) across a specific ROW and COLUMN address of the diode array and its associated carbon node. In this example a third potential (V3) can be applied, and referenced to V2, to a separate reference electrode in a flow channel, where upon application of the voltage the carbon node can become positively biased versus the reference electrode. In this example, the amount of current through the carbon node can be controlled such that the amount of heat generated at the node can be controlled. In this example, the application of a bias voltage, or heat if heat is predetermined to be applied, may be applied in a number of patterns to the matrix of composite electrodes, limited only by the number of electrodes and speed of switching specified by the controller. In this example, fast and precise control of the electrode matrix can be limited only by the speed of available electronics.

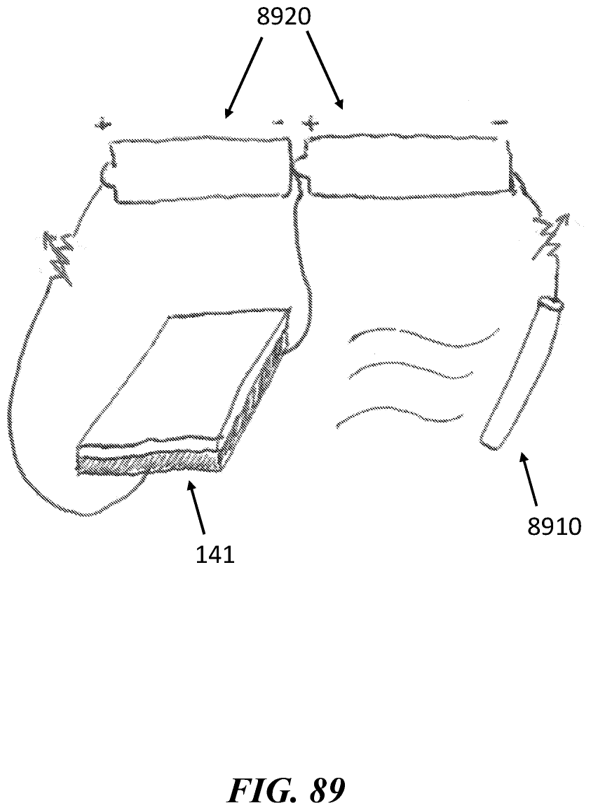

FIG. 89 is an example of a voltage application scheme. The voltage application scheme may result in a composite electrode 141 being held at a higher potential than a reference electrode 8910. Power sources 8920 may be one or more batteries, capacitors, connections to a power grid (e.g., wall socket), power supplies, or the like, or any combination thereof. In a case where a small current is passed through the composite electrode 141, the composite electrode-reference electrode circuit can operate as a working-reference electrode circuit (e.g., to measure a potential, resistance, etc. across the electrode circuit). In a case where a varied or high current is passed through the composite electrode, heat can be generated at the composite electrode. The heat may be used as described elsewhere herein (e.g., to heat a polymerization reaction adjacent to the electrode). The heat may be used to aid in rinsing analytes and/or other biomolecules from a surface of the composite electrode. For example, the electrode can be heated to disassociate biomolecules from an absorptive element of the electrode.

FIG. 49 is an example of a self-contained flow array 159. The flow array may be a lateral flow array (e.g., configured for flow in a lateral direction). The flow array may be self-contained (e.g., comprise all components for function upon application of a sample). The flow array may be configured to detect one or more analytes as described elsewhere herein. The flow array may be configured to detect analytes indicative of a disease or condition. The flow array may comprise a polymer housing (e.g., a plastic housing), a metal housing, a fiber-based housing (e.g., a paper housing), or the like, or any combination thereof. The flow array housing may be transparent or translucent. For example, a subject may be able to view the contents of the flow array. The flow array may be configured to interface with one or more devices as described elsewhere herein. For example, the flow array may be configured to attach to chamber 125 to accept a sample from the chamber.

The flow array 159 may comprise tube 162. Tube 162 may be a capillary tube. Tube 162 may have a diameter of at least about 1, 5, 10, 25, 50, 75, 100, 250, 500, 750, 1,000, 2,500, 5,000, 7,500, 10,000 or more micrometers. The tube may have a diameter of at most about 10,000, 7,500, 5,000, 2,500, 1,000, 750, 500, 250, 100, 75, 50, 25, 10, 5, 1, or less micrometers. The tube may be configured to accept a sample. For example, the tube may be dipped into a sample such that the sample can flow through the tube into the flow array. In another example, a subject may flow sample directly from the subject into the tube (e.g., spit into the tube). The tube may be configured to join with a device as described elsewhere herein such as, for example tests 300 or 600. The tube may comprise stopper 163. The stopper may be plastic, metal, fiber-based, or the like, or any combination thereof. The stopper may be configured to prevent outside interaction of the contents of the flow array prior to use of the flow array. For example, the stopper can prevent reagents within the flow array from drying out. The stopper may be removed to permit a flow of fluid into the flow array. The flow array may comprise substrate 164. The substrate may be a chromatography substrate (e.g., chromatography paper, silica gel). The substrate may be configured to pull a liquid from tube 162 across the other elements of the flow array.

The flow array may comprise reagents 160 and 161. The reagents may be liquid reagents (e.g., liquid reagents soaked in a fibrous substrate, liquid reagents within a dissolvable capsule, etc.), solid reagents (e.g., powders), or the like, or any combination thereof. The reagents may be reagents configured to perform an analyte detection reaction. For example, the reagents may be a horseradish peroxidase or alkaline phosphatase and a 3,3′,5,5′-tetramethylbenzidine (TMB) substrate. The reagents may be reagents as described elsewhere herein. The flow array may comprise dye 166. The dye may be a molecular staining dye (e.g., acridine orange, a tetrazlium salt, etc.). The molecular staining dye may be configured to stain a product of a reaction comprising reagents 161 and 160. The flow array may comprise probes 167. The probes may be capture probes. The capture probes may be molecular dye capture probes. For example, a nucleic acid sample stained with a molecular dye can be bound upon flow past the capture probes. The capture probes may comprise nucleotides, oligonucleotides, proteins, antigens, antibodies, chelating agents, or the like, or any combination thereof.

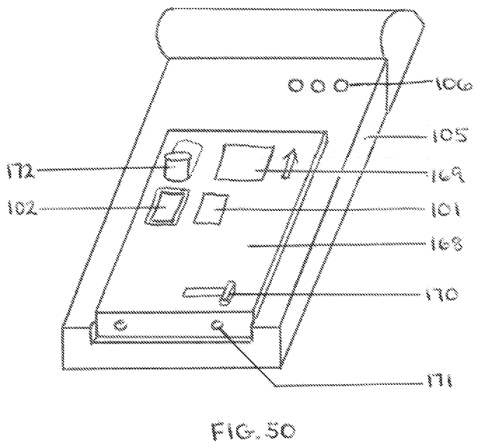

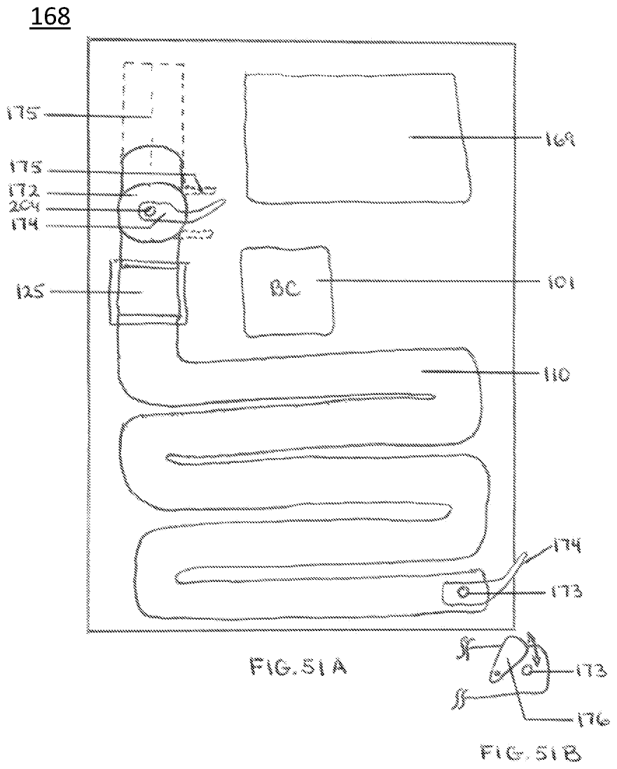

FIG. 50 shows an example of a test cartridge 168 coupled to a stand 105. The test cartridge 168 may comprise a barcode 101 as described elsewhere herein. The test cartridge may comprise a transparent window 102 as described elsewhere herein (e.g., adjacent to calibration strip 103). The test cartridge 168 may comprise information 169. The information may comprise operating instructions, lot number, expiration date, efficacy information, or the like, or any combination thereof. The instructions may comprise a code (e.g., a quick response (QR) code, a uniform resource locator (URL)) configured to direct a subject using the cartridge to an internet resource comprising information on how to use the cartridge. The information may comprise one or more pictograms, one or more written instructions, or the like, or any combination thereof. The cartridge 168 may comprise slider 170. Slider 170 may be configured to regulate at least a part of a fluid flow within the cartridge. The cartridge 168 may comprise placement indicators 171. The placement indicators may be configured to show that the cartridge is correctly inserted into the stand. The placement indicators may comprise raised elements (e.g., bumps), text elements (e.g., text indicating the direction of placement), or the like, or any combination thereof. The cartridge 168 may comprise bottle 172. The bottle may be a sample bottle as described elsewhere herein. The bottle may be a reagent bottle as described elsewhere herein. The bottle may be a bottle as described in, for example, FIGS. 56-62 and 77-79. The bottle may be configured to screw into a port of the cartridge. The port may be configured to accept multiple bottle types (e.g., sample bottles, reagent bottles). The stand 105 may be a stand as described elsewhere herein (e.g., comprising status indicators 106). The bottle may have a volume of at least about 0.01, 0.05, 0.1, 0.2, 0.3, 0.4, 0.5, 0.6, 0.7, 0.8, 0.9, 1.0, 1.1, 1.2, 1.3, 1.4, 1.5, 2.0, 2.5, 5.0, 7.5, 10.0, or more milliliters. The bottle may have a volume of at most about 10.0, 7.5, 5.0, 2.5, 2.0, 1.5, 1.4, 1.3, 1.2, 1.1, 1.0, 0.9, 0.8, 0.7, 0.6, 0.5, 0.4, 0.3, 0.2, 0.1, 0.05, 0.01, or less milliliters. The sample may be at a pH of at least about 0.5, 1, 1.5, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, or more. The sample may have a pH of at most about 13.5, 13, 12.5, 12, 11.5, 11, 10.5, 10, 9.5, 9, 8.5, 8, 7.5, 7, 6.5, 6, 5.5, 5, 4.5, 4, 3.5, 3, 2.5, 2, 1.5, 1, or less. The sample may have a pH as defined by any two of the proceeding values. For example, the sample may have a pH from about 4.0 to about 6.0.

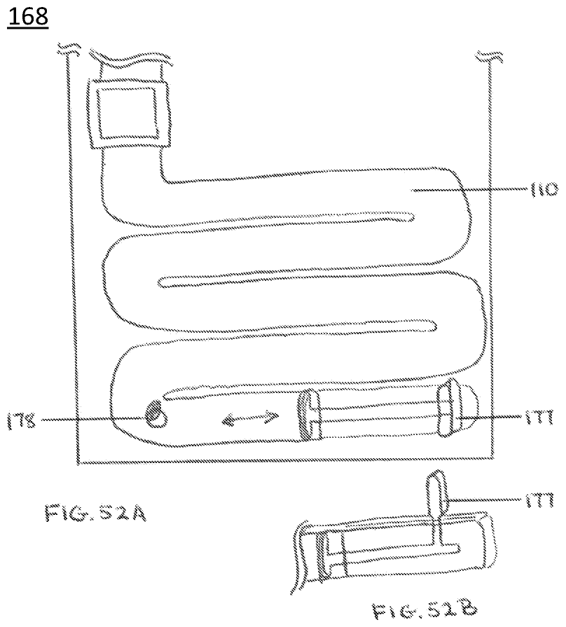

FIGS. 51A-51B are a top down view of the internal fluid paths and components of test cartridge 168, including bottle 172. The test cartridge 168 may comprise a reaction and/or detection chamber 125 as described elsewhere herein. The reaction and/or detection chamber may be optically addressable from outside of the test cartridge (e.g., a transparent window is positioned above the chamber). The test cartridge 168 may be configured to flow liquid from the bottle 172 to chamber 125. The liquid may then flow from chamber 125 to fluidic channel 110. The liquid in fluidic channel 110 may be waste liquid (e.g., liquid already used in chamber 125).

Bottle 172 may be guided by guide tracks 175. A plurality of bottles can be placed in the guide tracks (e.g., sample bottle, reagent bottles, wash bottles, etc.). For example, the guide tracks can hold a plurality of bottles, and a subject can slide the bottle down the guide tracks, insert the bottle into the port, empty the bottle, and remove the bottle from the opening in the tracks. The bottle may comprise hole 204. The hole may be a vent hole (e.g., a hole configured to prevent a vacuum from forming within the bottle), a fill hole (e.g., a hold configured to accept a liquid or gas into the bottle), or the like, or any combination thereof. The hole may be covered by seal 174. The seal may be an adhesive seal (e.g., a tape) a mechanical seal (e.g., a plug, a movable cover), or the like, or any combination thereof. The seal may be disposable (e.g., configured to be removed and disposed of) or reusable (e.g., configured to be removed and replaced on the hole). The seal may be a sliding seal such as, for example, sliding seal 176 of FIG. 51B. Hole 173 of FIG. 51B may be configured as a pressure release hole. For example, the hole can be uncovered to permit a flow of fluid through channel 110.

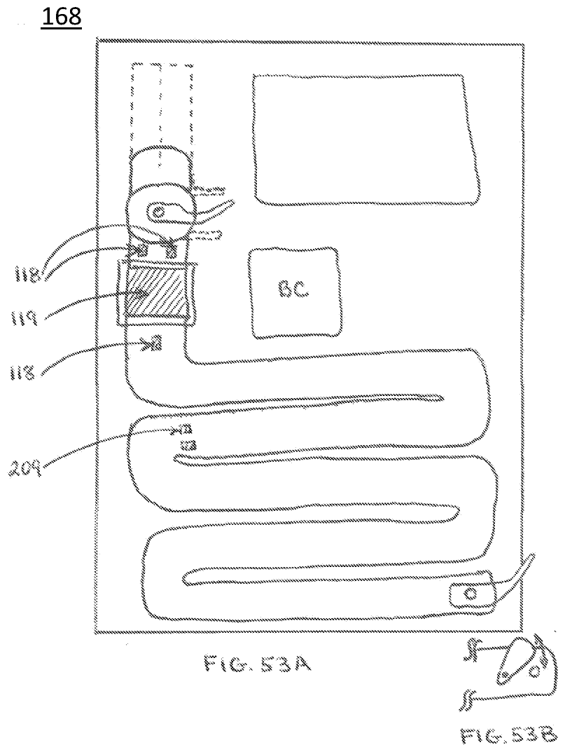

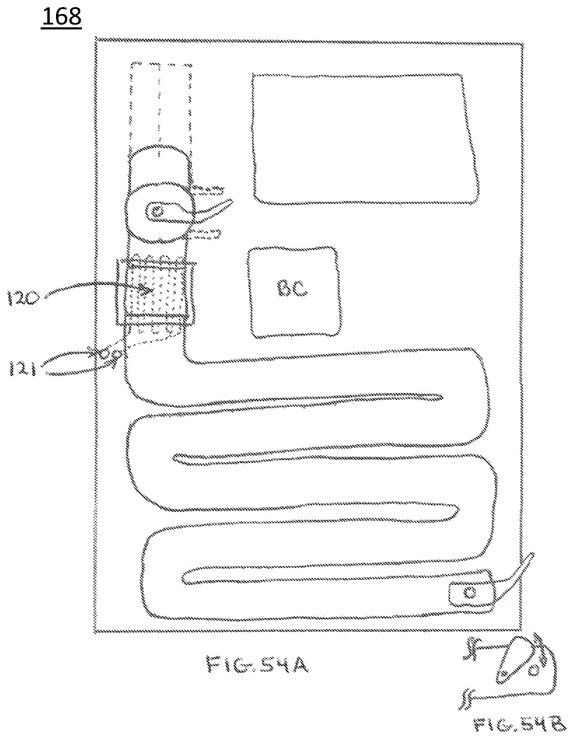

FIGS. 52A-52B are an example of an optional slider 177. The optional slider may be attached to a plunger as shown in FIG. 52. The plunger may be configured to be operated by a subject. For example, a subject can slide the slider along its channel to move the connected plunger. The slider may be configured to assist in liquid movement within the channel 110 and/or the chamber 125. For example, a subject can push the slider out to pull liquid down into the channel. The slider may be configured to exert sufficient force to provide liquid movement even in the presence of bubbles within the test cartridge. For example, a bubble can be blocking fluid flow in the channel, and the slider can provide sufficient force to move the bubble and reestablish fluid flow in the channel. The test cartridge may comprise one or more valves 178. The one or more valves may be one or more one-way valves (e.g., permitting flow into the channel or out of the channel, but not both). The one or more valves may be configured to correct a pressure imbalance within the channel. For example, as fluid flows into the channel, the valve can release gas from further on in the channel to permit the continued flow of fluid. In another example, the valve can release gas pressurized by a movement of the slider. FIGS. 53A-53B are an example test cartridge 168 comprising electrodes 118. The electrodes may be electrodes as described elsewhere herein (e.g., configured as lysis electrodes, fluidic movement detection electrodes, fluid handing electrodes, or the like, or any combination thereof). FIGS. 54A-54B are an example test cartridge 168 comprising a heating element 120. The heating element may be a heating element as described elsewhere herein such as, for example, in FIGS. 15-16.

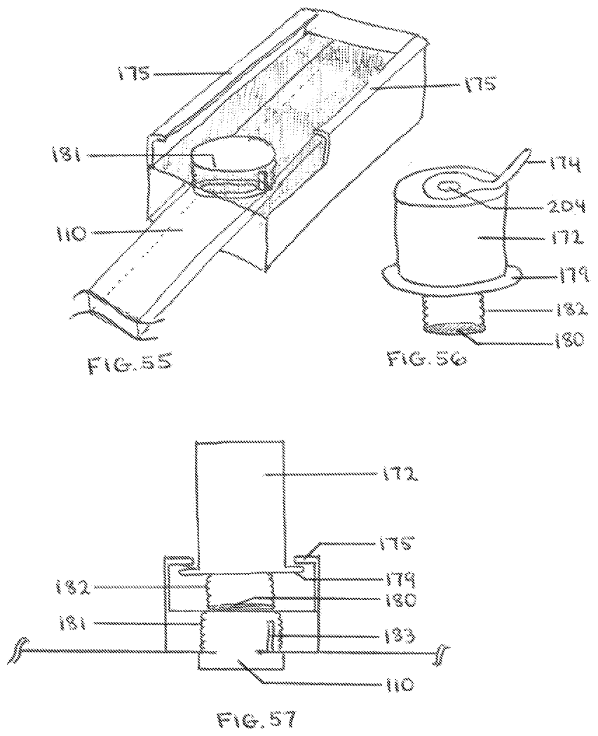

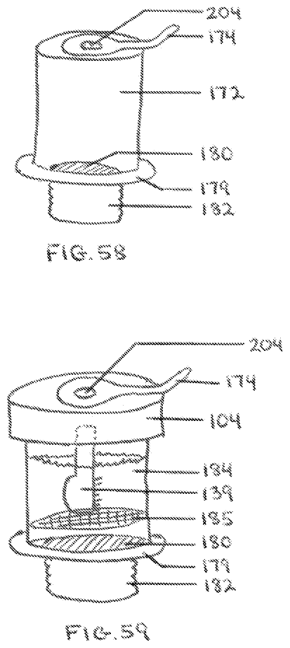

FIGS. 55 and 57 are an example of an access port 181 adjacent to guide tracks 175 of test cartridge 168. The guide track may be optional. For example, the test cartridge 168 may not comprise the guide track. The guide tracks may be configured to store one or more bottles. The guide tracks may be configured to have the bottles be removed from the guide tracks after the bottles have been used. For example, a bottle can be removed from the cutout in the guide tracks shown in FIG. 55. The guide track may be configured to accept bottles into the guide track. For example, a subject can slide a bottle into the guide track, place it into the access port, drain a liquid from the bottle, and slide it into the guide track for storage before disposal. The guide track may reduce the number of individual items disposed of after test use by retaining the used bottles.

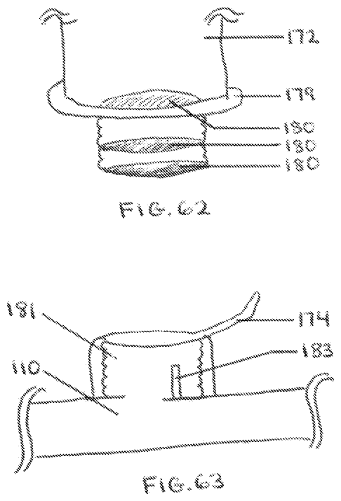

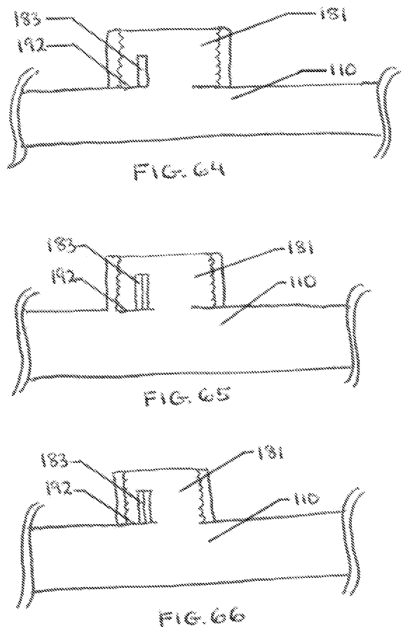

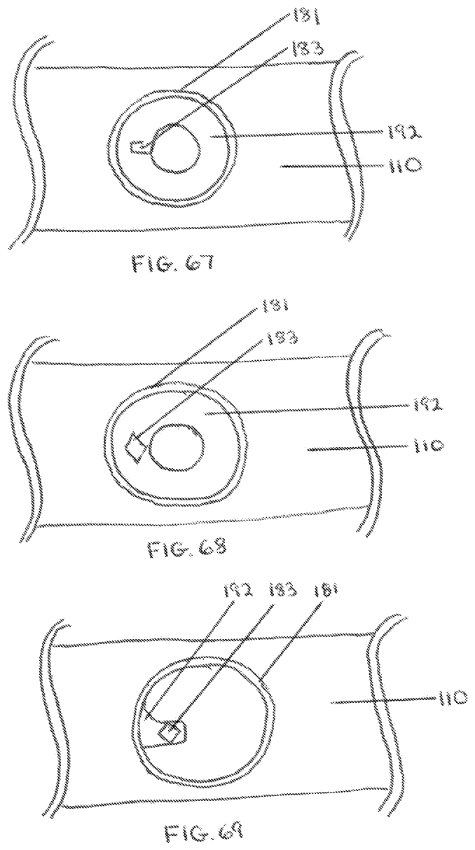

The access port may be an access port as described elsewhere herein such as, for example, FIGS. 63-69 and 80. The access port may be threaded, smooth, keyed (e.g., configured to only accept bottles with the corresponding keying), or the like, or any combination thereof. For example, the access port can be smooth and keyed. The access port may be configured to permit flow of fluids into the fluidic channel 110. For example, reagents can flow from a reagent bottle through the access port into the channel. The access port may comprise burst element 183. The burst element may be configured to puncture a seal of a bottle. The burst element may be a burst element as described elsewhere herein such as, for example, in FIGS. 63-76.

FIG. 56 is an example of a bottle 172. The bottle may be a bottle as described elsewhere herein such as, for example FIGS. 58-62 and 77-79. The bottle may comprise rigid sides or deformable sides. For example, the bottle may be squeezable by a subject to direct the contents of the bottle into the access port. The bottle may comprise plastic, a metal, or the like, or any combination thereof. For example, the bottle can be a polyethylene bottle. The bottle may comprise hole 204 as described elsewhere herein. The hole may be configured as a vent hole. The hole may be configured to accept reagents and/or samples. The addition of reagents through the hole may enable the use of custom assays on the test cartridge. The hole may be sealed with 174 as described elsewhere herein. The bottle may comprise lip 179. The lip may be configured to secure the bottle within the guide tracks 175 as shown in FIG. 57. The lip may comprise the same material as the bottle. The lip may comprise a different material from the bottle. The bottle may be configured to interface with the access port. For example, a bottle configured to interface with a threaded access port can comprise threads 182. In another example, a bottle configured to interface with a keyed access port can comprise a key pattern complimentary to the pattern of the access port. The bottle may comprise a seal 180. The seal may be a seal as described elsewhere herein. The seal may be configured to keep the contents of the bottle within the bottle until the seal is broken by insertion of the bottle into the access port. For example, a seal can keep the contents of a reagent bottle within the bottle until the seal is punctured by a burst element. The seal may be placed throughout the bottle. The seal may be placed at the bottom of the bottle (e.g., FIG. 56), higher in the bottle (e.g., FIG. 58), or the like. A plurality of seals may be placed within the bottle (e.g., FIG. 62). At least about 1, 2, 3, 4, 5, 6, 7, 8, 9, 10, or more seals may be placed within the bottle. At most about 10, 9, 8, 7, 6, 5, 4, 3, 2, or less seals may be placed within the bottle.

FIG. 59 is an example of a sample bottle. The sample bottle may be configured to accept a sample from a user. For example, a user can place saliva through hole 204 directly into the sample bottle. In another example, the user can use a swab (such as the swab of FIG. 18) to collect the sample. In this example, the user can break head 139 off of the swab and place it into the sample bottle. The sample bottle may comprise a sample cap 104. The sample cap may be removable. The sample bottle may comprise sample solution 184. The sample solution may comprise one or more buffers (e.g., cell lysis buffers, preservation buffers, etc.), one or more solvents (e.g., water, alcohols), one or more surfactants, one or more ionic species (e.g., salts), or the like, or any combination thereof. The sample solution may be configured to process the sample in preparation for a later reaction. For example, the sample solution can lyse cells of the sample to expose nucleic acid molecules for later amplification. The sample bottle may comprise mesh 185. The mesh may be configured to permit a fluid and/or gas flow through the sample bottle but restrict solid flow. For example, the mesh can prevent the head from falling into the access port. The mesh may be a plastic mesh, a metal mesh, a fibrous sheet (e.g., a liquid permeable paper), or the like, or any combination thereof. For example, the mesh can be a plastic mesh with paper components filling the holes of the mesh. The sample bottle may comprise one or more seals 180, as described elsewhere herein.

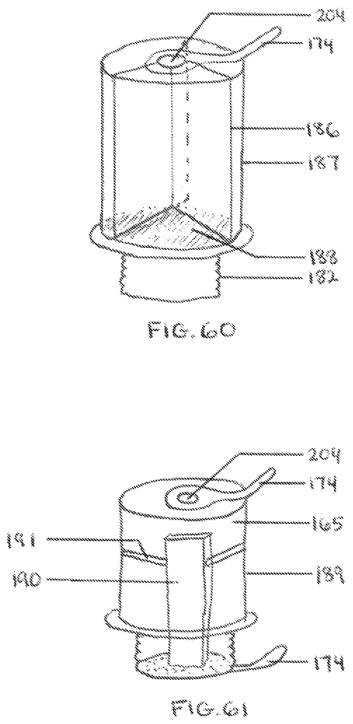

FIG. 60 is an example of a bottle 187 comprising a plurality of chambers. The bottle may be a bottle as described elsewhere herein with an addition of one or more panels 186. Panels 186 may separate the interior of the bottle 187 into a plurality of chambers. The bottle may have at least about 1, 2, 3, 4, 5, 6, 7, 8, 9, 10, or more chambers. The bottle may have at most about 10, 9, 8, 7, 6, 5, 4, 3, 2, or less chambers. Each chamber of the plurality of chambers may contain a different material. For example, each chamber can have different reagents. In another example, one chamber can hold the sample while other chambers hold reagents. Each chamber the plurality of chambers may comprise a different seal 188. Each chamber comprising a different seal may enable the chambers to be opened and the contents flowed through the access port at different times. For example, the seal of a first chamber comprising a washing reagent can be broken to flow the washing reagent out of the bottle, and subsequently the seal of a second chamber comprising an analysis reagent can be broken and the analysis reagent can be flowed out of the bottle.

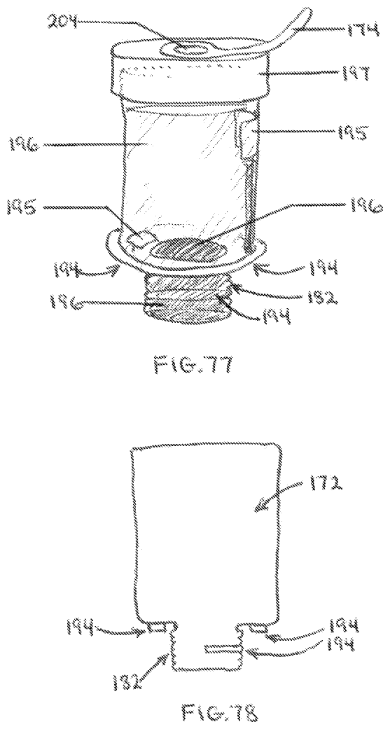

FIGS. 70-71 are example side and bottom views of a bottle 187 comprising a plurality of interior chambers. In this example, there can be three interior chambers. The seal of each interior chamber may be attached to tabs A, B, and C. Each of tabs A, B, and C may be different length tabs. The tabs may be affixed to the seal such that the seal is configured to burst before the tab is removed from the seal. For example, a tab can be part of a plastic plug seal. The tabs may be configured to impact a burst element within the access port as a user inserts the bottle 187. For example, as a user screws the bottle into the access port, the longest tab can impact the burst element when the bottle has been sufficiently screwed in. When a tab impacts the burst element, the tab may remove the associated seal, permitting fluid flow from the bottle through the access port. For example, tab A can impact the bursting element and the associated seal can be broken from the bottle. As the bottle continues to be inserted into the access port, the other, shorter tabs can impact the burst element in order of their height, similarly removing the associated seal and releasing the contents of the camber into the access port. For example, tab A can impact the burst element, then tab B, then tab C.

FIG. 61 is an example of a test bottle 189. The test bottle may comprise a gas filled (e.g., air filled) chamber 165. The test bottle may comprise a test substrate 190. The test substrate may be a porous substrate (e.g., a paper-based substrate, a porous polymer substrate), a solid substrate (e.g., silica gel on plastic), or the like, or any combination thereof. The test substrate may be configured as a chromatography substrate. For example, the substrate can be configured to separate components of a solution based off of an association strength with the substrate. For example, the test bottle can have a solution comprising a plurality of analytes placed in contact with the base of the chromatography substrate and the analytes can be separated by their affinity for the substrate as the solution wicks up the substrate. The chromatography substrate may be an affinity chromatography substrate (e.g., silica gel, a substrate infused with antibodies/antigens, a substrate comprising one or more chelating agents, etc.), a size exclusion chromatography substrate (e.g., molecular sieves), or the like, or any combination thereof. The test bottle may comprise seal 174. The seal may be removed before inserting the test bottle into the access port. The seal may be configured to be removed by a subject. The seal may be configured to be punctured, such as by a burst element.

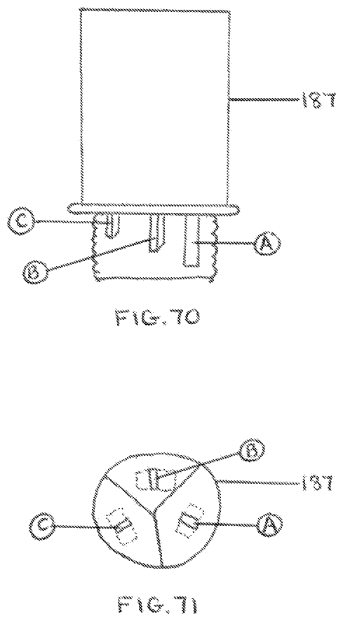

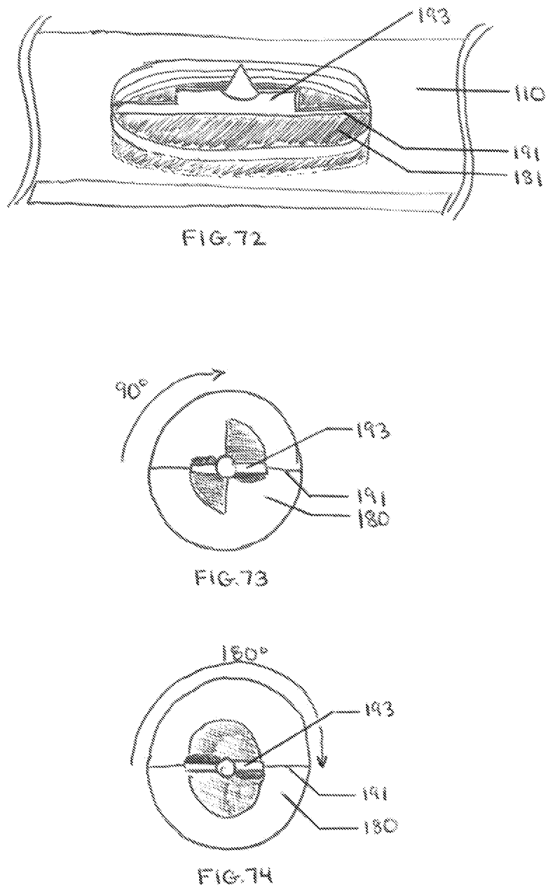

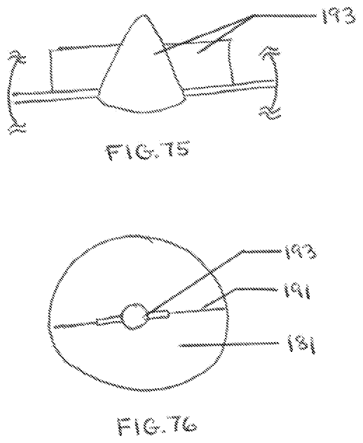

FIG. 63 is an example side view of an access port 181 comprising a burst element 183. The access port may be covered by seal 174 prior to operation. A user may remove seal 174 from the access port. The seal may be configured to break upon insertion of a bottle to the access port. The seal may be a seal as described elsewhere herein. The burst element may be configured to burst a seal of a bottle after the bottle is securely inserted into the access port. For example, the burst element may be short enough that it does not burst the seal until the bottle has a liquid tight seal with the access port (e.g., is screwed into the access port). After the seal of the bottle is ruptures, the fluid within the bottle may flow into channel 110. The burst element may be a burst element as described in FIGS. 64-66. FIGS. 64-66 are example side views of some embodiments of access ports 181 comprising burst elements 183. FIGS. 67-69 are example top-down views of some embodiments of access ports 181 comprising burst elements 183. The burst element may be attached to the access port by lip 192. In some embodiments, the opening from the access port to the channel 110 may be symmetrical (e.g., FIGS. 64-65) or asymmetrical (e.g., FIG. 66). The burst elements may be of any shape. The burst elements may be rectangular (e.g., FIG. 67), diamonds (e.g., FIG. 68), any other geometric shape (e.g., square, FIG. 67), spiral, blade shaped, curved, cones, or the like, or any combination thereof.

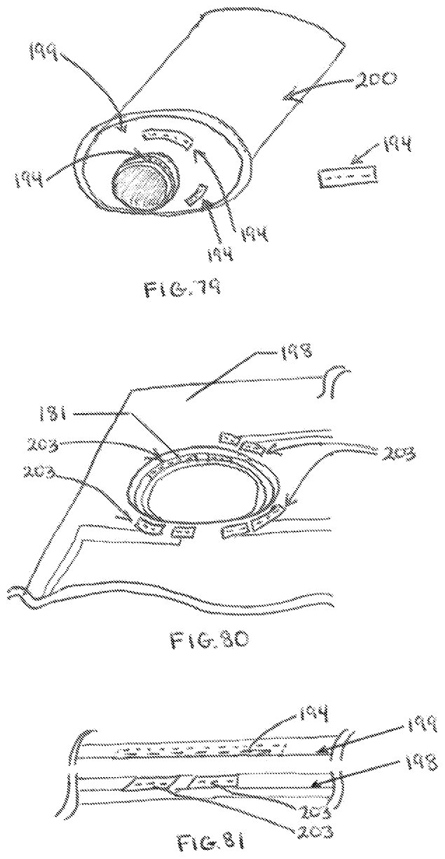

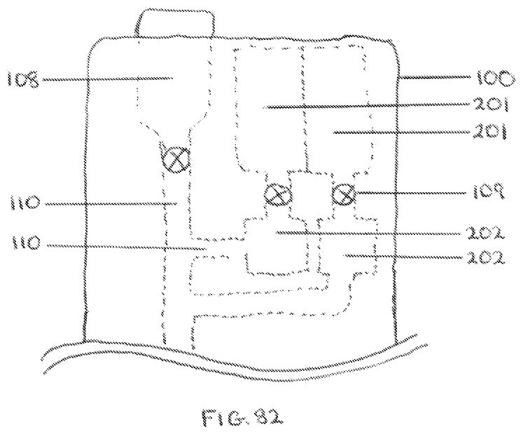

FIG. 72 is an example of an access port 181 comprising a burst blade 193. The burst blade 193 may be configured to burst a seal of a bottle. The blade may be a plastic blade, a metal blade, a wooden blade, or the like, or any combination thereof. The blade may be suspended in the center of the access port by supports 191. The supports may be of a same material as the blade. The supports may be of a different material from the blades. The blade may be configured to puncture the seal of a bottle (e.g., have a sharp component such as the cone portion of FIG. 75-76). The blade may be configured to tear the seal of a bottle (e.g., comprise an element such as the rectangular portion of FIG. 75-76). FIGS. 73-74 are examples of a bottom-up view of a seal 180 being burst by burst blade 193. In FIG. 73, the bottle may have been rotated 90 degrees from the point of initial penetration of the seal (e.g., rotated while the bottle was being screwed into the access port), and the burst blade may have generated hole in the seal represented by the shaded regions of the seal. In FIG. 74, the bottle may have been rotated 180 degrees from the point of initial penetration of the seal to generate the full hole in the seal represented by the shaded region. FIGS. 75-76 are examples of different views of burst blades 193.

FIG. 77 is an example of an electrophoresis bottle. The electrophoresis bottle may comprise electrodes 195. The electrodes may be electrodes as described elsewhere herein. The electrodes may be configured to apply a bias across medium 196. Medium 196 may be a gel (e.g., an electrophoresis gel), sieves (e.g., molecular sieves), or the like, or any combination thereof. The electrodes may apply a bias across the medium to perform an electrophoretic separation. For example, sample comprising analytes can flow from the bottom of the bottle into contact with the medium, and the electrodes can perform a gel electrophoresis on the analytes to separate the analytes. The electrophoresis bottle may be transparent for visualization of the electrophoretic separation performed within the bottle. The electrophoresis bottle may comprise electrical elements 194. The electrical elements may be electrical elements as described elsewhere herein. The electrical elements may connect the electrodes to power and/or control circuitry contained within the stand. For example, the electrical elements can electrically interface with pads on a test cartridge to enable biasing of the electrodes. A sample may be applied to the top of the medium through hole 204. The sample may then be separated within the medium with aid of the electrodes before being flowed into the channel.

FIG. 78 is an example bottle 172 comprising electrical elements 194. The bottle may be a bottle as described elsewhere herein. The electrical elements may be electrical elements as described elsewhere herein. The electrical elements may be configured to permit detection of an insertion of the bottle (e.g., the elements complete a circuit with pads adjacent to the access port of a test cartridge), of a depth of insertion of the bottle (e.g., the elements complete a circuit with pads within the access port to detect how deep the bottle has been inserted), or the like, or any combination thereof. The electrical elements may permit an application of power to the bottle (e.g., the elements are connected to electrodes internal to the bottle and serve as a bridge from the test cartridge to the bottle), a read of information from the bottle (e.g., a measurement of internal electrodes to determine how full a bottle is), or the like, or any combination thereof.

FIG. 79 is an example bottom view of a bottle 200 comprising electrical elements 194. The bottle may be a bottle as described elsewhere herein such as, for example, bottle 172. The bottle 200 may comprise a bottom 199. The bottom may be decorated with one or more electrical elements 194. The electrical elements may be applied to an insertion portion (e.g., a threaded portion) of the bottle. Electrical elements 194 may comprise conductive elements, resistive elements, capacitive elements, semiconductor elements, or the like, or any combination thereof. The conductive elements may comprise metals (e.g., copper, silver, gold, iron), alloys (e.g., brass, steel), organic conductors (e.g., graphite impregnated paper), or the like, or any combination thereof. The resistive elements may be ceramics, organic resistors, or the like, or any combination thereof. The capacitive elements may be ceramics, electrolytic capacitors, film capacitors, or the like, or any combination thereof. The electrical elements comprise an adhesive. For example, the electrical element can comprise an adhesive to hold the electrical element to the bottle. Values of properties of the electrical element (e.g., resistivities, capacitances) can be related to properties of the bottle 200. For example, each bottle can comprise electrical elements with different values of resistance, such that a measurement of a particular resistance can indicate which bottle is being applied to the test cartridge. Information such as bottle type, position, lot number, expiration date, or the like, or any combination thereof, can be related to the value of the property of the electrical element.

FIG. 80 is an example of an access port 181 on a surface 198 of a test cartridge with adjacent electrical element detectors 203. Each electrical element detector may comprise a pair of electrodes. The pair of electrodes may be in electronic communication with a processor (e.g., a processor of the stand). The pair of electrodes may be electrodes as described elsewhere herein (e.g., metal electrodes, graphitized paper electrodes, etc.). The electrical element detectors may be of the surface 198 of the test cartridge, within the access port 181, or the like, or any combination thereof. The test cartridge may comprise at least about 1, 2, 3, 4, 5, 6, 7, 8, 9, 10, or more electrical element detectors. The test cartridge may comprise at most about 10, 9, 8, 7, 6, 5, 4, 3, 2, or fewer electrical element detectors. The electrical element detectors may be configured to be bridged by an electrical element 194 attached to a bottle 200. For example, electrical elements on the surface of the cartridge can be bridged by a bottle inserted into the access port sufficiently far to contact the electrical elements on the bottom of the bottle with the electrical element detectors. In another example, the electrical elements on the inserted portion of the bottle can be detected by electrical element detectors in the access port. The electrical element detectors may be configured to permit measurement of the properties of the electrical element. For example, for an electrical element comprising a resistive element, the electrical element detectors can enable measurement of the resistance of the electrical element. In this example, the value of the resistance can be correlated with the position of the electrical element, providing information about the exact orientation of the bottle, the contents of the bottle, or the like. The electrical element detectors may be configured to provide power and/or measure one or more electrical signals through the electrical element. For example, conductive electrical elements connected to electrodes within an electrophoresis bottle can contact electrical element detectors and power for the electrodes can be conducted from the chip to the electrodes. FIG. 81 is an example of an electrical element 194 approaching but not yet bridging electrical element detectors 203. In this example, a signal is not recorded from the electrical element detectors because the electrical element has not yet bridged the electrical element detectors.

### Computer Systems

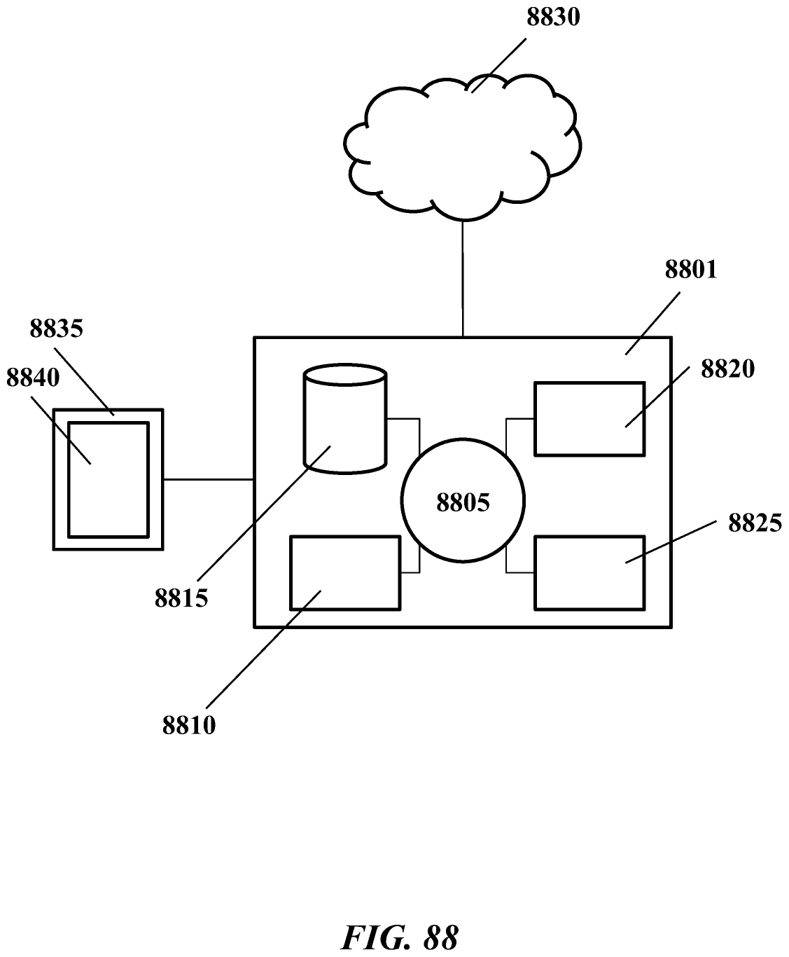

The present disclosure provides computer systems that are programmed to implement methods of the disclosure. FIG. 88 shows a computer system 8801 that is programmed or otherwise configured to control elements of the devices and methods described herein. The computer system 8801 can regulate various aspects of the present disclosure, such as, for example, applied temperature, applied bias voltages and patters, reaction times, etc. The computer system 8801 can be an electronic device of a user or a computer system that is remotely located with respect to the electronic device. The electronic device can be a mobile electronic device. The computer system may be a microprocessor

The computer system 8801 includes a central processing unit (CPU, also “processor” and “computer processor” herein) 8805, which can be a single core or multi core processor, or a plurality of processors for parallel processing. The computer system 8801 also includes memory or memory location 8810 (e.g., random-access memory, read-only memory, flash memory), electronic storage unit 8815 (e.g., hard disk), communication interface 8820 (e.g., network adapter) for communicating with one or more other systems, and peripheral devices 8825, such as cache, other memory, data storage and/or electronic display adapters. The memory 8810, storage unit 8815, interface 8820 and peripheral devices 8825 are in communication with the CPU 8805 through a communication bus (solid lines), such as a motherboard. The storage unit 8815 can be a data storage unit (or data repository) for storing data. The computer system 8801 can be operatively coupled to a computer network (“network”) 8830 with the aid of the communication interface 8820. The network 8830 can be the Internet, an internet and/or extranet, or an intranet and/or extranet that is in communication with the Internet. The network 8830 in some cases is a telecommunication and/or data network. The network 8830 can include one or more computer servers, which can enable distributed computing, such as cloud computing. The network 8830, in some cases with the aid of the computer system 8801, can implement a peer-to-peer network, which may enable devices coupled to the computer system 8801 to behave as a client or a server.

The CPU 8805 can execute a sequence of machine-readable instructions, which can be embodied in a program or software. The instructions may be stored in a memory location, such as the memory 8810. The instructions can be directed to the CPU 8805, which can subsequently program or otherwise configure the CPU 8805 to implement methods of the present disclosure. Examples of operations performed by the CPU 8805 can include fetch, decode, execute, and writeback.

The CPU 8805 can be part of a circuit, such as an integrated circuit. One or more other components of the system 8801 can be included in the circuit. In some cases, the circuit is an application specific integrated circuit (ASIC).

The storage unit 8815 can store files, such as drivers, libraries, and saved programs. The storage unit 8815 can store user data, e.g., user preferences and user programs. The computer system 8801 in some cases can include one or more additional data storage units that are external to the computer system 8801, such as located on a remote server that is in communication with the computer system 8801 through an intranet or the Internet.

The computer system 8801 can communicate with one or more remote computer systems through the network 8830. For instance, the computer system 8801 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 8801 via the network 8830.

Methods as described herein can be implemented by way of machine (e.g., computer processor) executable code stored on an electronic storage location of the computer system 8801, such as, for example, on the memory 8810 or electronic storage unit 8815. The machine executable or machine-readable code can be provided in the form of software. During use, the code can be executed by the processor 8805. In some cases, the code can be retrieved from the storage unit 8815 and stored on the memory 8810 for ready access by the processor 8805. In some situations, the electronic storage unit 8815 can be precluded, and machine-executable instructions are stored on memory 8810.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 8801, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 8801 can include or be in communication with an electronic display 8835 that comprises a user interface (UI) 8840 for providing, for example, an indication of a presence of an analyte on a screen of a smartphone. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 8805. The algorithm can, for example, control the temperature and time of an amplification reaction, control a detection electrode, etc.

### EXAMPLES

The following examples are illustrative of certain systems and methods described herein and are not intended to be limiting.

### Example 1—Example Assay

An example assay of a sample implemented on a test cartridge (e.g., cartridge 300, 600, 168, etc.) can be configured with pre-spotted graphite impregnated paper electrodes. The pre-spotting can comprise target DNA forward primer for four genomic regions of interest, 2 genomic locations of COVID 19, a positive control spiked into the sample, and a natural mitochondrial DNA control. The graphite impregnated paper electrode can be configured to geographically isolate nucleic acid probes that may be applied to and attached to the surface of the graphite impregnated paper electrode as well as contain any assay products produced on the attached probes. The probes may be pre-deposited on the graphite impregnated paper electrode. The graphite impregnated paper electrode may be patterned (e.g., with valleys) in order to isolate the individual probe positions from one another.

A saliva and oral sample can be taken by a user and caped in the sample reservoir of the test cartridge. The sample reservoir can contain a high salt and low surfactant concentration lysis buffer and protease K to destroy proteins and cellular components.

The test cartridge can be inserted into a control module or stand and powered on. The control module or stand can then energize lysis electrodes at 2V with 500 millisecond switching to lyse the cells of the sample. The control module or stand can also energize the internal heater wire to 1.2V and the graphite impregnated paper electrode to 1.2V. Subsequently to the energizations, a timer within the control module or stand is activated. The bias of the graphite impregnated paper electrode can improve the adhesion of nucleic acids to the paper at both high and low salt concentrations.

The user can hand mix the lysis buffer and swab in the sample reservoir for 30 seconds to aid in the lysis of the sample cells and prepare the sample for testing.

The safety valve of the test cartridge can be opened and the user can squeeze the sample down into the reaction and/or detection chamber of the cartridge. The DNA/RNA analytes can be collected at the graphite impregnated paper electrode. The methods and devices herein can permit use of a harsher lysis buffer because of the extensive rinsing into waste that is possible. For example, the sample can be rinsed while the lysis electrodes, graphite impregnated paper electrode, and heater are all activated.

The user can open the rinse reservoir and squeeze a low salt rinse solution through the reaction and/or detection chamber (e.g., a 150 mM NaCl, minimally buffered, pH 7 solution). At this operation, the lysis electrode can be disabled while the graphite impregnated paper electrode is enabled to retain the charged sample within the chamber as it is heated by the heater. A waste sensing electrode at the end of the chamber can sense the waste flowing through the chamber

The user can open the amplification and labeling reservoir and squeeze the contents down and into the reaction and/or detection chamber. The amplification reservoir can contain combined 37° C. isothermal reverse transcriptase, Avian Myeloblastosis Virus Reverse Transcriptase (AMV RT), 5′ biotinylated target, spike, and mitochondrial reverse primers and standard forward primers, spike DNA, and pH sensitive colorimetric reporting dye. The contents can be configured to stoichiometrically favor reverse primers. The pH sensitive colorimetric reporting dye can turn pink to yellow to indicate enzymatic activity resulting from template extension. During this operation the lysis electrode can be off, the graphite impregnated paper electrode can be slowly switched between positive and negative voltages (e.g., from −500 mV to 500 mV), and the heater can be on per a timer. The switching can attract and repel DNA and charged proteins, thus speeding up a reverse transcription reaction and the subsequent DNA amplification and labeling reaction.

Alternately, the previous operation may be split into two steps—the sequential application of RT enzyme and then application of the RPA enzyme system.

The user can wait 20 minutes. Subsequently to waiting, the user can view the detection window to view any color changes (e.g., pink to yellow) at pre-deposited points on the graphite impregnated paper electrode. A single smartphone photo can be taken with app software of the detection window/graphite impregnated paper electrode, color calibration strip, and the barcode. The synthesis of DNA can generate a sufficient number of H+ ions to impart a pH change in a region of DNA amplification, which can be detected by a pH indicator. Applying a voltage to the graphite impregnated paper electrode can reduce the time to see a colorimetric pH reporting agent by increasing the concentration of the H+ ions and/or the reporting agent.

The user can open a rinse reservoir and squeezes it past the detector window with the lysis electrodes off, the graphite impregnated paper electrode set to 1.2V, and the heater on. The graphite impregnated paper electrode can be energized to keep the sample and probe DNA on the paper surface of the graphite impregnated paper electrode during the rinse.

The user can open a horseradish peroxidase (HRP) and 3,3′,5,5′-tetramethylbenzidine (TMB) dual reservoir and squeezes it to the detection window with the lysis electrode off, the graphite impregnated paper electrode on, and the heater on per a timer. The detection assay can take place with a low applied voltage on the graphite impregnated paper electrode to keep the extension products above stationary recognition probes. The streptavidin conjugated HRP enzyme can then bind to the biotin and enzymatically oxidize the TMB to deposit a green color onto the substrate. For example, the electrodes can be maintained at a voltage (e.g., less than 100 mV) to prevent oxidation of the clear TMB substrate in the presence of HRP. In this example, the positive voltage can assist in holding negatively charged analytes and probes in place adjacent to the membrane.

The user can wait 10 minutes and views the detection window to view color changes at pre-deposited points on the graphite impregnated paper electrode, where the lysis electrode, graphite impregnated paper electrode, and heater are off. A single smartphone photo is taken of the detection window/graphite impregnated paper electrode, color calibration strip, and barcode. Cell phone application software creates reports that are automatically reported to a programmed list of recipients (e.g., the user, the user's employer, the user's doctor). If negative, the cell phone screen is turned bright green with the date and time stamped in large lettering. If the test is positive, the cell phone screen is turned red with large Lettering with date and time, the recipients are notified, and the user is encouraged to isolate themselves and get tested at a laboratory. The total time of this process can be 35 minutes with a total hands on time for the user of less than 5 minutes.

### Example 2—Example Membrane Functionalization with Oxides

A cellulose membrane can comprise hydroxyl groups on the surface of the membrane. The hydroxyl groups can, via a salt bridge (e.g., a magnesium ion), bind the negatively charged backbone of a nucleic acid analyte to the cellulose. An improvement to the binding capability of the cellulose membrane can be to functionalize the cellulose with silica (e.g., silicon dioxide). The silica may comprise hydroxyl groups which can be linked to the cellulose via a dehydration reaction, thereby binding the silica to the surface of the membrane. Similar chemistries can be used to link silica to a nylon membrane. The dehydration reaction can be initiated by chemical reagents (e.g., acids), light, or a combination thereof.

The silica can be in the form of silica nanoparticles, which can present hydroxyl groups for binding of nucleic acid analytes to the membrane. Additionally, the use of a silica surface can provide a well understood binding surface, which in turn can use high performance binding, washing, and de-binding chemistries. For example, nucleic acid quantification techniques can be used as shown in “Low concentration DNA extraction and recovery using a silica solid phase” by Katevatis et al., DOI number 10.1371/journal.pone.0176848 or “DNA Adsorption to and Elution from Silica Surfaces: Influence of Amino Acid Buffers” by Vandeventer et al., DOI number 10.1021/jp405753m, the disclosures of each of which are incorporated herein by reference.

A membrane may be prepared via a sol-gel process. For example, tetraethyl orthosilicate (TEOS) can be hydrolyzed in a mixture of ethanol (e.g., common solvent), water, ammonia (e.g., catalyst), and a surfactant. In this example, 10 mL of ethanol, 3 mL of deionized water, 1 mL TEOS, and 2.5 g non-ionic surfactant can be mixed and stirred for 30 minutes. Ammonium hydroxide can be added dropwise to control the pH of the reaction mixture. Upon addition of the ammonium hydroxide, the solution can initially remain clear and slowly increase in turbidity during the formation of the silica. The formation of silica can take approximately 2 hours to complete. To incorporate the silica with the membrane, the membrane can be included in the sol-gel reactor to form silica on the membrane. The silica may be removed from the reactor, processed (e.g., by removal of the solvents, by calcination, etc.), and applied to the membrane. The silica can then be linked to the membrane as described elsewhere herein. The surfactant used and the amount of surfactant can be changed based on a predetermined size of the silica to be produced. An example of a sol-gel method without a membrane can be found in “Influence of silica nanoparticles on the properties of cellulose composite membranes: a current review” by Dlomo et al., Cellulose Chem. Technol., 54 (7-8), 765-775(2020), the disclosure of which is incorporated by reference in its entirety.

Another example of a method for treating a membrane with a second material may be to functionalize the membrane with functional groups configured to bind to the second material. For example, a cellulose membrane can be exposed to silanes bearing functional groups configured for binding of the second material. The silanes can bind to the hydroxyl groups of the cellulose, which can in turn present the functional groups for binding the second material. For example, an amino-silane (e.g., aminopropyl-trimethoxysilane, aminopropyl-trichlorosilane, etc.) can be bound via the silane groups to a cellulose membrane, thus leaving exposed amine groups for binding nanoparticles to the membrane. In this example, the hydroxyl groups of silica nanoparticles can also be functionalized with amino-silanes, and the amine functionalized silica and cellulose can be bound together by exposure to high-energy light (e.g., from a Stratalinker® UV crosslinker). Such functionalization can be controlled to maintain un-reacted hydroxy groups on the surface of the silica to in turn bind with the analyte. Using such functionalization chemistries, silica (or other oxides) of arbitrary size or shape can be bound to the membrane.

The membrane may be functionalized such that the surface has a positive charge, a negative charge, or be charge neutral. An example of a functionalization that renders a surface of the membrane with a positive charge may be incorporation of a positive or partially positive charged functional group. For example, the chlorine of 2-chlorotriethylamine can react with a surface hydroxide of cellulose to bind the amine functionality and provide a partial positive charge on the surface of the cellulose. By including a positive or partially positive surface on the membrane, a lower salt or alcohol concentration may be used in the binding of the analyte to the membrane. This may result in more mild conditions for the processing of the analyte.

Another method of functionalizing a membrane may be to apply a suspension of a second material to the membrane. The suspension may be a slurry comprising the second material, a cream comprising the second material, or the like. The suspension may be applied by spin coating, dip coating, doctor blading, drop casting, centrifugal casting, chemical solution deposition, or the like, or any combination thereof. The suspension may then be processed to remove solvents and leave behind the second material. For example, a suspension of titanium oxide in water can be baked in a vacuum oven to remove the water. The second material may then be attached to the membrane as described elsewhere herein. For example, the second material can be exposed to light to bind it to the membrane. Such a suspension-based application of the second material may provide improvements in cost and manufacturability.

The membrane may be functionalized as to bind a plurality of analytes from a sample to the membrane. For example, the membrane can non-specifically bind all or substantially all of the nucleic acids from a sample. To determine a presence of an analyte on the membrane, a solution comprising reagents specific for the analyte can be added to the membrane, and the reagents can be used to determine the presence or absence of the analyte. For example, a solution comprising a specific nucleic acid complementary to an analyte comprising a signaling moiety can be flowed onto the membrane, and a signal can be detected from the complementary nucleic acid. A presence of a plurality of analytes can be determined by using a solution comprising a plurality of reagents configured to generate a signal indicative of a presence of an analyte of the plurality of analytes. For example, a solution configured to provide an indication of a presence of 6 different analytes can be flowed into contact with a membrane configured to bind all analytes from a solution to the membrane. In this example, a signal can indicate a presence of one analyte of the 6 different analytes. Such a test can provide a fast and low-cost way to screen a sample for a plurality of analytes (e.g., viral species, bacterial DNA, etc.). Such a multiplexed test can be used for at least about 2, 3, 4, 5, 6, 7, 8, 9, 10, 50, 100, 500, 1,000, or more analytes. Such a multiplexed test can be used for at most about 1,000, 500, 100, 50, 10, 9, 8, 7, 6, 5, 4, 3, or less analytes.

### FURTHER ASPECTS OF THE DISCLSOURE

1. A device for assaying a presence or an absence of an analyte, comprising:
a substrate comprising at least two composite electrodes configured to capture said analyte and detect a signal indicative of said presence or absence of said analyte, upon or subsequent to contact of said analyte with said substrate.
2. A device, comprising:
a three-dimensional membrane-based substrate comprising at least a first location and a second location,
wherein said first location comprises a first substance specific for a first analyte and configured to facilitate generation of a first signal indicative of a presence of said first analyte, upon or subsequent to contact of said first substance with said first analyte, and
wherein said second location comprises a second substance specific for a second analyte different from said first analyte and configured to facilitate generation of a second signal indicative of a presence of said second analyte, upon or subsequent to contact of said second substance with said second analyte.
3. The device of aspect 2, further comprising a detection unit in sensory communication with said first location or said second location.
4. The device of aspect 3, wherein said detection unit is configured to detect a presence or absence of said first signal or said second signal from said first location or said second location, thereby determining a presence or absence of said first analyte or said second analyte.
5. The device of aspect 4, wherein said detection unit is a personal device of a subject.
6. The device of aspect 5, wherein said subject is a person suspected of having a condition or disease, and wherein said presence or absence of said first analyte or said second analyte is indicative of said subject having said condition or disease.
7. The device of aspect 5, wherein said subject is a health care provider.
8. The device of aspect 5, wherein said detection unit is a smartphone.
9. The device of any one of aspects 2-8, wherein said substrate comprises a plurality of locations each comprising a substance specific for a different analyte.
10. The device of any one of aspects 2-9, wherein said substrate comprises a structure.
11. The device of aspect 10, wherein said structure is configured to receive and retain a sample suspected of having said first analyte or said second analyte.
12. The device of aspect 10, wherein said structure comprises said first location and said second location.
13. The device of aspect 10, wherein said substrate comprises a plurality of structures.
14. The device of aspect 13, wherein said plurality of said structures comprises at least a first structure corresponding to said first location and a second structure corresponding to said second location.
15. The device of aspect 13, wherein said first location and said second location are spatially separated.
16. The device of aspect 13, wherein said first location and said second location overlap.
17. The device of aspect 10, wherein said structure is a microstructure.
18. The device of aspect 10, wherein said structure is a well.
19. The device of aspect 10, wherein said structure is a fluidic channel.
20. The device of aspect 19, wherein said fluidic channel is configured to facilitate detection of a different analyte.
21. The device of aspect 10, wherein said structure comprise a plurality of locations each comprising a substance specific for a different analyte.
22. The device of aspect 10, wherein individual locations of said plurality of locations are individually addressable and individually controllable.
23. The device of aspect 10, wherein individual locations of said plurality of locations are addressed and controlled asynchronously.
24. The device of aspect 10, wherein said plurality of locations comprises at least about 10 locations.
25. The device of any one of aspects 2-24, wherein said first analyte or said second analyte comprises nucleic acid molecules.
26. The device of aspect 25, wherein said first substance or said second substance comprises a primer.
27. The device of any one of aspects 2-26, wherein said membrane-based substrate comprises one or more additional locations.
28. The device of aspect 27, wherein said one or more additional locations are configured to act as a positive or negative control.
29. The device of any one of aspects 2-28, further comprising one or more additional membrane-based substrates.
30. The device of any one of aspects 2-29, wherein said device is a portable device.
31. The device of any one of aspects 2-30, wherein said device further comprises one or more bottles configured to contain one or more reagents.
32. The device of aspect 31, wherein said one or more bottles comprise a feedback module.
33. The device of aspect 32, wherein said feedback module is configured to provide information regarding the application of said one or more reagents from said one or more bottles.
34. The device of aspect 32, wherein said feedback module comprises an electronic feedback module.
35. The device of aspect 34, wherein said electronic feedback module comprises a conductive module, a capacitive module, a resistive module, or any combination thereof
36. A method, comprising:
(a) directing a sample suspected of having a first analyte or a second analyte different from said first analyte to a device, said device comprising:
a three-dimensional membrane-based substrate, wherein said membrane-based substrate comprises at least a first location and a second location,
wherein said first location comprises a first substance specific for said first analyte and configured to facilitate generation of a first signal indicative of a presence of said first analyte, upon or subsequent to contact of said first substance with said first analyte, and
wherein said second location comprises a second substance specific for said second analyte and configured to facilitate generation of a second signal indicative of a presence of said second analyte, upon or subsequent to contact of said second substance with said second analyte;
(b) detecting a presence or absence of said first signal or said second signal from said first location or said second location, upon or subsequent to said sample being directed to said device; and
(c) determining a presence or absence of said first analyte or said second analyte, based on said presence or absence of said first signal or said second signal detected in (b).
37. The method of aspect 36, wherein said first signal or said second signal comprises a signal increase relative to a baseline.
38. The method of any one of aspects 36-37, wherein said first signal or said second signal comprises a signal decrease relative to a baseline.
39. The method of any one of aspects 36-38, wherein said sample is from a subject suspected of having a disease or condition.
40. The method of any one of aspects 36-39, wherein said sample is from a subject suspected of being infected with a pathogen.
41. The method of aspect 40, wherein said pathogen is severe acute respiratory syndrome coronavirus-19 (SARS-CoV-2)
42. The method of aspect 40, wherein said first analyte or said second analyte comprises nucleic acid molecules
43. The method of aspect 42, wherein said first substance or said second substance comprises a primer
44. The method of aspect 42, wherein said nucleic acid molecules comprise a ribonucleic acid (RNA) of SARS-CoV-2 or a fragment thereof
45. The method of any one of aspects 36-44, wherein said first signal or said second signal is a colorimetric signal.
46. The method of aspect 45, wherein said detecting said first signal or said second signal comprises detecting a color calibration panel disposed adjacent to said first location or said second location.
47. The method of any one of aspects 36-46, wherein said first signal or said second signal is an electrical signal.
48. The method of aspect 47, wherein said electrical signal is related to a change in a pH of a solution comprising the first or second analyte.
49. The method of any one of aspects 36-48, wherein one of said first signal or said second signal is a colorimetric signal and the other of said first signal or said second signal is an electrical signal.
50. The method of any one of aspects 36-49, wherein said sample has a volume of at least about 500 microliters.
51. The method of any one of aspects 36-50, wherein said sample does not comprise a pH buffer.
52. The method of aspect 51, wherein said sample is mixed with a reaction mixture comprising a pH buffer.
53. The method of any one of aspects 36-52, wherein said sample comprises saliva, blood, or a combination thereof.
54. The method of aspect 53, wherein said sample is taken from a subject using a swab.
55. The method of aspect 54, wherein said swab comprises a breakable head.
56. The method of any one of aspects 36-55, further comprising outputting a report that identifies said presence or absence of said first analyte or said second analyte.
57. The method of aspect 56, wherein said report comprises one or more color codes indicative of said first analyte or said second analyte.
58. The method of aspect 56, further comprising displaying said report on a personal device of a subject.
59. The method of aspect 58, wherein said subject is a subject from which said sample is obtained.
60. The method of aspect 58, wherein said subject is a health care provider.
61. The method of aspect 58, wherein said personal device is a mobile device.
62. The method of aspect 61, wherein said mobile device comprises a light, and wherein said light is configured to illuminate a colorimetric signal.
63. The method of aspect 58, wherein said personal device is in communication with said device.
64. The method of any one of aspects 36-63, wherein said detecting said presence or absence of said first signal or said second signal occurs at a temperature of at least about 35° C.
65. The method of any one of aspects 36-64, wherein said first analyte or said second analyte comprises nucleic acid molecules.
66. The method of any one of aspects 36-65, wherein said first substance or said second substance comprises a primer.
67. A device, comprising:
two or more fluidic chambers;
a fluidic channel between and in fluidic communication with said two or more fluidic chambers; and
a valve disposed adjacent to or within said fluidic channel, said valve (i) comprising a chamber that is compressible or expandable, and (ii) configured to regulate fluid flow between said two or more fluidic chambers upon an actuation of said chamber.
68. The device of aspect 67, wherein said chamber is a plastic encased bubble.
69. The device of aspect 68, wherein said plastic encased bubble is a plastic encased air bubble.
70. The device of any one of aspects 67-69, wherein said actuation comprises an application of a pressure to said chamber.
71. The device of aspect 70, wherein said pressure is a positive pressure.
72. The device of aspect 70, wherein said pressure is applied manually.
73. The device of any one of aspects 67-72, wherein said valve comprises a pressure breakable seal.
74. The device of any one of aspects 67-73, wherein a thickness of a membrane wall of said valve is less than a thickness of a wall of said fluidic channel.
75. The device of aspect 74, wherein a thickness of said membrane wall is at most about 1 millimeter.
76. The device of any one of aspects 67-75, wherein said device is a single tube.
77. The device of any one of aspects 67-76, wherein said device is a cartridge.
78. The device of aspect 77, wherein said cartridge comprises a plurality of valves connecting a plurality of fluidic chambers.
79. The device of any one of aspects 67-78, wherein said device comprises said valve between a sample chamber and a reagent chamber.
80. The device of aspect 79, wherein said sample chamber and said reagent chamber are affixed to a rigid support.
81. The device of any one of aspects 67-80, wherein said chamber is filled with pressurized gas.
82. The device of any one of aspects 67-81, wherein said chamber is filled with non-pressurized gas.
83. The device of any one of aspects 67-82, wherein said device is a portable device.
84. The device of any one of aspects 67-83, wherein said chamber is configured to not break upon said application of said pressure on only one side of said chamber.
85. The device of any one of aspects 67-84, wherein said chamber is inflated.
86. A method, comprising:
(a) directing a fluid to a device comprising:
two or more fluidic chambers;
a fluidic channel between and in fluidic communication with said two or more fluidic chambers; and
a valve disposed adjacent to or within said fluidic channel, said valve (i) comprising a chamber that is compressible or expandable, and (ii) configured to regulate fluid flow between said two or more fluidic chambers upon actuation of said chamber; and
(b) actuating said chamber to regulate fluid flow between said two or more fluidic chambers.
87. The method of aspect 86, wherein said regulating fluid flow comprises bursting said chamber.
88. The method of any one of aspects 86-87, wherein said actuation comprises applying a pressure to said chamber.
89. The method of aspect 88, wherein said pressure is applied on a center of said chamber.
90. The method of aspect 88, wherein said pressure is a pressure of at most about 0.5 megapascals.
91. The method of any one of aspects 86-90, further comprising actuating said chamber a second time.
92. The method of aspect 91, wherein said actuating said chamber said second time comprises applying a pressure to fully break said chamber.
93. A device, comprising:
an inlet configured to receive a sample;
a fluidic channel in fluidic connection with said inlet and a fluidic region downstream of said inlet, said fluidic channel configured to passively or actively flow said sample from said inlet to said fluidic region upon receipt of said sample; and
at least one electrode adjacent to and operably coupled to said fluidic region, said at least one electrode configured to (1) enrich for one or more analytes from said sample in said fluidic region, (2) subject said one or more analytes to one or more reactions under conditions sufficient to yield a signal indicative of a presence or absence of an analyte among said one or more analytes, and (3) detect said signal from said fluidic region, thereby determining said presence or absence of said analyte in said sample.
94. The device of aspect 93, wherein said at least one electrode is configured to apply an electric field of at most about 3 V to concentrate said one or more analytes.
95. The device of any one of aspects 93-94, wherein said at least one electrode is configured to subject said one or more analytes to a temperature from about 30° C. to about 75° C.
96. The device of aspect 95, wherein said temperature is from about 35° C. to about 40° C.
97. The device of any one of aspects 93-96, wherein said device comprises a heating element configured to subject said one or more analytes to a temperature from about 30° C. to about 75° C., and optionally wherein said temperature is from about 35° C. to about 40° C.
98. The device of any one of aspects 93-97, wherein said at least one electrode is functionalized with one or more of nucleotides, oligonucleotides, antimers, antibodies, chelators, or proteins configured to express binding affinity to said one or more analytes.
99. The device of any one of aspects 93-98, wherein said at least one electrode comprises two or more electrodes configured in a concentric arrangement.
100. The device of aspect 99, wherein said two or more concentric electrodes are configured to be sequentially charged to sequentially enrich for said one or more analytes.
101. The device of any one of aspects 93-100, wherein said at least one electrode comprises a material selected from the group consisting of gold, silver, copper, and conductive carbon.
102. The device of aspect 101, wherein said at least one electrode comprises a conductive carbon membrane.
103. The device of any one of aspects 93-102, wherein said device is a portable device.
104. The device of any one of aspects 93-103, wherein said device further comprises one or more bottles configured to contain one or more reagents.
105. The device of aspect 104, wherein said one or more bottles comprise a feedback module.
106. The device of aspect 105, wherein said feedback module is configured to provide information regarding the application of said one or more reagents from said one or more bottles.
107. The device of aspect 105, wherein said feedback module comprises an electronic feedback module, and optionally wherein said electronic feedback module comprises a conductive module, a capacitive module, a resistive module, or any combination thereof
108. The device of any one of aspects 93-107, wherein said at least one electrode comprises a functionalization with one or more of silicon dioxide, titanium oxide, zinc oxide, or nanoparticles thereof.
109. A method, comprising:
(a) directing a sample to a device comprising:
an inlet configured to receive said sample;
a fluidic channel in fluidic connection with said inlet and a fluidic region downstream of said inlet, said fluidic channel configured to passively or actively flow said sample from said inlet to said fluidic region upon receipt of said sample; and
at least one electrode adjacent to and operably coupled to said fluidic region, said at least one electrode configured to (1) enrich for one or more analytes from said sample in said fluidic region, (2) subject said one or more analytes to one or more reactions under conditions sufficient to yield a signal indicative of a presence or absence of a analyte among said one or more analytes, and (3) detect said signal from said fluidic region, thereby determining said presence or absence of said analyte in said sample;
(b) passively or actively flow said sample from said inlet to said fluidic region via said fluidic channel;
(c) using said at least one electrode to, in said fluidic region, enrich for one or more analytes from said sample, and subject said one or more analytes to one or more reactions under conditions sufficient to yield a signal indicative of a presence or absence of an analyte among said one or more analytes; and
(d) detecting said signal from said fluidic region using said at least one electrode, thereby determining said presence or absence of said analyte in said sample.
110. The method of aspect 109, wherein said enriching for one or more analytes comprises applying an electric field to said sample, or wherein said subjecting said one or more analytes to said reaction comprises using said at least one electrode to heat said one or more analytes.
111. The method of any one of aspects 109-110, wherein said at least one electrode comprises a plurality of electrodes, and wherein individual electrodes of said plurality of electrodes are configured to, individually or collectively, perform one or more of said (1)-(3).
112. The method of aspect 111, wherein said one or more analytes comprises one or more nucleic acids, and wherein said heating is sufficient to perform an isothermal amplification reaction of said one or more nucleic acids.
113. The method of any one of aspects 109-112, wherein said detecting said signal comprises detecting an electrical signal.
114. The method of aspect 113, wherein said electrical signal is generated by a change in a pH of said sample.
115. The method of aspect 113, wherein said electrical signal is generated by a change in an electrical property across an electrode due to an oxidation or reduction reaction.
116. The method of aspect 114, wherein said change in said pH is due to an amplification of one or more nucleic acids within said sample.
117. The method of any one of aspects 109-116, wherein said detecting said signal comprises detecting an optical signal.
118. The method of aspect 117, wherein said optical signal is a colorimetric optical signal generated by a change in a pH of said sample.
119. The method of aspect 118, wherein said colorimetric optical signal comprises a change in color of a pH indicator.
120. The method of aspect 117, wherein said device further comprises a color standard positioned to be viewable when performing said detecting.
121. The method of aspect 120, wherein said color standard is used to calibrate for a color of said optical signal.
122. The method of aspect 117, wherein said optical signal is a colorimetric optical signal generated by an enzymatic oxidation or reduction of a substrate.
123. The method of aspect 122, wherein said enzymatic oxidation or reduction of the substrate comprises the use of a horseradish peroxidase.
124. The method of any one of aspects 109-123, further comprising pulsing an electrical current through said at least one electrode to mix said one or more analytes with one or more reagents.
125. A device, comprising:
a membrane-based substrate comprising (1) a recess configured to receive and retain a sample having a volume of less than or equal to about 5 microliters (μL), and (2) a surface comprising a substance specific for an analyte and configured to facilitate generation of a signal indicative of a presence or absence of said analyte in said sample, upon or subsequent to contact of said sample with said surface.
126. The device of aspect 125, wherein said volume is less than or equal to about 1 μL.
127. The device of any one of aspects 125-126, wherein said signal is an electrical signal.
128. The device of aspect 127, wherein said electrical signal is related to a change in a pH of said sample.
129. The device of aspect 127, wherein said electrical signal is generated by a change in an electrical property across an electrode due to an oxidation or reduction reaction.
130. The device of any one of aspects 125-129, wherein said signal is an optical signal.
131. The device of aspect 130, wherein said optical signal is a colorimetric signal.
132. The device of aspect 131, wherein said colorimetric signal is generated by a change in color of a pH indicator.
133. The device of aspect 131, wherein said colorimetric signal is generated by an enzymatic oxidation or reduction of a substrate.
134. The device of aspect 133, wherein said enzymatic oxidation or reduction of a substrate comprises the use of a horseradish peroxidase
135. The device of any one of aspects 125-134, wherein said membrane-based substrate comprises a plurality of recesses.
136. The device of aspect 135, wherein each of said plurality of recesses comprises a surface each comprising a substance specific for a different analyte and configured to facilitate generation of a signal indicative of a presence or absence of said different analytes in said sample, upon or subsequent to contact of said sample with said plurality of recesses.
137. The device of any one of aspects 125-136, further comprising at least one electrode.
138. The device of aspect 137, wherein said at least one electrode is configured to detect said signal.
139. The device of any one of aspects 125-138, further comprising a plurality of membrane-based substrates in an array.
140. The device of aspect 139, wherein each membrane-based substrate of said membrane-based substrates comprises a surface comprising substances specific for a different analyte of a plurality of analytes.
141. The device of any one of aspects 125-140, wherein at least a portion of said membrane-based substrate is adjacent to or part of a conductive region.
142. A method, comprising:
(a) directing a sample to a device, said device comprising a membrane-based substrate having (1) a recess configured to receive and retain less than or equal to about 5 microliters (μL) said sample and (2) a surface comprising a substance specific for a analyte and configured to facilitate generation of a signal indicative of a presence or absence of said analyte in said sample, upon or subsequent to contact of said sample with said surface; and
(b) detecting said signal from said surface upon or subsequent to contact of said sample with said surface, thereby determining a presence or absence of said analyte in said sample.
143. The method of aspect 142, wherein said sample is from a subject suspected of having a disease or condition
144. The method of any one of aspects 142-143, wherein said sample is from a subject suspected of being infected with a pathogen
145. The method of aspect 144, wherein said pathogen is severe acute respiratory syndrome coronavirus-19 (SARS-CoV-2)
146. The method of aspect 144, wherein said analyte comprises nucleic acid molecules
147. The method of aspect 146, wherein said substance comprises a primer
148. The method of aspect 146, wherein said nucleic acid molecules comprise a ribonucleic acid (RNA) of SARS-CoV-2 or a fragment thereof
149. The method of any one of aspects 142-148, wherein said signal is an electrical signal.
150. The method of aspect 149, wherein said electrical signal is related to a change in a pH of said sample.
151. The method of aspect 149, wherein said electrical signal is generated by a change in an electrical property across an electrode due to an oxidation or reduction reaction.
152. The method of any one of aspects 142-151, wherein said signal is an optical signal.
153. The method of aspect 152, wherein said optical signal is a colorimetric signal.
154. The method of aspect 153, wherein said colorimetric signal is generated by a change in color of a pH indicator.
155. A device, comprising:
a membrane-based substrate comprising a recess configured to receive and retain (i) a sample having a volume of less than or equal to about 2 milliliters and (ii) a substance specific for a analyte and configured to facilitate generation of a signal indicative of a presence or absence of said analyte in said sample, upon or subsequent to contact of said sample with said surface; and a control unit configured to subject said sample and said substance to one or more reactions under conditions sufficient to generate said signal within 60 minutes (min) subsequent to receipt of said sample.
156. The device of aspect 155, wherein said volume is less than or equal to about 1 milliliter.
157. The device of any one of aspects 155-156, wherein said signal is an electrical signal.
158. The device of aspect 157, wherein said electrical signal is related to a change in a pH of said sample.
159. The device of aspect 157, wherein said electrical signal is generated by a change in an electrical property across an electrode due to an oxidation or reduction reaction.
160. The device of any one of aspects 155-159, wherein said signal is an optical signal.
161. The device of aspect 160, wherein said optical signal is a colorimetric signal.
162. The device of aspect 161, wherein said colorimetric signal is generated by a change in color of a pH indicator.
163. The device of aspect 161, wherein said colorimetric signal is generated by an enzymatic oxidation or reduction of a substrate.
164. The device of aspect 163, wherein said enzymatic oxidation or reduction of a substrate comprises the use of a horseradish peroxidase
165. The device of any one of aspects 155-164, wherein said membrane-based substrate comprises a plurality of recesses.
166. The device of aspect 165, wherein each recess of said plurality of recesses comprises a surface each comprising a substance specific for a different analyte and configured to facilitate generation of a signal indicative of a presence or absence of said different analytes in said sample, upon or subsequent to contact of said sample with said plurality of recesses.
167. A method, comprising:
(a) directing (i) a sample having a volume of less than or equal to about 2 milliliters and (ii) a substance to a device, said device comprising a recess configured to receive and retain said sample and said substance, which substance is specific for an analyte and configured to facilitate generation of a signal indicative of a presence or absence of said analyte in said sample, upon or subsequent to contact of said sample with said surface;
(b) subjecting said sample and said substance to one or more reactions under conditions sufficient to generate said signal; and
(c) detecting said signal from said substrate, thereby determining a presence or absence of said analyte in said sample, wherein (a)-(c) are separated in time by less than or equal to about 45 minutes (min).
168. The method of aspect 167, wherein (a)-(c) are separated in time by less than or equal to 15 min.
169. The method of any one of aspects 167-168, wherein (a)-(c) are separated in time by less than or equal to 10 min.
170. The method of any one of aspects 167-169, wherein said one or more reactions are one or more amplification reactions.
171. The method of aspect 170, wherein said one or more amplification reactions are an isothermal nucleic acid amplification reaction.
172. The method of any one of aspects 167-171, wherein the device further comprises a control unit.
173. The method of aspect 172, wherein said control unit is configured to subject said sample and said substance to said one or more reactions under conditions sufficient to generate said signal subsequent to receipt of said sample
174. The method of aspect 172, wherein said control unit is an electronic unit.
175. The method of aspect 174, wherein said control unit comprises at least one electrode.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations, or equivalents. It is intended that the following claims define the scope of the invention and that methods and structures within the scope of these claims and their equivalents be covered thereby.

1.-77. (canceled)

78. A device, comprising:
an inlet configured to receive a sample comprising one or more analytes;
a fluidic channel in fluidic connection with said inlet and a fluidic region downstream of said inlet, said fluidic channel configured to passively or actively flow said sample from said inlet to said fluidic region upon receipt of said sample, wherein said fluidic region comprises at least one composite electrode adjacent to and operably coupled to said fluidic region, wherein said at least one composite electrode is configured to enrich for said one or more analytes from said sample in said fluidic region.

79. The device of claim 78, wherein said inlet is an access port or a sample bottle.

80. The device of claim 78, wherein said device further comprises one or more reservoirs configured to contain one or more reagents.

81. The device of any one of claim 78, wherein said composite electrode comprises paper, cellulose, nitro-cellulose, nylon, polytetrafluoroethylene, another polymer, or any combination thereof, wherein said composite electrode comprises at least a first location and a second location, and wherein said one or more analytes comprising a first analyte and a second analyte.

82. The device of claim 81, wherein said first location comprises a first substance specific for said first analyte and configured to facilitate generation of a first signal indicative of a presence of said first analyte, upon or subsequent to contact of said first substance with said first analyte, and wherein said second location comprises a second substance specific for said second analyte different from said first analyte and configured to facilitate generation of a second signal indicative of a presence of said second analyte, upon or subsequent to contact of said second substance with said second analyte.

83. The device of claim 81, wherein said first location and said second location are spatially separated.

84. The device of any one of claim 81 wherein said first analyte or said second analyte comprises nucleic acid molecules, and wherein said first substance or said second substance comprises a primer.

85. The device of any one of claim 81, wherein said composite electrode comprises one or more additional locations configured to act as a positive or negative control.

86. The device of any one of claim 78, wherein said composite electrode is a nylon-based membrane or a cellulose-based membrane.

87. The device of any one of claim 78, wherein said at least one composite electrode comprises an conductive element fully or partially in, on, or adjacent to an absorptive element.

88. The device of claim 87, wherein the conductive element is conductive carbon.

89. The device of any one of claim 87, wherein said composite electrode or said absorptive element comprises a functionalization with one or more of silicon dioxide, titanium oxide, zinc oxide, or nanoparticles thereof.

90. A method, comprising:
(a) directing a sample comprising one or more analytes to a device, said device comprising:
an inlet configured to receive said sample;
a fluidic channel in fluidic connection with said inlet and a fluidic region downstream of said inlet, said fluidic channel configured to passively or actively flow said sample from said inlet to said fluidic region upon receipt of said sample, wherein said fluidic region comprises at least one composite electrode adjacent to and operably coupled to said fluidic region,
wherein said at least one composite electrode is configured to enrich for said one or more analytes from said sample in said fluidic region.
(b) passively or actively flowing said sample from said inlet to said fluidic region via said fluidic channel; and
(c) enriching for said one or more analytes from said sample in said fluidic region.

91. The method of claim 90, wherein said enriching for said one or more analytes comprises applying an electric field to said sample.

92. The method of claim 90, further comprising: subjecting said one or more analytes to one or more reactions under conditions sufficient to yield a signal indicative of a presence or absence of an analyte among said one or more analytes.

93. The method of claim 92, further comprising: detecting said signal from said fluidic region, thereby determining said presence or absence of said analyte in said sample.

94. The method of any one of claim 90, wherein said one or more analytes comprises one or more nucleic acids, and wherein said one or more reactions comprises an isothermal amplification reaction of said one or more nucleic acids.

95. The method of any one of claim 91, wherein said applying said electric field comprises applying at most about 50 V to concentrate said one or more analytes.

96. The method of any one of claim 90, further comprising: sequentially charging said at least one composite electrode to sequentially enrich for said one or more analytes, wherein said at least one composite electrode comprises two or more composite electrodes.

97. The method of any one of claim 93, wherein said detecting comprises detecting an optical signal.
